(19) United States
(12) Patent Application Publication
(10) Pub. No.: US 2009/0162351 A1
(43) Pub. Date: Jun. 25, 2009

(54) Transdiscal administration of inhibitors of p38 MAP kinase

(75) Inventors: Laura J. Brown, Hamilton Square, NJ (US); Jeffery C. Geesin, Doylestown, PA (US); Carrie H. Fang, Pittstown, NJ (US); John J. Siekierka, Towaco, NJ (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc., Raynham, MA (US)

(21) Appl. No.: 12/005,060

(22) Filed: Dec. 21, 2007

(51) Int. Cl. A61K 39/395; A61K 31/496; A61K 31/4439; A61P 19/00; A61K 35/12; A61K 31/519

(52) U.S. Cl. 424/133.1; 514/254.09; 514/341; 514/253.09; 514/259.1; 424/93.7

(57) ABSTRACT

The present invention relates to methods, formulations and kits for administering a p38 MAP kinase inhibitor or other therapeutic agent into an intervertebral disc, such as a diseased disc, for example, for purposes of prevention and treatment of degenerative and other disorders.

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus with its cushioning qualities. These nucleus pulposus cells may also secrete small amounts of cytokines as well as matrix metalloproteinases (“MMPs”). These cytokines and MMPs help regulate the metabolism of the nucleus pulposus cells.

In some instances of disc degeneration disease (DDD), gradual degeneration of the intervertebral disc is caused by mechanical instabilities in other portions of the spine. In these instances, increased loads and pressures on the nucleus pulposus cause the cells to emit larger than normal amounts of the above-mentioned cytokines. In other instances of DDD, genetic factors such as programmed cell death or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins.

As DDD progresses, the toxic levels of the cytokines present in the nucleus pulposus begin to degrade the extracellular matrix. In particular, the MMPs (under mediation by the cytokines) begin cleaving the water-retaining portions of the proteoglycans, thereby reducing their water-retaining capabilities. This degradation leads to a less flexible nucleus pulposus, and so changes the load pattern within the disc, thereby possibly causing delamination of the annulus fibrosus. These changes cause more mechanical instability, thereby causing the cells to emit even more cytokines, typically thereby upregulating MMPs. As this destructive cascade continues and DDD further progresses, the disc begins to bulge (“a herniated disc”), and then ultimately ruptures, causing the nucleus pulposus to contact the spinal cord and produce pain.

Accordingly, there is a need for effective prevention and treatment of degenerating discs.

### SUMMARY OF THE INVENTION

The present inventors have developed a number of procedures for efficaciously treating degenerative disc disease by drug therapy.

In accordance with the present invention, the present inventors have developed a method of treating an intervertebral disc in which an inhibitor of a pro-inflammatory cytokine, such as a p38 MAP kinase inhibitor, is administered transdiscally (e.g., the target tissue is a degenerating disc).

Since p38 mitogen-activated protein (MAP) kinase plays a central role in inflammation, transdiscal injection of p38 MAP kinase inhibitors blocks the production of pro-inflammatory cytokines, such as, for example, TNF-α, IL-1, IL-6 and IL-8, and blocks nitric oxide (NO) production, prostaglandin E₂ (PGE₂) production and proteoglycan degradation. It is believed that the p38 MAP kinase site regulates the production of TNF-α, IL-1 and COX-2 enzyme.

There are several advantages to directly administering these therapeutic inhibitors to a targeted disc:

First, since it is known that many cytokines (such as p38 MAP kinase, interleukins and TNF-α) also play roles in mediating the degradation of the extracellular matrix (ECM) of the nucleus pulposus, injecting an antagonist or inhibitor of these proteins directly into the disc prevents the target cytokine from inducing any further ECM degradation. In effect, the transdiscal administration of a cytokine antagonist arrests the aging process of the degenerating disc. Accordingly, the present invention seeks to treat the degenerative disc at a much earlier stage of DDD, thereby preventing degradation of the ECM.

Second, it is further known that nerve ending nociceptors are present within the annulus fibrosus, and that cytokines such as TNF irritate nerves. It is believed that injecting an antagonist of TNF, including a p38 MAP kinase inhibitor, into the disc space also prevents the TNF from causing nerve irritation within the disc. Thus, the pain attributed to irritation of these nerves can be efficiently eliminated.

Third, since the annulus fibrosus portion of the disc comprises a relatively dense fibrosus structure, this outer component of the disc may provide a suitable depot for the cytokine antagonist, thereby increasing its half-life in the disc.

Fourth, since it is believed that many of the problematic cytokines are actually secreted by either nucleus pulposus or annulus fibrosus cells, transdiscal injection of the antagonists will advantageously attack the problematic cytokines at their source of origination.

Fifth, when the antagonist is a high specificity cytokine antagonist, it inhibits the cytokine of interest, and the cytokine antagonist may be combined with other therapeutic agents (such as TGF-β or mesenchymal stem cells) that can also be injected into the disc without reducing the effectiveness of those agents.

The disintegration of articular cartilage induces production of pro-inflammatory cytokines (interleukin 1α-IL-1α, interleukin 1β-IL-1β, tumor necrosis factor α-TNF-α, interleukin 6-L-6, leukaemic inhibitor factor-LIF, interleukin 8-IL-8, interleukin 17-IL-17, interleukin 18-IL-18 and others) through the synovial membrane. Cytokines diffuse into the articular cartilage, which produces matrix metalloproteinases (MMPs). MMPs play an important role in the destruction of proteoglycans, collagen and cartilage matrix. The above mentioned cytokines take part in inflammatory and catabolic processes because they disrupt the normal balance of catabolism and new matrix synthesis, inhibit cartilage collagen and aggrecan production, stimulate chondrocytes to produce MMPs and inducible NO-synthase (iNO), induce nitric oxide (NO) production (which induces PGE₂ production), increase the amount of inflammatory cells in the joint, induce and decrease a proliferation of chondrocytes, inhibit proteoglycan synthesis and stimulate their disintegration (which stimulates glycosaminoglycan (GAG) release), induce apoptosis of chondrocytes and decrease their viability. The p38 MAP kinase inhibitors are a class of drugs that target p38 MAP kinase pathway and can inhibit NO and PGE₂ production.

Accordingly, one aspect of the present invention, there is provided a method of treating an intervertebral disc having a nucleus pulposus, comprising transdiscally administering a formulation comprising a cytokine antagonist, such as a p38 MAP kinase inhibitor, into an intervertebral disc.

In one embodiment, the invention includes a method of treating degenerative disc disease in an intervertebral disc having a nucleus pulposus and an annulus fibrosus, comprising transdiscally administering an effective amount of a formulation comprising a p38 MAP kinase inhibitor into an intervertebral disc. In one embodiment, the invention includes preventing degenerating disc disease in an intervertebral disc having a nucleus pulposus and an annulus fibrosus, comprising transdiscally administering an effective amount of a formulation comprising a p38 MAP kinase inhibitor into an intervertebral disc.

The p38 MAP kinase inhibitors can be one or more of the p38 MAP kinase inhibitors disclosed herein. For example, in one embodiment, the p38 MAP kinase inhibitor is selected from the group consisting of:
i) diaryl imidizole;
ii) N,N′-diaryl urea;
iii) N,N-diaryl urea;
iv) benzophenone;
v) pyrazole ketone;
vi) indole amide;
vii) diamides;
viii) quinazoline;
ix) pyrimido [4,5-d]pyrimidinone;
x) pyridylamino-quinazolines;
xi) JNJ 3026582 (RWJ 67657);
xii) JNJ 17089540 (RWJ 669307);
xiii) JNJ 7583979 (RWJ 351958);
xiv) SCIO-282 (SD 282); and
xv) SCIO-469 (SD 469).

In one embodiment, the p38 MAP kinase inhibitor is selected from the group consisting of:
a) JNJ 3026582 (RWJ 67657),
b) JNJ 17089540 (RWJ 669307),
c) JNJ 7583979 (RWJ 351958),
d) SCIO-282 (SD 282), and
e) SCIO-469 (SD 469).

In one embodiment, the p38 MAP kinase inhibitor has at least 3 cyclic groups. In one embodiment, the p38 MAP kinase inhibitor is substantially water insoluble. In another embodiment, the p38 MAP kinase inhibitor is water soluble.

In one embodiment, the p38 MAP kinase inhibitor is an aryl-pyridinyl heterocyle, e.g., a 1-aryl-2-pyridinyl heterocycle. For example, the 1-aryl-2-pyridinyl heterocycle can be selected from the group consisting of:
a) 4,5 substituted imidazole;
b) 1,4,5 substituted imidizole;
c) 2,4,5 substituted imidizole;
d) 1,2,4,5 substituted imidizole; and
e) non-imidizole 5-membered ring heterocycle.

In one embodiment, the formulation is administered in an amount of less than about 1 cc. In one embodiment, the p38 MAP kinase inhibitor is administered in a dose of less than about 1 cc. In one embodiment, the formulation is administered in a volume of between about 0.03 ml and about 0.3 ml. In one embodiment, the p38 MAP kinase inhibitor is present in the formulation in an amount of no more than about 0.5 mg. In one embodiment, the p38 MAP kinase inhibitor is administered in a dosage to produce a local tissue concentration of between about 1 to about 50 μM. In one embodiment, the p38 MAP kinase inhibitor is present in the formulation in an amount of at least about 100 mg/ml.

In one embodiment, the formulation is administered in an amount effective to reduce pain. In one embodiment, the formulation is administered in an amount effective to inhibit degradation of an extracellular matrix of the nucleus pulposus.

In one embodiment, the administration comprises providing the formulation in a patch attached to an outer wall of the annulus fibrosus. In one embodiment, the administration comprises providing the formulation in a depot at a location closely adjacent to an outer wall of the annulus fibrosus. In one embodiment, the administration comprises providing the formulation in a depot at a location closely adjacent to an endplate of an adjacent vertebral body. In one embodiment, the formulation is provided closely adjacent the outer wall of the annulus fibrosus. In one embodiment, the formulation is injected into the nucleus pulposus. In one embodiment, the formulation is injected into the annulus fibrosus.

In one embodiment, a portion of the nucleus pulposus is removed prior to transdiscally administering the formulation.

In one embodiment, the administration is performed through a needle.

In one embodiment, the formulation is administered through a drug pump.

In one embodiment, the degenerating disc is an intact disc. In one embodiment, the degenerating disc is a ruptured disc. In one embodiment, the degenerating disc is delaminated. In one embodiment, the degenerating disc has fissures.

In one embodiment, more than one p38 MAP kinase inhibitor is administered. For example, the formulation can contain more than one p38 MAP kinase inhibitor.

In one embodiment, an additional therapeutic agent is administered. In one embodiment, the administered formulation further comprises at least one additional therapeutic agent.

In one embodiment, the additional therapeutic agent can include one or more of the following agents:
i) a growth factor,
ii) viable cells,
iii) an MMP antagonist,
iv) a monoclonal anti-TNFα antibody,
v) rapamycin,
vi) a COX-2 antagonist,
vii) an antagonist of NO synthesis,
viii) an anti-oxidant,
ix) an anti-proliferative agent,
x) an anti-apoptotic agent,
xi) a non-steroidal anti-inflammatory agent,
xii) glycosaminoglycans,
xiii) microparticles,
xiv) a caspase inhibitor,
xv) an inhibitor of pro-inflammatory interleukin,
xvi) an inhibitor of PLA₂ enzyme,
xvii) tetracycline analogs, and
xviii) IGF-I and II.

In one embodiment, the additional therapeutic agent is a growth factor. In one embodiment, the formulation comprises a growth factor present in an amount effective to repair disc tissue. In one embodiment, the growth factor is provided by platelet concentrate.

In one embodiment, the additional therapeutic agent is viable cells, such as postpartum cells and mesenchymal stem cells. In one embodiment, the cells are autologous. In one embodiment, the cells are provided in a concentrated form.

In one embodiment, the additional therapeutic agent is plasmid DNA.

In one embodiment, the additional therapeutic agent is postpartum-derived cells (PPDCs), which are also known as postpartum cells. The PPDCs can be placenta-derived cells (PDCs) or human Umbilical Tissue-derived Cells (hUTCs). Methods for isolating and collecting PPDCs are described in U.S. application Ser. No. 10/877,446 and 10/877,012, which are incorporated by reference herein in their entirety.

In one embodiment, the invention includes a method of treating or preventing degenerative disc disease in an intervertebral disc having a nucleus pulposus, comprising transdiscally administering an effective amount of a formulation comprising an antagonist of COX-2 enzyme into an intervertebral disc. In another embodiment, the additional therapeutic agent is an antagonist of NO synthase. In one embodiment, the antagonist of NO synthase is selected from the group consisting of N-iminoethyl-L-lysine (L-NIL), and N^{G}-monomethyl-L-arginine.

In one embodiment, the additional therapeutic agent is selected from the group consisting of: anti-proliferative agents, anti-inflammatory agents, and antibodies.

In one embodiment, the additional therapeutic agent is an anti-proliferative agent. In one embodiment, the anti-proliferative agent is a CDK inhibitor. In one embodiment, the CDK inhibitor is provided in an about 0.1 to about 10 μM dose. In one embodiment, the anti-proliferative agent is rapamycin. In one embodiment, the rapamycin is provided in an about 0.1 to about 10 μM dose. In one embodiment, the anti-proliferative agent is selected from the group consisting of rapamycin and JNJ 7706621. JNJ 7706621 (RWJ 387252) is (4-[5-Amino-1-(2,6-difluoro-benzoyl)-1H-[1,2,4]triazol-3-ylamino]-benzenesulfonamide) (developed by Johnson & Johnson) that blocks cell cycle progression through inhibition of cyclin-dependent kinases (CDKs) and Aurora kinases. The molecular weight for JNJ 7706621 is 394. JNJ 7706621 has a solubility of 0.006 mg/ml at pH 2 and solubility of 0.017 mg/ml at pH 7.4. JNJ 7706621 exhibits activity against Aurora (IC₅₀=11 nM) and VEGFR2 (IC50=154 nM). It is very potent towards CDK1 and CDK2 (IC₅₀ 9 and 4 nM, respectively), and inhibits proliferation of many different human cancer cell lines. JNJ 7706621 shows single agent antitumor activity in human tumor xenograft models. JNJ 7706621 inhibits cell proliferation in culture (IC₅₀=337±123 nM in 9 cell lines). JNJ 7706621 induced G2M arrest, endoreduplication and apoptosis. JNJ 7706621 is represented by the structure in FIG. 13B.

In one embodiment, the anti-inflammatory agent is selected from the group consisting of tolmetin, tepoxalin, suprofen, tiaprofenic acid, centella (ETCA), madecassoside, rhein, diacerein, feverfew, batimastat and ORC (Interceed).

In one embodiment, the additional therapeutic agent is an anti-apoptotic agent. In one embodiment, the anti-apoptotic agent is selected from the group consisting of EPO, erythropoetin mimetic peptides, IGF-I, IGF-II, and caspase inhibitors.

In one embodiment, the formulation further comprises a sustained release device. In one embodiment, the sustained release device comprises a hydrogel. In one embodiment, the sustained release device provides controlled release. In one embodiment, the sustained release device provides continuous release. In one embodiment, the sustained release device provides intermittent release. In one embodiment, the sustained release device comprises a biosensor. In one embodiment, the sustained release device comprises a plurality of microspheres. In one embodiment, the sustained delivery device is a polymer. In one embodiment, the sustained release device comprises an inflammatory-responsive delivery system.

In one embodiment, the antagonist is predominantly released from the sustained delivery device by its diffusion through the sustained delivery device. In one embodiment, the antagonist is predominantly released from the sustained delivery device by biodegradation of the sustained delivery device.

In one embodiment, the invention includes a method of preventing or treating a degenerating intervertebral disc, comprising the steps of:
a) determining a level of a pro-inflammatory protein within the disc,
b) comparing the level against a pre-determined level of the pro-inflammatory protein, and
c) injecting a p38 MAP kinase inhibitor into the disc.

In one embodiment, the invention includes a method of preventing or therapeutically treating a degenerating intervertebral disc, comprising the steps of:
a) determining a level of a pro-inflammatory protein within the disc,
b) comparing the level against a pre-determined level of the pro-inflammatory protein, and
c) injecting a p38 MAP kinase inhibitor into the disc.

In one embodiment, the pro-inflammatory protein is an interleukin. For example, the predetermined level for the interleukin is at least 100 pg/ml. In one embodiment, the pro-inflammatory protein is p38 MAP kinase, an interleukin-6. In one embodiment, the pro-inflammatory protein is p38 MAP kinase, an interleukin-1. In one embodiment, the predetermined level for the interleukin-6 is at least 100 pg/ml. For example, the predetermined level for the interleukin-6 is at least 250 pg/ml. In one embodiment, the pro-inflammatory protein is an interleukin-8. For example, the predetermined level for the interleukin-8 is at least 500 pg/ml. In one embodiment, the pro-inflammatory protein is PGE₂. For example, the predetermined level for PGE₂ is at least 1000 pg/ml. In one embodiment, the pro-inflammatory protein is TNF-α. For example, the predetermined level for TNF-α can be at least 20 pg/ml, at least 30 pg/ml or at least 1000 pg/disc.

In one embodiment, the invention includes a method of preventing degeneration of an intervertebral disc in a human individual, comprising:
a) determining a genetic profile of the individual,
b) comparing the profile of the individual against a pre-determined genetic profile level of at-risk humans,
c) determining that the individual is an at-risk patient, and
d) injecting a formulation comprising an effective amount of a p38 MAP kinase inhibitor into a disc of the individual.

In one embodiment, the invention includes a method of treating degenerative disc disease in an intervertebral disc having a nucleus pulposus and an annulus fibrosus, comprising transdiscally administering an effective amount of a formulation comprising p38 MAP kinase inhibitor into an intervertebral disc, wherein said p38 MAP kinase inhibitor is selected from the group consisting of: JNJ 3026582 (RWJ 67657), JNJ 17089540 (RWJ 669307), JNJ 7583979 (RWJ 351958), SCIO-282 (SD 282) and SCIO-469 (SD 469).

In one embodiment, the invention includes a method of inhibiting GAG degradation in an intervertebral disc having a nucleus pulposus and an annulus fibrosus, comprising transdiscally administering an effective amount of a formulation comprising p38 MAP kinase inhibitor into an intervertebral disc, wherein said p38 MAP kinase inhibitor is selected from the group consisting of: JNJ 3026582 (RWJ 67657), JNJ 17089540 (RWJ 669307), JNJ 7583979 (RWJ 351958), SCIO-282 (SD 282) and SCIO-469 (SD 469).

In one embodiment, the invention includes a method of inhibiting NO production in an intervertebral disc having a nucleus pulposus and an annulus fibrosus, comprising transdiscally administering an effective amount of a formulation comprising p38 MAP kinase inhibitor into an intervertebral disc, wherein said p38 MAP kinase inhibitor is selected from the group consisting of: JNJ 3026582 (RWJ 67657), JNJ 17089540 (RWJ 669307), JNJ 7583979 (RWJ 351958), SCIO-282 (SD 282) and SCIO-469 (SD 469).

In one embodiment, the invention includes a method of inhibiting PGE₂ synthesis in an intervertebral disc having a nucleus pulposus and an annulus fibrosus, comprising transdiscally administering an effective amount of a formulation comprising p38 MAP kinase inhibitor into an intervertebral disc, wherein said p38 MAP kinase inhibitor is selected from the group consisting of: JNJ 3026582 (RWJ 67657), JNJ 17089540 (RWJ 669307), JNJ 7583979 (RWJ 351958), SCIO-282 (SD 282) and SCIO-469 (SD 469).

The invention described herein also encompasses formulations comprising at least one p38 MAP kinase inhibitor and at least one an additional therapeutic agent. For example, the additional therapeutic agent or agents can be any of the agents disclosed herein. In some embodiments, the agents can be present in any of the amounts and/or dosages disclosed herein.

In one embodiment, the additional therapeutic agent can be administered prior to, simultaneously with, or subsequent to administration of the p38 MAP kinase inhibitor.

The invention also includes a method of treating degenerative disc disease in an intervertebral disc having a nucleus pulposus and an annulus fibrosus, comprising transdiscally administering an effective amount of a formulation comprising at least one of: an anti-proliferative agent, an anti-inflammatory agent, a cytokine antagonist, an antibody, an IL-6 antibody, or a MMP inhibitor or antagonist, a growth factor, mesenchymal stem cells, a monoclonal anti-TNFα antibody, rapamycin, a COX-2 antagonist, an antagonist of NO synthesis, an anti-oxidant, an anti-apoptotic agent, non-steroidal anti-inflammatory agent, glycosaminoglycans, mesenchymal stem cells, a TNFα antagonist, batimastat, rhein, diacerein, or rhGDF-5.

The invention also encompasses methods of inhibiting proteoglycan degradation in an intervertebral disc having a nucleus pulposus and an annulus fibrosus, comprising transdiscally administering an effective amount of a formulation comprising diacerein or rhein into an intervertebral disc. It also encompasses methods of inhibiting nitric oxide production in an intervertebral disc having a nucleus pulposus and an annulus fibrosus, comprising transdiscally administering an effective amount of a formulation comprising diacerein or rhein into an intervertebral disc.

The invention also encompasses methods of inhibiting glycosaminoglycan release in an intervertebral disc having a nucleus pulposus and an annulus fibrosus, comprising transdiscally administering an effective amount of a formulation comprising an antiproliferative compound (for example, any such compound described herein) into an intervertebral disc. It also encompasses methods of inhibiting nitric oxide production in an intervertebral disc having a nucleus pulposus and an annulus fibrosus, comprising transdiscally administering an effective amount of a formulation comprising an antiproliferative compound into an intervertebral disc.

The invention also encompasses methods of inhibiting TNFα-induced nitric oxide production in an intervertebral disc having a nucleus pulposus and an annulus fibrosus, comprising transdiscally administering an effective amount of a formulation comprising a TNFα antagonist into an intervertebral disc. It also encompasses methods of inhibiting TNFα-induced PGE₂ synthesis in an intervertebral disc having a nucleus pulposus and an annulus fibrosus, comprising transdiscally administering an effective amount of a formulation comprising a TNFα antagonist into an intervertebral disc.

The invention also encompasses methods of inhibiting glycosaminoglycan release in an intervertebral disc having a nucleus pulposus and an annulus fibrosus, comprising transdiscally administering an effective amount of a formulation comprising an IL-6 antibody into an intervertebral disc. It also encompasses methods of inhibiting nitric oxide production in an intervertebral disc having a nucleus pulposus and an annulus fibrosus, comprising transdiscally administering an effective amount of a formulation comprising an IL-6 antibody into an intervertebral disc. It also encompasses methods of inhibiting PGE₂ synthesis in an intervertebral disc having a nucleus pulposus and an annulus fibrosus, comprising transdiscally administering an effective amount of a formulation comprising an IL-6 antibody into an intervertebral disc.

The invention also encompasses methods of inhibiting nitric oxide production in an intervertebral disc having a nucleus pulposus and an annulus fibrosus, comprising transdiscally administering an effective amount of a formulation comprising batimastat into an intervertebral disc.

In one embodiment, batimastat can be administered in a dose of about 1-10 μM.

The invention also encompasses methods of inhibiting TNFα-induced glycosaminoglycan release in an intervertebral disc having a nucleus pulposus and an annulus fibrosus, comprising transdiscally administering an effective amount of a formulation comprising an antiproliferative compound into an intervertebral disc.

In one embodiment, diacerein can be administered in a dose of about 0.054 to 54 μM.

In one embodiment, rhein can be administered in a dose of about 0.035-35 μM.

Also encompassed within the scope of the invention are kits comprising one or more p38 MAP kinase inhibitors and/or formulations disclosed herein.

### BRIEF DESCRIPTION OF THE DRAWINGS

FIG. 1A is a bar graph of the effects of diacerein and rhein on inhibition of GAG degradation in the presence or absence of 10 ng/ml of IL-1β. Y axis represents percent of GAG inhibition and the X axis represents diacerein and rhein in μM. In addition, non-treated (NT) and Batimastat (Bat) treated tissues were tested.

FIG. 1B is a bar graph of the effects of diacerein and rhein on inhibition of cytotoxicity in the presence or absence of 10 ng/ml of IL-1β. Y axis represents LDH level (492 nm) and the X axis represents diacerein and rhein in μM. In addition, non-treated (NT), killed chondrocytes 2×10⁶ cells/ml (KC) and Batimastat (Bat) treated tissues were tested.

FIG. 1C is a bar graph of the effects of diacerein and rhein on inhibition of PGE₂ synthesis in the presence or absence of 10 ng/ml of IL-1β. Y axis represents PGE₂ levels in pg/ml and the X axis represents diacerein and rhein in μM. In addition, non-treated (NT) and Batimastat (Bat) treated tissues were tested.

FIG. 1D is a bar graph of the effects of diacerein and rhein on inhibition of NO production in the presence or absence of 10 ng/ml of IL-1β. Y axis represents NO levels in μM and the X axis represents diacerein and rhein in μM. In addition, non-treated (NT) and Batimastat (Bat) treated tissues were tested.

FIG. 2A is a bar graph of the effects of JNJ 7706621 and rapamycin (Rap) on inhibition of NO production in the presence or absence of 7.5 ng/ml of IL-1β. The Y axis represents NO levels in μM and the X axis represents JNJ 7706621 in μM. In addition, non-treated (NT) and Batimastat (Bat) treated tissues were tested.

FIG. 2B is a bar graph of the effects of JNJ 7706621 and rapamycin (Rap) on inhibition of PGE₂ synthesis in the presence or absence of 7.5 ng/ml of IL-1β. The Y axis represents PGE₂ levels in pg/ml and the X axis represents JNJ 7706621 in μM. In addition, non-treated (NT) and Batimastat (Bat) treated tissues were tested.

FIG. 2C is a bar graph of the effects of JNJ 7706621 and rapamycin (Rap) on inhibition of GAG degradation in the presence or absence of 7.5 ng/ml of IL-1β. The Y axis represents GAG μg and the X axis represents JNJ 7706621 in μM. In addition, non-treated (NT) and Batimastat (Bat) treated tissues were tested.

FIG. 3A is a bar graph of the effect of rapamycin (Rap) on cytotoxicity in the presence or absence of IL-1β. The Y axis represents LDH level (492 nm) and the X axis represents rapamycin in μM. In addition, non-treated (NT), Batimastat (Bat) and killed chondrocytes (KC) treated tissues were tested.

FIG. 3B is a bar graph of the effect of JNJ 7706621 on cytotoxicity in the presence or absence of IL-1β. The Y axis represents LDH level (492 nm) and the X axis represents JNJ 7706621 in μM. In addition, non-treated (NT) and Batimastat (Bat) treated tissues were tested.

FIG. 4 is a bar graph of the effect of REMICADE® on inhibition of GAG degradation. ARC tissues prepared using human chondrocytes and were treated with REMICADE® in the presence or absence of either 5 ng/ml of TNFα or IL-1β at four and five days. For studies testing the efficacy of antibodies, it was necessary to match the cells with the species that each antibody targets. The Y axis represents GAG levels in μg/ml and the X axis represents REMICADE® concentration in ng/ml.

FIGS. 5A-C are bar graphs of the effects of REMICADE® on inhibition of PGE₂ synthesis, NO production and GAG degradation. Human chondrocyte generated ARC tissues were treated with REMICADE® in the presence or absence of 10 ng/ml of TNFα and 5 ng/ml of IL-1β. FIG. 5A is a bar graph of the effects of REMICADE® on inhibition of PGE₂ synthesis. The Y axis represents PGE₂ in pg/ml and the X axis represents REMICADE® concentration in ng/ml. FIG. 5B is a bar graph of the effects of REMICADE® on inhibition of NO production. The Y axis represents NO in μM and the X axis represents REMICADE® concentration in ng/ml. FIG. 5C is a bar graph of the effects of REMICADE® on inhibition of GAG degradation. The Y axis represents GAG in μg/ml and the X axis represents REMICADE® concentration in ng/ml.

FIG. 6A is a bar graph of the effect of a human monoclonal antibody against Il-6 on inhibition of PGE₂ synthesis in human ARCs. The Y axis represents the PGE₂ concentration in pg/ml and the X axis represents the IL-6 monoclonal antibody in the following conditions: presence or absence of 25 ng/ml of IL-6, 250 ng/ml of IL-6 soluble receptor (IL-6SR) or IL-6 (25 ng/ml)+IL-6SR (250 ng/ml).

FIG. 6B is a bar graph of the effect of monoclonal antibody against Il-6 on inhibition of GAG degradation. The Y axis represents the GAG concentration in μg/ml and the X axis represents the IL-6 monoclonal antibody in the following conditions: presence or absence of 25 ng/ml of IL-6, 250 ng/ml of L-6SR or IL-6 (25 ng/ml) plus IL-6SR (250 ng/ml).

FIG. 6C is a bar graph of the effect of monoclonal antibody against Il-6 on inhibition of NO production. The Y axis represents the NO concentration in μM and the X axis represents the IL-6 monoclonal antibody in the following conditions: presence or absence of 25 ng/ml of IL-6, 250 ng/ml of L-6SR or IL-6 (25 ng/ml)+IL-6SR (250 ng/ml).

FIG. 7A is a bar graph of the effect of JNJ 7583979 (RWJ 351958) on inhibition of Prostaglandin E₂ (PGE₂) synthesis. Alginate Recovered Chondrocyte (ARC) tissues were treated with JNJ 7583979 (RWJ 351958) at indicated concentrations (μM) in the presence of 10 ng/ml of IL-1β or absence of IL-1β. The Y axis represents PGE₂ levels in pg/ml and the X axis represents JNJ 7583979 (RWJ 351958) in μM. In addition, non-treated (NT) and Batimastat treated (Bat) tissues were tested as controls. Error bars on all graphs indicate standard error.

FIG. 7B is a bar graph of the effect of JNJ 7583979 (RWJ 351958) on inhibition of NO production. ARC tissues were treated with the test compound at indicated concentrations (μM) in the presence of 10 ng/ml of IL-1β or absence of IL-1β. The Y axis represents NO levels in μM and the X axis represents JNJ 7583979 (RWJ 351958) in μM. In addition, non-treated and Batimastat treated tissues were tested as controls.

FIG. 8 is a bar graph of the effect of JNJ 7583979 (RWJ 351958) on inhibition of glycosaminoglycan (GAG) release. ARC tissues were treated with the test compound at indicated concentrations in the presence of 10 ng/ml of IL-1β or absence of IL-1β. The Y axis represents GAG levels in μg/ml and the X axis represents JNJ 7583979 (RWJ 351958) in μM. In addition, non-treated and Batimastat treated tissues were tested as controls.

FIG. 9A-C are bar graphs of the effects of p38 MAP kinase inhibitors on inhibition of GAG degradation. Bovine ARC tissues were treated with test compounds at indicated concentrations (μM) in the absence (left bars) or presence (right bars) of 7.5 ng/ml of IL-1β. FIG. 9A is a bar graph of the effect of SCIO-282 (SD 282) on inhibition of GAG degradation. FIG. 9B is a bar graph of the effect of JNJ 17089540 (RWJ 669307) on inhibition of GAG degradation. FIG. 9C is a bar graph of the effect of JNJ 3026582 (RWJ 67657) on inhibition of GAG degradation. In FIG. 9A-C, the Y axis represents GAG levels in μg/ml and the X axis represents drug concentration in μM.

FIG. 10A-C are bar graphs of the cytotoxicity of p38 MAP kinase inhibitors. The Y axis represents LDH level (O.D. 492 nm) and the X axis represents the test compound in μM concentrations. The scales of Y axis were intentionally set at a larger value usually observed for a pellet with more than 80% of cell death to demonstrate the low toxicity of this class of molecules. In addition, non-treated (NT) and Batimastat (Bat) treated tissues were tested as controls. The same culture media was used as in FIGS. 7A-B, 8 and 9A-C. FIG. 10A is a bar graph of the cytotoxicity of SCIO-282 (SD 282) and JNJ 17089540 (RWJ 669307) in the presence of 10 ng/ml of Il-1β or absence of Il-1β. FIG. 10B is a bar graph of the cytotoxicity of JNJ 7583979 (RWJ 351958) in the presence of 10 ng/ml of Il-1β or absence of Il-1β. FIG. 10C is a bar graph of the cytotoxicity of JNJ 3026582 (RWJ 67657) in the presence of 10 ng/ml of Il-1β or absence of II-1β.

FIG. 11A is a bar graph of the effect of SCIO-469 (SD 469) on inhibition of GAG degradation in bovine ARC tissues stimulated with IL-1β in the presence of 10 ng/ml of IL-1β or absence of IL-1β. The Y axis represents GAG levels in μg/ml and the X axis represents SCIO-469 (SD 469) in nM concentrations in the presence or absence of IL-1β.

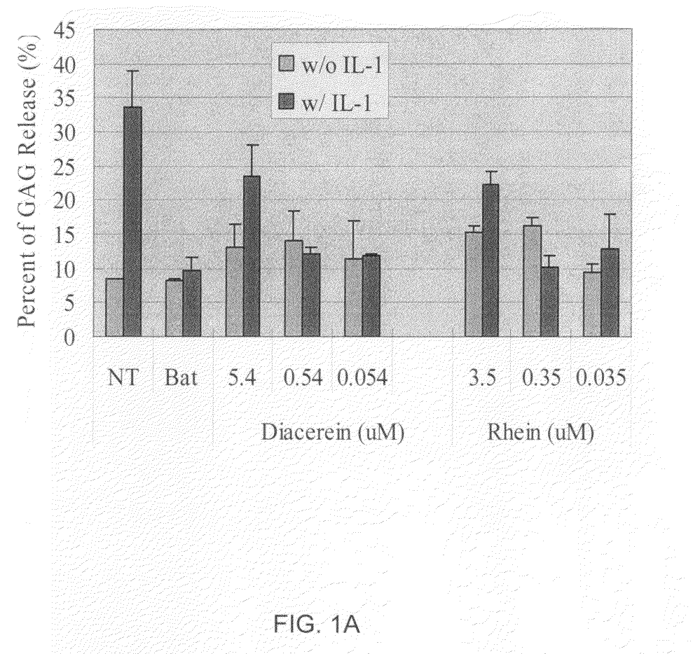

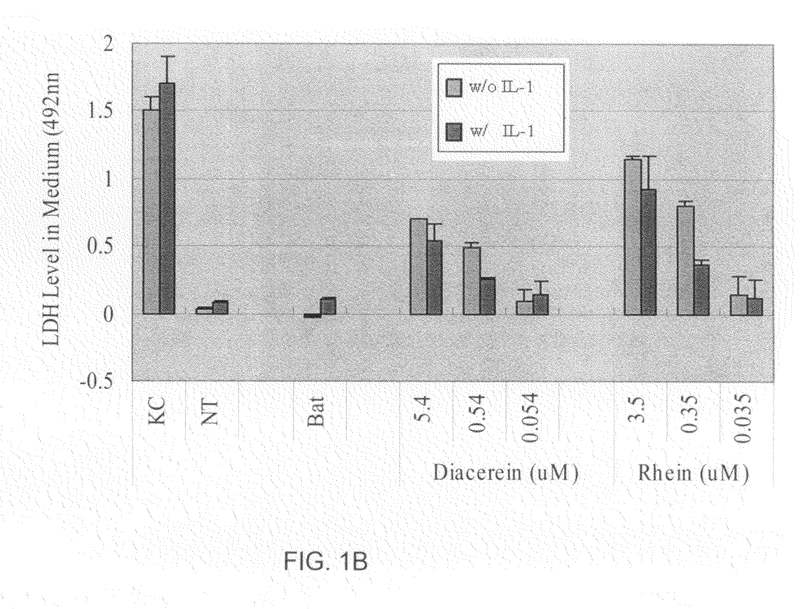

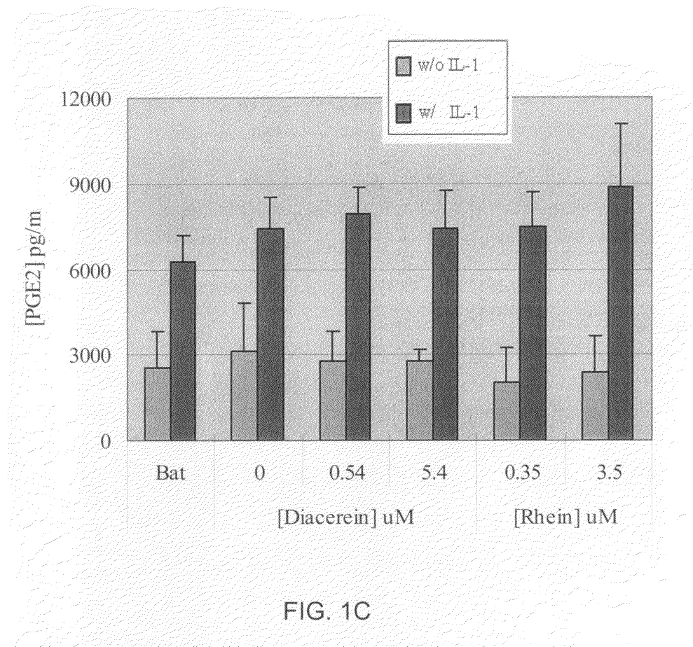

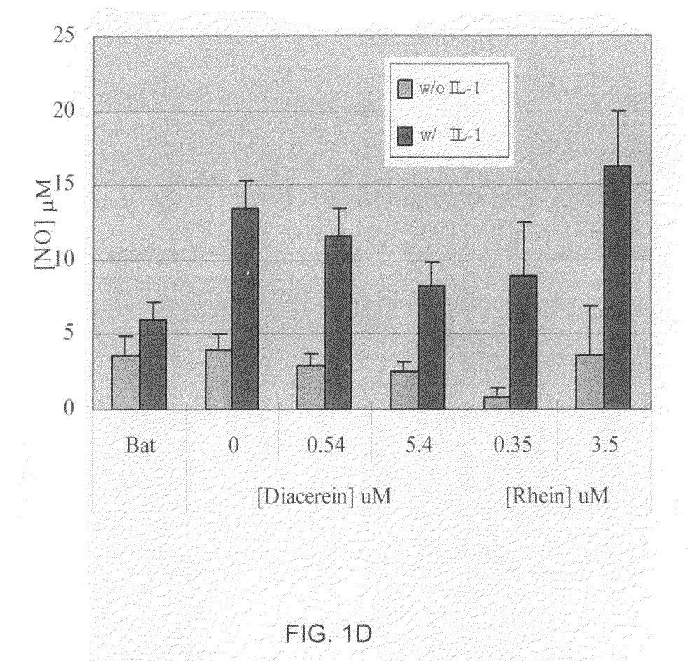

FIG. 1B is a bar graph of the effect of SCIO-469 (SD 469) on LDH level to assess cytotoxicity in bovine ARC tissues. The Y axis represents LDH levels in O.D. 492 nm and the X axis represents SCIO-469 (SD 469) in nM concentrations, in the presence of 10 ng/ml of 1′-1β or absence of IL-1β.

FIG. 12A is a dose-response curve of SCIO-469 (SD 469); SCIO-282 (SD 282); JNJ 3026582 (RWJ 67657); and JNJ 17089540 (RWJ 669307) for percent of inhibition of NO production in bovine ARC tissues in the presence of 10 ng/ml of Il-1β or absence of II-1β. FIG. 12B is a dose-response curve of SCIO-469 (SD 469); SCIO-282 (SD 282); JNJ 3026582 (RWJ 67657); and JNJ 17089540 (RWJ 669307) for percent of inhibition of PGE₂ synthesis. The Y axis represents percent of inhibition and the X axis represents the test compounds in μM concentrations.

FIG. 13A shows the chemical structures for JNJ 3026582 (RWJ 67657); JNJ 17089540 (RWJ 669307); and JNJ 7583979 (RWJ 351958).

FIG. 13B shows the chemical structure for JNJ 7706621.

FIG. 13C shows the chemical structure for SD-469.

FIG. 14A is a bar graph demonstrating the effects of diacerein and rhein on inhibition of GAG degradation in injured cartilage explants with stimulated or non-stimulated LPS PBMC. The Y axis represents percent GAG inhibition and the X axis represents diacerein and rhein in μM. In addition, non-injured (NI), non-treated (NT), Batimastat (Bat) treated data is shown.

FIG. 14B is a bar graph demonstrating the effects of diacerein and rhein on cytotoxicity in injured cartilage explants with stimulated or non-stimulated LPS PBMC. The Y axis represents percent LDH level (492 nm) and the X axis represents diacerein and rhein in μM. In addition, non-injured (NI), non-treated (NT), Batimastat (Bat) treated, and killed chondrocyte data are shown.

FIG. 15A is a bar graph demonstrating the effect of batimastat on inhibition of PGE₂ synthesis in injured cartilage explants in the presence or absence of a cocktail of cytokines (10 ng/ml of IL-1β, 100 ng/ml of TNF-α and 5 ng/ml of INF-γ) in the presence or absence of 10 μM of batimastat (Bat). The Y axis represents PGE₂ in pg/ml and the X axis represents batimastat (Bat) in μM. In addition, non-injured (NI) and non-treated (NT) data is shown.

FIG. 15B is a bar graph demonstrating the effect of batimastat on inhibition of NO production in injured cartilage explants in the presence or absence of a cocktail of cytokines (10 ng/ml of IL-1β, 100 ng/ml of TNF-α and 5 ng/ml of INF-γ) in the presence or absence of 10 μM of batimastat (Bat). The Y axis represents NO in μM and the X axis represents batimastat(Bat) in μM. In addition, non-injured (NI) and non-treated (NT) data is shown.

FIG. 16A is a bar graph demonstrating the effect of Rapamycin on inhibition of GAG degradation in injured cartilage explants with stimulated and non-stimulated LPS The Y axis represents percent of GAG inhibition. The X axis represents Rapamycin in μM. In addition, non-injured (NI) and Batimastat (Bat) treated data is shown.

FIG. 16B is a bar graph demonstrating the effect of Rapamycin on cytotoxicity in injured cartilage explants with stimulated and non-stimulated LPS. The Y axis represents LDH level on O.D. 492 nm. The X axis represents in μM. The X axis represents Rapamycin in μM. In addition, non-injured (NI), Batimastat (Bat) and killed chondrocytes (KC) treated data is shown.

FIG. 17A is a bar graph demonstrating the effect of JNJ 3026582 μM (RWJ 67657) on inhibition of GAG degradation in injured cartilage explants which were cultured in the presence (“+”) or absence (“−”) of inflammatory conditions (10 ng/ml of IL-1β, 100 ng/ml of TNFα and 5 ng/ml of IFN-γ). The Y axis represents GAG in μg/ml and the X axis represents JNJ 3026582 (RWJ 67657) in μM concentrations.

FIG. 17B is a bar graph demonstrating the effect of JNJ 3026582 (RWJ 67657) on inhibition of PGE₂ synthesis in injured cartilage explants which were cultured in the presence (“+”) or absence (“−”) of inflammatory conditions (10 ng/ml of IL-1β, 100 ng/ml of TNFα and 5 ng/ml of IFN-γ). The Y axis represents PGE₂ in pg/ml and the X axis represents JNJ 3026582 (RWJ 67657) in μM.

FIG. 17C is a bar graph demonstrating the effect of JNJ 3026582 (RWJ 67657) on inhibition of NO production in injured cartilage explants which were cultured in the presence (“+”) or absence (“−”) of inflammatory conditions (10 ng/ml of IL-1β, 100 ng/ml of TNFα and 5 ng/ml of IFN-γ). The Y axis represents NO in μM and the X axis represents JNJ 3026582 (RWJ 67657) in μM.

FIG. 17D is a bar graph demonstrating the effect of JNJ 7583979 (RWJ 351958) on inhibition of GAG release in injured cartilage explants with stimulated and non-stimulated LPS. The Y axis represents percent of GAG release and the X axis represents JNJ 7583979 (RWJ 351958) in μM. Data for “−LPS” not shown at 0.1, 1 and 10 due to sample contamination.

FIGS. 18A-F are bar graphs demonstrating the effects of p38 MAP kinase inhibitors on GAG degradation, NO production and PGE₂ synthesis in injured cartilage explants in the presence (“+”) or absence (“−”) of a cocktail of cytokines (10 ng/ml of IL-1β, 10 ng/ml IL-6 and 100 ng/ml of TNFα). “NI” is the non-injured cartilage specimen. FIG. 18A is a bar graph demonstrating the effect of JNJ 17089540 (RWJ 669307) on inhibition of GAG degradation. The Y axis represents GAG in μg/ml and the X axis represents JNJ 17089540 (RWJ 669307) in μM concentrations. FIG. 18B is a bar graph demonstrating the effect of SCIO-282 (SD 282) on inhibition of GAG degradation. The Y axis represents GAG in μg/ml and the X axis represents SCIO-282 (SD 282) in μM concentrations. FIG. 18C is a bar graph demonstrating the effect of JNJ 17089540 (RWJ 669307) on inhibition of NO production. The Y axis represents NO in μM and the X axis represents JNJ 17089540 (RWJ 669307) in μM. FIG. 18D is a bar graph demonstrating the effect of SCIO-282 (SD 282) on inhibition of NO production. The Y axis represents NO in μg/ml and the X axis represents SCIO-282 (SD 282) in μM concentrations. FIG. 18E is a bar graph demonstrating the effect of JNJ 17089540 (RWJ 669307) on inhibition of PGE₂ synthesis. The Y axis represents PGE₂ in pg/ml and the X axis represents JNJ 17089540 (RWJ 669307) in μM. FIG. 18F is a bar graph demonstrating the effect of SCIO-282 (SD 282) on inhibition of PGE₂ synthesis. The Y axis represents PGE₂ in pg/ml and the X axis represents SCIO-282 (SD 282) in μM concentrations.

FIGS. 19A-C are bar graphs demonstrating the effects of “complete”, DME/ITSx, GDF-5, p38 kinase inhibitor JNJ 3026582 (RWJ 67657), IL-1β, p38 kinase inhibitor JNJ 3026582 (RWJ 67657) plus GDF-5, Il-1β plus p38 kinase inhibitor JNJ 3026582 (RWJ 67657), Il-1β plus GDF-5, and Il-1β plus p38 kinase inhibitor JNJ 3026582 (RWJ 67657) and GDF-5 on GAG degradation in annulus fibrosus (AF) chondrocyte medium (FIG. 19A), nucleus pulposus (NP) chondrocyte medium (FIG. 19B), and annulus fibrosus chondrocyte medium (FIG. 19C). The Y-axis depicts measurement of average GAG content in culture media measured in pg/cell.

FIGS. 20A-C are bar graphs demonstrating the effects of DME/ITSx, GDF-5, p38 kinase inhibitor JNJ 3026582 (RWJ 67657), IL-1β, IL-1β and p38 MAP kinase inhibitor JNJ 3026582 (RWJ 67657), IL-1β plus GDF-5, Il-1β plus p38 MAP kinase inhibitor JNJ 3026582 (RWJ 67657) and GDF-5, p38 MAP kinase inhibitor JNJ 3026582 (RWJ 67657) and GDF-5, and “complete” on aggrecan (FIG. 20A), collagen type 1 (FIG. 20B) and collagen type 2 (FIG. 20C) relative mRNA for nucleus pulposus and annulus fibrosis cells. The Y-axis depicts relative mRNA content of the respective proteins.

FIGS. 21A-C are bar graphs demonstrating the effects of DME/ITSx, GDF-5, p38 kinase inhibitor JNJ 3026582 (RWJ 67657), IL-1β, IL-1β and p38 kinase MAP inhibitor (RWJ 67657), IL-1β plus GDF-5, Il-1β plus rhGDF-5 and p38 kinase MAP inhibitor JNJ 3026582 (RWJ 67657), and p38 MAP kinase inhibitor JNJ 3026582 (RWJ 67657) and GDF-5, and “complete” on MMP-3 (FIG. 21A), MMP-9 (FIG. 21B) and MMP-13 (FIG. 21C) relative mRNA for nucleus pulposus and annulus fibrosis cells. The Y-axis depicts relative mRNA content of the matrix metalloproteinases 3, 9, and 13.

The foregoing will be apparent from the following more particular description of example embodiments of the invention, as illustrated in the accompanying drawings.

### DETAILED DESCRIPTION OF THE INVENTION

A description of preferred embodiments of the invention follows.

For the purposes of the present invention, the terms “inhibitor” and “antagonist” are used interchangeably. A protein may be inhibited at, for example, the synthesis level, at the translation level, by shedding, by antibodies, or by soluble receptors. The term “patient” refers to a human having a degenerating or degenerated disc.

For the purposes of the present invention “transdiscal administration” includes, but is not limited to:
a) injecting a formulation into the nucleus pulposus of a degenerating disc, such as a relatively intact degenerating disc,
b) injecting a formulation into the annulus fibrosus of a degenerating disc, such as relatively intact degenerating disc,
c) providing a formulation in a patch attached to an outer wall of the annulus fibrosus,
d) providing a formulation in a depot at a location outside but closely closely adjacent to an outer wall of the annulus fibrosus (“trans-annular administration”), and
e) providing the formulation in a depot at a location outside but closely adjacent to an endplate of an adjacent vertebral body (“trans-endplate administration”).

Because DDD is a continuous process, the degenerating disc to which the therapeutic drug is administered may be in any one of a number of degenerative states. Accordingly, the degenerating disc may be an intact disc. The degenerating disc may be a herniated disc (wherein a portion of the annulus fibrosus has a bulge). The degenerating disc may be a ruptured disc (wherein the annulus fibrosus has ruptured and bulk nucleus pulposus has exuded). The degenerating disc may be delaminated (wherein adjacent layers of the annulus fibrosus have separated). The degenerating disc may have fissures (wherein the annulus fibrosus has fine cracks or tears through which selected molecules from the nucleus pulposus can leak).

Olmarker, Spine 26(8): 863-9 (2001) (“Olmarker I”) and Aoki, Spine 27(15): 1614-17 (2002), teach that TNF-α appears to play a role in producing the pain associated with the nucleus pulposus contacting nerve roots of the spinal cord.

U.S. Published Patent Application No. US 2003/0039651 (“Olmarker II”) teaches a therapeutic treatment of nerve disorders comprising administration of a therapeutically effective dosage of at least two substances selected from the group consisting of TNF inhibitors (both specific and non-specific), IL-1 inhibitors, IL-6 inhibitors, IL-8 inhibitors, FAS inhibitors, FAS ligand inhibitors, and IFN-gamma inhibitors.

In the examples of Olmarker II, Olmarker II further teaches that these substances are to be administered through systemic pathways. In particular, Olmarker II teaches that “the major contribution of TNF-alpha may be derived from recruited, aggregated and maybe even extravasated leukocytes, and that successful pharmacologic block may be achieved only by systemic treatment.” Of note, Olmarker II appears to discourage the local addition of one therapeutic agent (doxycycline) to a transplanted nucleus pulposus.

PCT Published Patent Application No. WO 02/100387 (“Olmarker III”) teaches the prevention of neovascularization and/or neo-innervation of intervertebral discs by the administration of anti-angiogenic substances. Again, however, Olmarker III teaches systemic administration of these therapeutic agents.

U.S. Pat. No. 6,419,944 (“Tobinick”) discloses treating herniated discs with cytokine antagonists, including REMICADE® infliximab. Tobinick teaches that local administration involves a subcutaneous injection near the spinal cord. Accordingly, Tobinick does not teach a procedure involving a sustained delivery of a drug for the treatment of DDD, nor directly administering a specific cytokine antagonist (such as infliximab) into the disc.

U.S. Published Patent Application No. 2003/0049256 (Tobinick II) discloses that injection of such therapeutic molecules to the anatomic area adjacent to the spine is accomplished by interspinous injection, and preferably is accomplished by injection through the skin in the anatomic area between two adjacent spinous processes of the vertebral column.

Tobinick II further teaches that TNF antagonists may be administered by interspinous injection in the human and that the dosage level is in the range of 1 mg to 300 mg per dose, with dosage intervals as short as two days. Tobinick II further discloses that Interleukin-1 antagonists are administered in a therapeutically effective dose, which will generally be 10 mg to 200 mg per dose, and their dosage interval will be as short as once daily.

Tobinick, Swiss Med Weekly, 133:170-77 (2003), (“Tobinick III”) teaches both perispinal and epidural administration of TNF inhibitors for spine related therapies.

Karppinen, Spine, 28(8):750-4 (2003), teaches intravenously injecting or orally administering infliximab into patients suffering from sciatica.

As with Tobinick I and II, Karppinen does not teach a procedure involving a sustained delivery of a drug for the treatment of DDD, nor directly administering a specific cytokine antagonist (such as REMICADE® infliximab) into the disc.

U.S. Pat. No. 6,352,557 (Ferree) teaches adding therapeutic substances such as anti-inflammatory medications to morselized extra-cellular matrix, and injecting that combination into an intervertebral disc.

However many anti-inflammatory agents are non-specific and therefore may produce unwanted side effects upon other cells, proteins and tissue. In addition, the pain-reducing effect of these agents is typically only temporary. Lastly, these agents typically only relieve pain, and are neither curative nor restorative.

Alini, Eur. Spine J. 11(Supp.2):S215-220 (2002), teaches therapies for early stage DDD, including injection of inhibitors of proteolytic enzymes or biological factors that stimulate cell metabolic activity (i.e., growth factors) in order to slow down the degenerative process. Alini I does not disclose inhibiting growth factors.

U.S. Published Patent Application US 2002/0026244 (“Trieu”) discloses an intervertebral disc nucleus comprising a hydrogel that may deliver desired pharmacological agents. Trieu teaches that these pharmacological agents may include growth factors such as TGF-B and anti-inflammatory drugs, including steroids. Trieu further teaches that these pharmacological agents may be dispersed within the hydrogel having an appropriate level of porosity to release the pharmacological agent at a desired rate. Trieu teaches that these agents may be released upon cyclic loading or upon resorption.

Takegami, Spine, 27(12):1318-25 (2000) teaches that injecting TGF-β into the disc space results in enhanced replenishment of the extracellular matrix damaged by cytokines. Takegami further teaches that the half-life of a growth factor injected into the interveterbal disc can be expected to be longer than that injected into a synovial joint because the nucleus pulposus is surrounded by the fibrous structure of the annulus fibrosus, thus providing a confined environment. Diwan, Tissue Engineering in Orthopedic Surgery, 31(3):453-464 (2000), reports on another Takegami paper that concluded that a delivery system allowing prolonged delivery (>3 days) would have to be used to obtain the observed effect of the growth factor.

Alini, Spine 28(5):446-54 (2003), discloses a cell seeded collagen-hyaluronan scaffold supplemented with growth factors such as TGF-B, bFGF, and IGF-1 for use in regenerating a nucleus pulposus.

Maeda et al. Spine 25(2): 166-169 (2000) reports on the in vitro response to interleukin-1 receptor antagonist protein (IRAP) of rabbit annulus fibrosus exposed to IL-1. Maeda suggests that IRAP could be useful in inhibiting the degradation of the disc.

Yabuki, Spine, 26(8):870-5 (2001), teaches the use of an anti-TNF drug for the treatment of sciatica.

U.S. Pat. No. 6,277,969 (“Le”) discloses the use of anti-TNF antibodies for therapy of TNF-mediated pathologies. Le teaches parenteral administration of the antibodies.

Ariga, Spine, 28(14):1528-33 (2003), reports that the application of a p38 MAP kinase inhibitor to a culture of organ-cultured intervertebral discs increased the occurrence of apoptosis in endplate chondrocytes in the culture.

The present invention is directed to providing directly through a diseased intervertebral disc at least one cytokine antagonist capable of inhibiting a cytokine (e.g., a pro-inflammatory cytokine) present in the disc. In some embodiments, the antagonist is a small molecule inhibitor of p38 MAP kinase. In one embodiment, the cytokine antagonist inhibits the action of a p38 MAP kinase released by disc cells or by invading macrophages during the degenerative process. In one embodiment, the cytokine antagonist inhibits the action of a p38 MAP kinase released by disc cells or by invading macrophages during the degenerative process. In some embodiments, the cytokine antagonist is a p38 MAP kinase inhibitor.

In the instant specification, p38, p38 MAP kinase and p38 MAP kinase are used interchangeably.

In some embodiments, the antagonist is specific to two cytokines. In some embodiments, the antagonist is specific to one cytokine.

In some embodiments, the antagonist is capable of inhibiting a pro-inflammatory cytokine selected from the group consisting of TNF-α, an interleukin (preferably IL-1, IL-6 and IL-8), FAS, an FAS ligand, and IFN-gamma. Such inhibitors include those identified on pages 5-18 of Olmarker II, supra, (U.S. Published Patent Application No.: 2003/0039651) the specification of which is incorporated herein by reference in its entirety.

In some embodiments, the cytokine antagonist inhibits the cytokine (such as p38 MAP kinase) by preventing its production. In some embodiments, the cytokine antagonist inhibits the cytokine by binding to a membrane-bound cytokine. In others, the cytokine antagonist inhibits the cytokine by binding to a solubilized, e.g. soluble, cytokine. In some embodiments, the cytokine antagonist inhibits the cytokine by both binding to membrane bound cytokines and to solubilized cytokines. In some embodiments, the cytokine antagonist is a monoclonal antibody (“mAb”). The use of mAbs is highly desirable since they can bind specifically to a certain target protein and to no other proteins. In some embodiments, the cytokine antagonist inhibits the cytokine by binding to a natural receptor of the target cytokine. In some embodiments, the cytokine antagonist is a small molecule.

In some embodiments, the cytokine antagonist (e.g., a p38 MAP kinase inhibitor) is an antagonist (e.g., a specific antagonist) of an interleukin. Preferably, the target interleukin is selected from the group consisting IL-1, IL-2, IL-6 and IL-8, and IL-12. In some embodiments, the interleukin is IL-1β. Preferred antagonists include but are not limited to Kineretg (recombinant IL-1-RA, Amgen), IL-1-Receptor Type 2 (Amgen) and IL-1 Trap (Regeneron).

### p38 Map Kinase Inhibitors

Transdiscal administration of an effective amount of an antagonist of p38 MAP kinase would also help provide therapy to a patient having DDD. Therefore, in accordance with another embodiment of the present invention, there is provided a method of treating degenerative disc disease in an intervertebral disc having a nucleus pulposus, comprising transdiscally administering an effective amount of a formulation comprising an antagonist of p38 MAP kinase into an intervertebral disc.

The inhibition of p38 MAP kinase is believed to inhibit (e.g., block production) of pro-inflammatory cytokines such as TNF-α. p38 MAP kinase inhibitors also inhibit production of IL-1, IL-6 and IL-8, but not IL-2. Without wishing to be tied to a theory, it is believed that inhibition of p38 MAP kinase should not block TGF signaling nor TGF activity. p38 MAP kinase inhibitors may also block induction of some metalloproteinases, COX2 and NO synthetase, but they do not inhibit interleukins involved in immune cell proliferation such as IL-2. Both p38 MAP kinases and JNK MAP kinases play important roles in mediating proinflammatory cytokine stimulation and synthesis, for cytokines such as interleukin 1 and tumour necrosis factor alpha, maintaining the chronicity of chondrocyte anabolism and catabolism and mediating tissue damage. The availability of potent and selective p38 mitogen activated protein kinase inhibitors provides a means for further dissecting the pathways implicated in cytokine production.

In some embodiments, the antagonist is a small molecule inhibitor of p38 MAP kinase. The small molecule inhibitors of p38 MAP kinase are potent (˜nM). Preferably, they are provided in a 5-100 microgram dose. In one embodiment, the inhibitors are provided in a dose (or doses) of about 25-about 100 mg/kg. For in vitro assays, the “dose” can be 5-100 nM (nanomolar). In some embodiments, the dose can be about 0.02-50 μM (micromolar). For in vitro assays, the “dose” is usually 5-100 nM (nano-molar). For an in vivo dose, the dose is about 1 cc. In some embodiment, the p38 MAP kinase inhibitor is present in the formulation in an amount of at least about 1 microgram/ml to about 5 milligram/ml, for example, about 5 mg/ml. In some embodiments, the range is about 1 μg/ml-1.2 mg/ml. Some antagonists of p38 MAP kinase are disclosed in Zhang, J. Biol. Chem., 272(20): 13397-402 (May 16, 1997); Pargellis, Nature Structural Biology, 9(4): 268-272 (April 2002); and Chae, Bone, 28(1): 45-53 (January 2001), and in U.S. Pat. No. 6,541,477 (“Goehring”) and U.S. Pat. No. 5,965,583 (“Beers”) and U.S. Pat. No. 7,244,441 (“Schreiner”), the contents of which are hereby incorporated by reference in their entirety.

In some embodiments, the p38 MAP kinase inhibitor is selected from the group consisting of JNJ 7583979 (RWJ 351958), JNJ 3026582 (RWJ 67657), JNJ 17089540 (RWJ 669307), SCIO-469 (SD 469) (Scios) and SCIO-282 (SD 282) (Scios).

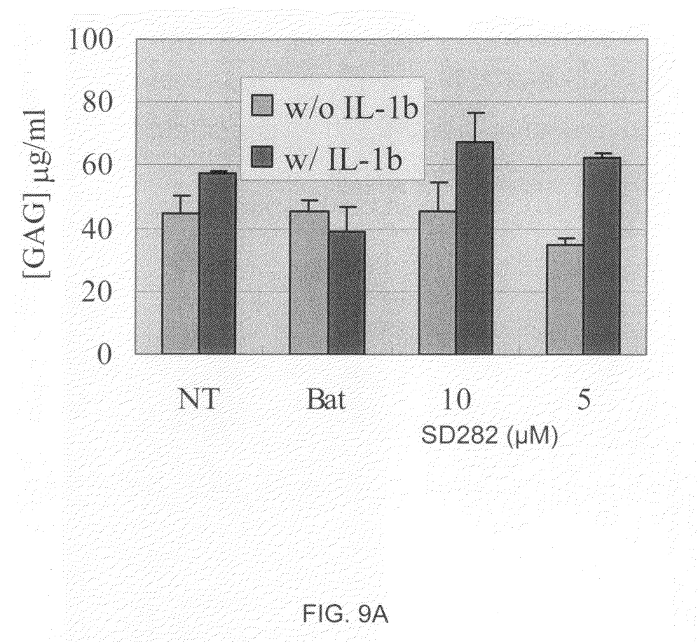

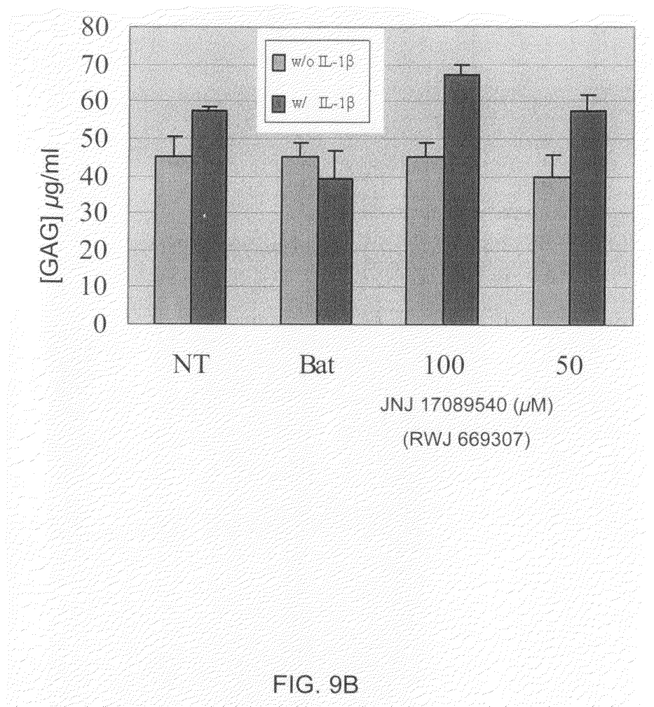

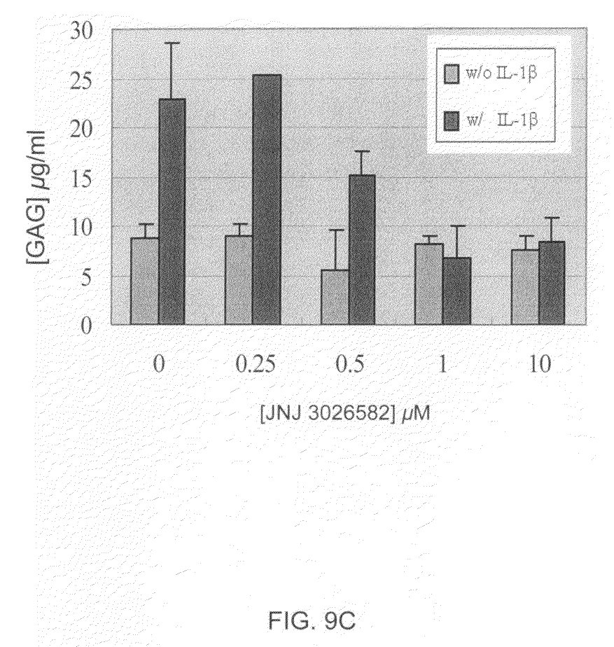

JNJ 3026582 (RWJ 67657) has the following properties: inhibitor of p38a MAP kinase with an IC₅₀ 20 nM; binds p38a MAP kinase with a Kd of 9 nM; inhibitor of JNK2 with an IC₅₀ 80 nM; inhibitor of LPS-induced TNF human peripheral blood mononuclear cells (hPBMC) with an IC₅₀ 4 nM; inhibitor of COX1 with an IC₅₀>10 μM; and inhibitor of COX2 with an IC₅₀>10 μM; molecular weight 425.51; poor water solubility (soluble in 0.1 M HCl); and soluble at pH<2. JNJ 3026582 (RWJ 67657) is a potent dual inhibitor of both p38 MAP kinase and c-Jun N-terminal kinase (JNK MAP kinase). The structure of JNJ 3026582 (RWJ 67657) is represented in FIG. 13A. As seen in Example III, JNJ 3026582 (RWJ 67657) significantly inhibited GAG degradation (FIG. 9C) and inhibited NO production and PGE₂ synthesis (FIGS. 12A and 12B and Table 3B) in a chondrocyte pellet culture model. As seen in Example IV, JNJ 3026582 (RWJ 67657) dose-dependently inhibited NO production and PGE₂ synthesis (FIGS. 17B and 17C and Table 4) in a drop tower model. As seen in Example V, it prevented GAG release in a dose dependent manner. JNJ 3026582 (RWJ 67657) is a 2-ethylynyl imidazole derivative.

JNJ 17089540 (RWJ 669307) has the following properties: inhibitor of p38a MAP kinase with an IC₅₀ 2 μM; binds to inactivated p38 MAP kinase with a Kd 100 nM; inhibitor of LPS-induced TNF hPBMC with an IC₅₀ 220 nM; inhibitor of LPS-induced IL-1 in hPBMC with an IC₅₀ of 250 nM; and inhibitor of COX1 and COX2 with an IC₅₀>10 μM; molecular weight 403.46; and water solubility >100 mg/ml of HCl salt. JNJ 17089540 (RWJ 669307) is a weak inhibitor of p38 MAP kinase and it is a TNFα modulator. The term “modulator” of TNFα means that it prevents TNFα from becoming activated. Its mechanism of action is based on binding to unactivated p38 MAP kinase and preventing its phosphorylation activity. JNJ 17089540 (RWJ 669307) does not inhibit active p38 MAP kinase directly, but rather binds to unactivated p38 MAP kinase and prevents its activation by upstream kinases. The structure of JNJ 17089540 (RWJ 669307) is represented in FIG. 13A. As seen in Example IV, JNJ 17089540 (RWJ 669307) dose-dependently inhibited NO production and PGE₂ synthesis (FIGS. 18C and 18E) in a drop tower model. JNJ 17089540 is a 3,4 aryl or heteroaryl substituted pyrrole derivative.

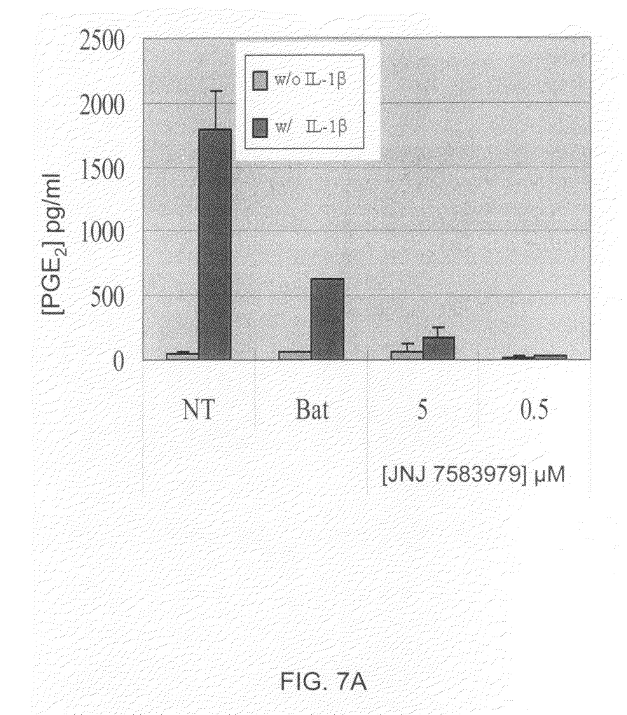

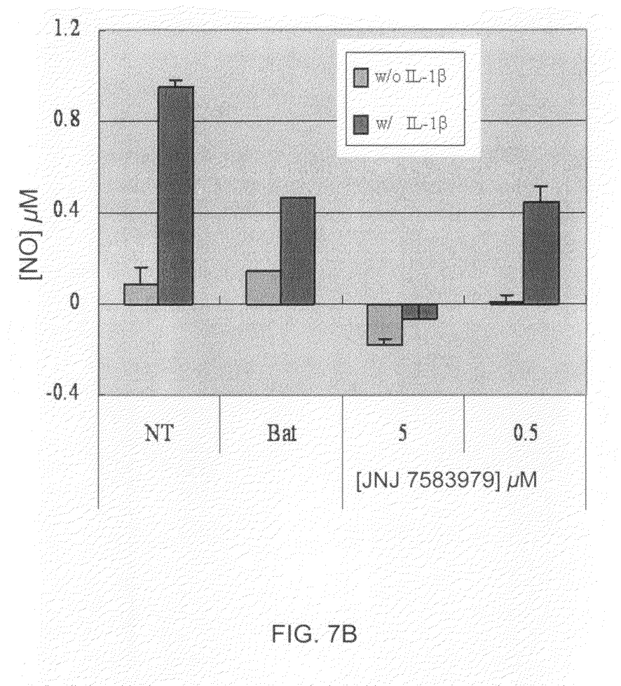

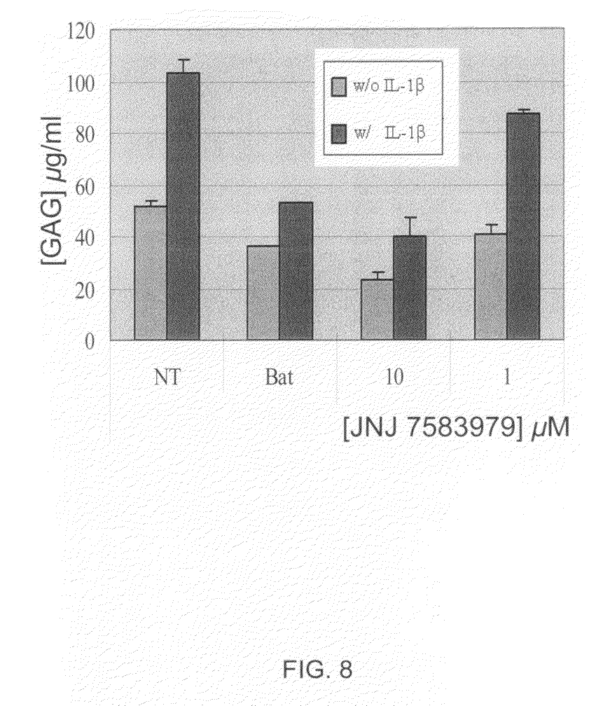

JNJ 7583979 (RWJ 351958) has the following properties: inhibitor of p38a MAP kinase with an IC₅₀ 1 nM; inhibitor of JNK with an IC₅₀ 12 nM; inhibitor of TNF human whole blood (HWB) with an IC₅₀ 5 nM; inhibitor of COX1 and COX2 with an IC₅₀>10 μM; molecular weight 425.47; and poor water solubility (>1 mg/ml at pH 2). The structure of JNJ 7583979 (RWJ 351958) is represented in FIG. 13A. As seen in Example III, JNJ 7583979 (RWJ 351958) significantly inhibited NO production and PGE₂ synthesis (FIGS. 7A and 7B and Table 3B) and significantly inhibited GAG degradation (FIG. 8) in a chrondrocyte pellet culture model. JNJ 7583979 is a 2,3 aryl or heteroaryl substituted imidazopyrimidine derivative.

SCIO-282 (SD 282) has the following properties: inhibitor of p38a MAP kinase with an IC₅₀ 1.6 nM; inhibitor of p38β with an IC₅₀ of 23 nM; selectivity for p38a MAP kinase and p38β MAP kinase of approximately 15; CYP2C9 with an IC₅₀ of 0.5 μM; inhibits TNFα h-WBA (EC50), of 0.07 μM (10× diluted); Rat (F) PK (F %,t1/2) of 46%; and limited water solubility. SCIO-282 (SD 282) is a direct inhibitor or selective inhibitor of p38 MAP kinase alpha and it has limited water solubility. As seen in Example III, SCIO-282 (SD 282) inhibited both NO production and PGE₂ synthesis (FIGS. 12A and 12B) in a chrondrocyte pellet culture model. As seen in Example IV, SCIO-282 (SD 282) dose-dependently inhibited NO production and PGE₂ synthesis (FIGS. 18D and 18F) in a drop tower model. SD-282 has the identical chemical structure as SD-469 except that the indole nitrogen has a hydrogen instead of a methyl substituent.

SCIO-469 (SD 469) has the following properties: inhibitor of p38α MAP kinase with an IC₅₀ 9 nM; inhibitor of p38β MAP kinase with an IC₅₀ of 98 nM; selectivity for p38a MAP kinase and p38β MAP kinase of approximately 10; 6 CYP450 with an IC₅₀ of >1 μM; inhibits TNFα h-WBA (EC50), of 1.6 μM (10× diluted); Rat (F) PK (F %,t1/2) of 1 h; Rat (M) PK (F %,t1/2) of 15%, 0.5 h; Monkey (F) PK (F %,t1/2) of 12%, 1.3 h; and Monkey (F) PK (F %,t1/2) of 12%, 1.3 h; formula C₂₇H₃₀N₄O₃F₁Cl₁; molecular weight is 513.01; molecular volume (cm³) is 273.3; surface area (cm²/mol×10⁹) is 33.84; Log P is 2.82; molar volume (cm³/mol) is 356.8; HLB is 11.55; Hansen's solubility parameter (delta/sqr (Mpa)) is 23.9 (11.72H); % hydrophilic surface is 48.8; water solubility (mg/mL) is 0.00004; H bond acceptor is 0.95; H bond donor is 0.43; polarity is 8.15; dipole moment (debyes) is 4.89; and max charge on N is −0.40226. SCIO-469 (SD 469) is a p38 MAP kinase inhibitor specific for the [symbol] unit. As seen in Example IV, SCIO-469 (SD 469) inhibited NO production and PGE₂ synthesis (FIGS. 12A and 12B) in a drop tower model. The structure of SD-469 is represented in FIG. 13C.

SCIO-282 and SCIO-469 are indole based heterocyclic inhibitors. Mavunkel, B., et al., “Indole-Based Heterocyclic Inhibitors of p38 MAP Kinase: Designing a Conformationally Restricted Analogue,” Bioorganic & Medicinal Chemistry Letters, 13: 3087-3090 (2003) is incorporated herein by reference in its entirety.

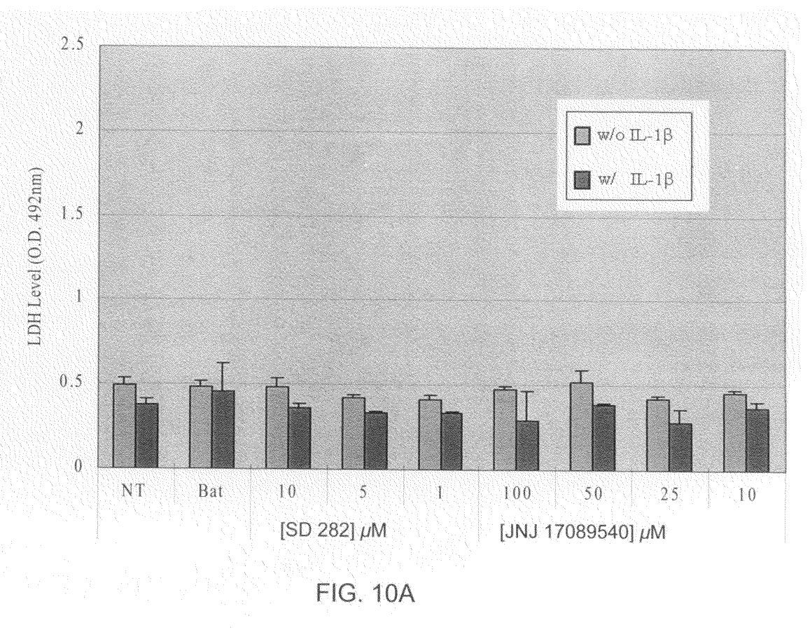

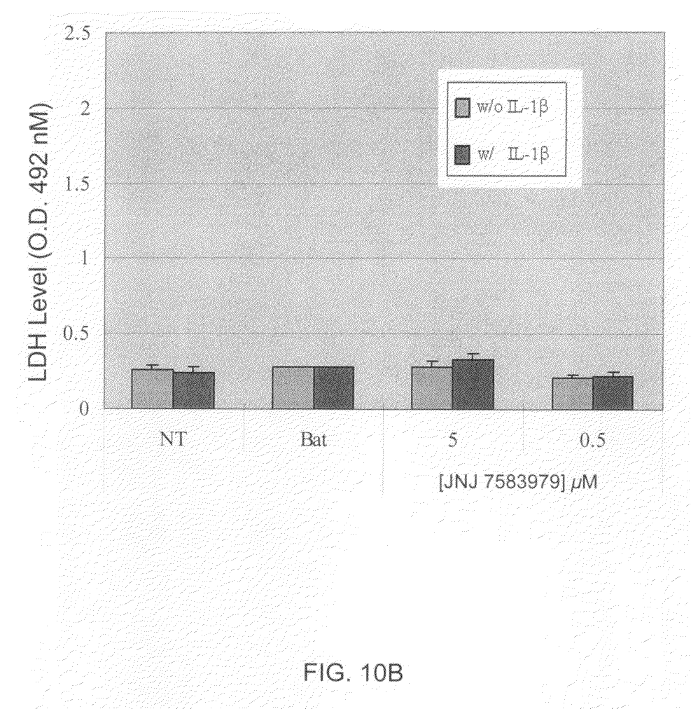

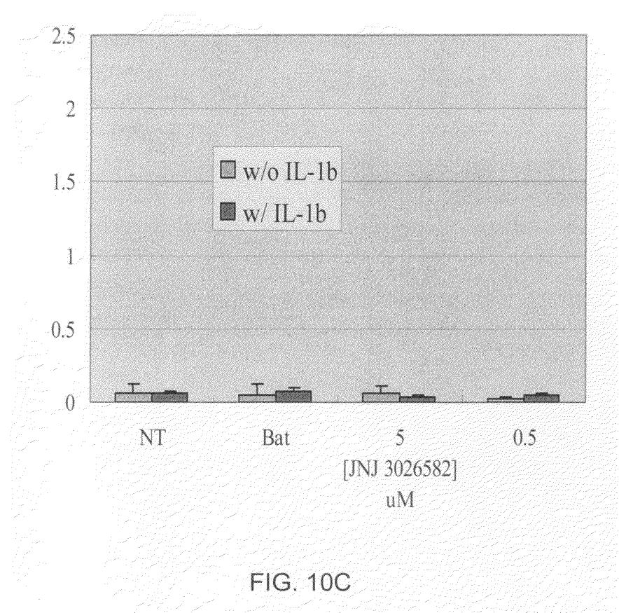

Further, as seen in Example III, FIGS. 10A-C, JNJ 7583979 (RWJ 351958), JNJ 3026582 (RWJ 67657), JNJ 17089540 (RWJ 669307) and SD282 all showed little cytoxicity to chondrocytes within the tested concentrations.

The JNJ 7583979 (RWJ 351958), JNJ 3026582 (RWJ 67657), and JNJ 17089540 (RWJ 669307) compounds are Aryl-pyridyl heterocycles. The SCIO-282 and SCIO-469 compounds are indol-5-carboxamides.

In some embodiments, the p38 MAP kinase inhibitor is selected from the group consisting of:
a) diaryl imidizole;
b) N,N′-diaryl urea (developed by Bayer, Boehringer Ingelheim and Vertex);
c) N,N-diaryl urea (developed by Vertex);
   d) benzophenone (developed by Leo Pharmaceuticals);
   e) pyrazole ketone (developed by Hoffman-LaRoche);
   f) indole amide (developed by GlaxoSmithKline and Scios);
   g) diamides (developed by AstraZeneca);
   h) quinazoline (developed by GlaxoSmithKline);
   i) pyrimido [4,5-d]pyrimidinone (developed by GlaxoSmithKline and Hoffman LaRoche); and
   j) pyridylamino-quinazolines (developed by Scios).

Members of this group are described, for example, in Zhang et al., supra, Pargellis et al., supra, Chae et al., supra, and Cirillo et al., Current Topics in Medicinal Chemistry, 2: 1021-1035 (2002), Boehm et al., Exp. Opin, Ther. Patents, 10(1):25-38 (2000), and Lee et al., Immunopharmacology, 47: 185-2001 (2000).

In some embodiments, the p38 MAP kinase inhibitor is characterized as a 1-aryl-2-pyridinyl heterocycle. In some embodiments, the 1-aryl-2-pyridinyl heterocycle is selected from the group consisting of:
a) 4,5 substituted imidazole,
b) 1,4,5 substituted imidizole;
c) 2,4,5 substituted imidizole;
d) 1,2,4,5 substituted imidizole; and
e) non-imidizole 5-membered ring heterocycle.

In some embodiments, the p38 MAP kinase inhibitor has at least 3 cyclic groups.

In some embodiments, the p38 MAP kinase inhibitor is selected from the group consisting of a molecule that is readily soluble in water and a substantially water insoluble molecule. In some embodiments, the p38 MAP kinase inhibitor is a substantially water insoluble molecule. The substantially water insoluble p38 MAP kinase inhibitor may be advantageous in that, if injected into the nucleus pulposus, it will remain in the nucleus pulposus as a solid and only slightly solubize over time, thereby providing sustained release.

In some embodiments, the p38 MAP kinase inhibitor is selected from the group consisting of:
a) SK&F 86002;
b) SB 203580;
c) L-167307;
d) HEP 689;
e) SB220025;
f) VX-745;
g) SU4984;
h) RWJ 68354;
i) ZM336372;
j) PD098059;
k) SB235699;
l) SB220025;
m) JNJ 3026582 (RWJ 67657) (Johnson & Johnson);
n) JNJ 17089540 (RWJ 669307) (Johnson & Johnson);
o) JNJ 7583979 (RWJ 351958) (Johnson & Johnson);
p) SCIO-282 (SD 282) (developed by Scios); and
q) SCIO-469 (SD 469) (developed by Scios).

Additional details regarding these inhibitors are found in Table 1.

TABLE 1
P38 MAP Kinase Inhibitors

| Name | Chemical Formula |
|---|---|
| JNJ3026582 (RWJ | 4-[4-(4-fluorophenyl)-1-(3-phenylpropyl)-5-(4-pyridinyl)-1H- |
| 67657) | imidazol-2-yl]-3-butyn-1-ol |
| SK&F86002 | 6-(4′-fluorophenyl)-5-(4′-pyridyl)-2,3-dihydroimadazo(2,1-b)- |
| | thiazole |
| SB203580 | 4-(4-fluorophenyl)-2-(4-methylsulfinylphenyl)-5-(4- |
| | pyridyl)imidazole |
| L-167307 | 3-(4-pyridyl-2-(4-fluoro-phenyl)-5-(4-methylsulfinylphenyl)- |
| | pyrrole |
| HEP689 | Aminobenzophenone compound |
| SB220025 | 5-(2amino-4-pyrimidinyl)-4-(4-fluorophenyl)-1-(4- |
| | piperidinyl)imidazole |
| VX-745 | 5-(2,6-Dichlorophenyl)-2-(phenylthio)-6H-pyrimido[1,6- |
| | b]pyridazin-6-one |
| SU4984 | 4-[4-(2-Oxo-1,2-dihydro-indol-3-ylidenemethyl)-phenyl]- |
| | piperazine-1-carbaldehyde |
| RWJ-68354 | 6-Amino-2-(4-fluorophenyl)-4-methoxy-3-(4pyridyl)-1H- |
| | pyrrolo[2,3-b]pyridine |
| ZM336372 | N-[5-(dimethyl-aminobensamido)-2-methylphenyl]-4- |
| | hydroxybenzamide |
| PD098059 | 2′-Amino-3′-methoxyflavone |
| SB220025 | 5-(2-Amino-4-pyrimidinyl)-4-(fluorophenyl)-1-(4- |
| | piperidinyl)imidazole |
| PD169316 | 4-(4-Fluorophenyl)-2-(4-nitrophenyl)-5-(4-pyridyl)-1H-imidazole |
| ML3403 | (RS)-{4-[5-(4-Fluorophenyl)-2-methylsulfanyl-3H-imidazol-4- |
| | yl]pyridin-2-yl}-(1-phenylethyl)amine] |
| ML3163 | 4-[5-(4-Fluorophenyl)-2-(4-methanesulfinyl-benzylsulfanyl)-3H- |
| | imidazol-4-yl]pyridine |
| SB242235 | 1-(4-piperidinyl)-4-(4-fluorophenyl)-5-(2-methoxy-4-pyrimidinyl) |
| | imidazole |
| SB239063 | trans-1-(4-Hydroxycyclohexyl)-4-(4-fluorophenyl)-5-(2- |
| | methoxypyrimidin-4-yl)imidazole |
| M39 | Aminopyridine-based inhibitor |
| SD-169 | Indole-5-carboxamide |
| EO-1428 | |
| SC-68376 | 2-Methyl-4-phenyl-5-(4-pyridyl)oxazole |
| PD169316 | 4-(4-Fluorophenyl)-2-(4-nitrophenyl)-5-(4-pyridyl)-1H-imidazole |
| SB202190 | 4-(4-Fluorophenyl)-2-(4-hydroxyphenyl)-5-(4-pyridyl)1H- |
| | imidazole |
| TAK-715 | N-[4-[2-ethyl-4-(3-methylphenyl)-1,3-thiazol-5-yl]-2- |
| | pyridyl]benzamide |
| VX-702 | |
| RPR 200765A | A 2-(2-dioxanyl)imidazole |
| BIRB-796 | Non-diaryl imidazole inhibitor |
| AMG-548 | Structure undisclosed |
| Ro-320-1195 | S-[5-Amino-1-(4-fluorophenyl)-1H-pyrazol-4-yl]-[3-(2,3- |
| | dihydroxypropoxy)phenyl]methanone |

### Tumor Necrosis Factor Inhibitors

In some embodiments, the agent is a highly specific inhibitor of TNF-α. In some embodiments, the p38 MAP kinase inhibitor is a TNF-α inhibitor. In some embodiments, the inhibitor of TNF-α is an inhibitor of p38 MAP kinase, preferably, a small molecule inhibitor of p38 MAP kinase.

In some embodiments, the TNF-α inhibitor inhibits the TNF-α by binding to membrane-bound TNF-α in order to prevent its release from membrane. In others, the TNF-α inhibitor inhibits the TNF-α by binding to solubilized TNF-α. One example thereof is etanercept. In some embodiments, the TNF-α inhibitor inhibits the TNF-α by both binding to membrane bound TNF-α and to solubilized TNF-α. One example thereof is REMICADE® infliximab. In some embodiments, the TNF-α inhibitor inhibits the TNF-α by preventing its production. In some embodiments, the cytokine antagonist inhibits the cytokine (e.g., TNF-α) by binding to a natural receptor of the target cytokine. In some embodiments, the TNF-α inhibitor is an inhibitor of TNF-α synthesis.

Preferred TNF antagonists include, but are not limited to, the following: etanercept (ENBREL®, Amgen); infliximab (REMICADE®, Johnson & Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and onercept, a recombinant TNF binding protein (r-TBP-1) (Serono); and CNTO 148 (Centocor/Johnson & Johnson), which is a fully human antibody and disclosed in U.S. Pat. No. 7,250,165 the contents of which is hereby incorporated by reference in its entirety.

TNF antagonists suitable for compositions, combination therapy, co-administration, devices and/or methods of the present invention (optionally further comprising at least one antibody, specified portion and/or variant thereof, of the present invention), include, but are not limited to, anti-TNF antibodies (e.g., at least one TNF antagonist (e.g., but not limited to a TNF chemical or protein antagonist, e.g., an anti-TNF antibody, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, REMICADE® infliximab, ENBREL® etanercept, HUMIRA™ adalimulab, CDP-571, CDP-870, afelimomab, lenercept, and the like); antigen-binding fragments thereof, receptor molecules which bind specifically to TNF; compounds which prevent and/or inhibit TNF synthesis, TNF release or its action on target cells, such as thalidomide, tenidap, phosphodiesterase inhibitors (e.g. pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody”, “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNF-alpha (TNFα). A suitable TNF antibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

In one embodiment, the antagonist is REMICADE®, infliximab, or cA2. Chimeric monoclonal antibody cA2 consists of the antigen binding variable region of the high-specificity neutralizing mouse anti-human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of cA2 is derived from the variable region of A2. A preferred source for nucleic acids encoding the variable region of the murine antibody A2 is the A2 hybridoma cell line designated c134A. Chimeric antibody cA2 is produced by a cell line designated c168A.

Chimeric A2 (cA2) neutralizes the cytotoxic effect of both natural and recombinant human TNFα in a dose dependent manner. Preferred methods for determining monoclonal antibody specificity and specificity by competitive inhibition can be found in Harlow et al., Antibodies. A Laboratory Manual, Cold Spring Harbor Laboratory Press, Cold Spring Harbor, N.Y. (1988); Colligan et al., eds., Current Protocols in Immunolog, Greene Publishing Assoc. and Wiley Interscience, New York, (1992-2000); Kozbor et al., Immunol. Today, 4:72-79 (1983); Ausubel et al., eds. Current Protocols in Molecular Biology, Wiley Interscience, New York (1987-2000); and Muller, Meth. Enzymol., 92:589-601 (1983), which references are entirely incorporated herein by reference.

In one embodiment, the TNF antibody is selected from the group of compounds disclosed in U.S. Pat. No. 6,277,969, the specification of which is entirely incorporated by reference. In some embodiments, the TNF antibody is delivered in a formulation having an antibody concentration of between about 30 mg/ml and about 60 mg/ml.

In some embodiments, the antibody binds to human TNFα with an affinity of 1×10⁸ liter/mole, measured as an association constant (Ka). In some embodiments, the affinity constant of the antibody is 1.0⁴×10¹⁰ M⁻¹. Additional examples of monoclonal anti-TNF antibodies that can be used in the present invention are described in the art (see, e.g., U.S. Pat. No. 5,231,024; Möoller, A. et al., Cytokine 2(3):162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

Preferred TNF receptor molecules useful in the present invention are those that bind TNFα with high specificity (see, e.g., Feldmann et al., International Publication No. WO 92/07076 (published Apr. 30, 1992); Schall et al., Cell, 61:361-370 (1990); and Loetscher et al., Cell, 61:351-359 (1990), which references are entirely incorporated herein by reference) and, optionally, possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high specificity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of a TNF receptor molecule refers to the portion of the TNF receptor molecule, or the portion of the TNF receptor molecule sequence which encodes the TNF receptor molecule, that is of sufficient size and sequences to functionally resemble TNF receptor molecules that can be used in the present invention (e.g., bind TNFα with high specificity and possess low immunogenicity). A functional equivalent of a TNF receptor molecule also includes modified TNF receptor molecules that functionally resemble TNF receptor molecules that can be used in the present invention (e.g., bind TNFα with high specificity and possess low immunogenicity). For example, a functional equivalent of a TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2003).

In some embodiments, the monoclonal antibody that inhibits a cytokine (e.g., TNF-α) is selected from the group consisting of monoclonal rodent-human antibodies, rodent antibodies, human antibodies or any portions thereof, having at least one antigen binding region of an immunoglobulin variable region, which antibody binds TNF. In one embodiment, this monoclonal antibody is selected from the group of compounds disclosed in U.S. Pat. No. 6,277,969, the specification of which is entirely incorporated by reference. In some embodiments, the REMICADE® infliximab is delivered in a formulation having an infliximab concentration of between about 30 mg/ml and about 60 mg/ml.

DDD involves the progressive degeneration of a disc in which many factors are involved. In many instances, simply providing a single dose or even a regimen over the space of a few days may not be sufficient to resolve the DDD. For example, if DDD were caused in part by mechanical instability in a functional spinal unit, then simply providing a one-time therapy for the disc cells would likely only delay the onset of the DDD. Therefore, there is a need to provide a long-term drug therapy treatment of DDD that does not require multiple injections.

Because the cytokines of interest both produce pain and degrade the ECM when present within the nucleus pulposus, it is desirable for the cytokine antagonist to remain within the nucleus pulposus as long as possible in a pharmaceutically effective amount. The half-life of the cytokine antagonist within the nucleus pulposus will depend upon many factors, including the size of the cytokine antagonist and its charge. In general, the larger the molecular weight of the cytokine antagonist, the more likely it is to remain contained by the annulus fibrosus portion of the disc.

If the half-life of the cytokine antagonist is relatively short, then it would be desirable for a relatively large dose of the cytokine antagonist to be administered into the disc. In this condition, quick depletion of the cytokine antagonist would not cause the cytokine antagonist to fall below therapeutically effective levels until an extended period.

Although a large dose of the cytokine antagonist would be desirable in such instances, injecting a critical volume of water can increase pressure in the nucleus pulposus. Nociceptors present on the inner wall of the annulus fibrosus react to this increased pressure and produce pain. One avenue for increasing the pressure in the nucleus pulposus is to inject a critical volume of water. In some cases, the added amount could be as little as one cc by volume to produce pain. Accordingly, if a dilute concentration of a cytokine antagonist is added to the nucleus pulposus to provide a large dose, the resulting pressure increase caused by this added volume could be sufficient to cause acute pain.

For example, if it were determined that about 100 mg of an cytokine antagonist was needed to therapeutically affect a nucleus pulposus, and that cytokine antagonist was provided in concentrations of from about 30-60 mg/ml, then at least about 1.5 ml of the cytokine antagonist should be injected into the nucleus pulposus in order to provide the desired therapeutic effect. However, when injecting volumes into the nucleus pulposus, it is desirable that the volume of drug delivered be no more than about 1 ml, preferably no more than 0.5 ml, (i.e., a maximum of about 0.5 ml) more preferably between about 0.1 and about 0.3 ml. When injected in these smaller quantities, it is believed the added volume will not cause an appreciable pressure increase in the nucleus pulposus. In one embodiment, the cytokine antagonist (for example, p38 MAP kinase inhibitor) is provided in a concentration in the range of about 5 mg/kg to about 50 mg/kg. For example, the range of about 5 mg/kg to about 50 mg/kg is an appropriate range when JNJ 3026582 (RWJ 67657) is placed into DMSO solution to be solubilized.

Accordingly, in some embodiments, the concentration of the antagonist (e.g., a TNF-α antagonist) in the administered drug is at least about 100 mg/ml. When 100 mg of the cytokine antagonist needed to produce the desired therapeutic result, no more than about 1 ml of the drug need be injected. Preferably, the concentration of the cytokine antagonist (e.g., TNF-α antagonist) in the administered drug is at least 200 mg/ml. In this condition, no more than about 0.5 ml of the drug need be injected. Preferably, the concentration of the TNF-α antagonist in the administered drug is at least 500 mg/ml. In this condition, between about 0.03 ml and about 0.3 ml of the drug need be injected. In some embodiments, the concentration of the antagonist (e.g., p38 MAP kinase inhibitor) in the administered drug is at least 100 nanograms/ml.

In some embodiments, the cytokine antagonist is provided in a sustained release (delivery) device. The sustained release device is adapted to remain within the disc for a prolonged period and slowly release the cytokine antagonist contained therein to the surrounding environment. This mode of delivery allows an cytokine antagonist to remain in therapeutically effective amounts within the disc for a prolonged period.

In some embodiments, the cytokine antagonist is predominantly released from the sustained delivery device by its diffusion through the sustained delivery device (preferably, though a polymer). In others, the cytokine antagonist is predominantly released from the sustained delivery device by the biodegradation of the sustained delivery device (preferably, biodegradation of a polymer).

Preferably, the sustained release device (i.e., sustained delivery device) comprises a bioresorbable material whose gradual erosion causes the gradual release of the cytokine antagonist to the disc environment. In some embodiments, the sustained release device comprises a bioresorbable polymer. Preferably, the bioresorbable polymer has a half-life of at least one month, more preferably at least two months, more preferably at least 6 months.

In some embodiments, the sustained release device provides controlled release. In others, it provides continuous release. In others, it provides intermittent release. In others, the sustained release device comprises a biosensor.

In some embodiments, the sustained delivery device comprises a plurality of bioerodable macrospheres. The cytokine antagonist is preferably contained in a gelatin (or water or other solvent) within the capsule, and is released to the disc environment when the outer shell has been eroded. The device can include a plurality of capsules having outer shells of varying thickness, so that the sequential breakdown of the outer shells provides periodic release of the cytokine antagonist.

In some embodiments, the sustained delivery device comprises an inflammatory-responsive delivery system, such as one comprising bioerodable microspheres that are eroded by invading macrophages. This technology provides a high correspondence between physiologic inflammation of disc environment and the release of the cytokine antagonists into that environment. Preferably, the technology disclosed in Brown et al., Arthritis. Rheum., 41(12): 2185-95 (December 1998) is selected.

In some embodiments, the sustained delivery device comprises the devices disclosed in U.S. Pat. No. 5,728,396 (“Peery”), the specification of which is incorporated by reference in its entirety.

In some embodiments, the sustained delivery device comprises a plurality (e.g., at least one hundred) of water-containing chambers, each chamber containing the cytokine antagonist. Each chamber is defined by bilayer lipid membranes comprising synthetic duplicates of naturally occurring lipids. The release of the drug can be controlled by varying at least one of the aqueous excipients, the lipid components, and the manufacturing parameters. Preferably, the formulation comprises no more than 10% lipid. In some embodiments, the DEPOFOAM™ technology of Skyepharma PLC (London, United Kingdom) is selected.

In some embodiments, the sustained delivery device comprises a delivery system disclosed in U.S. Pat. No. 5,270,300 (“Hunziker”), the specification of which is incorporated by reference in its entirety.

In some embodiments, the sustained delivery device comprises the co-polymer poly-DL-lactide-co-glycolide (PLG). Preferably, the formulation is manufactured by combining the cytokine antagonist, the co-polymer and a solvent to form a droplet, and then evaporating the solvent to form a microsphere. The plurality of microspheres are then combined in a biocompatible diluent. Preferably, the cytokine antagonist is released from the co-polymer by its diffusion through and by the biodegradation of the co-polymer. In some embodiments hereof, the PROLEASE™ technology of Alkermes (Cambridge, Mass.) is selected.

In some embodiments, the sustained delivery device comprises a hydrogel. Hydrogels can also be used to deliver the cytokine antagonist in a time-release manner to the disc environment. A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the cytokine antagonist at the application site, thereby eliminating undesired migration from the disc. The hydrogels are also biocompatible, e.g., not toxic, to cells suspended in the hydrogel.

A “hydrogel-cytokine antagonist composition” is a suspension of a hydrogel containing desired cytokine antagonist. The hydrogel-cytokine antagonist composition forms a uniform distribution of cytokine antagonist with a well-defined and precisely controllable density. Moreover, the hydrogel can support very large densities of cytokine antagonist. In addition, the hydrogel allows diffusion of nutrients and waste products to, and away from, the cytokine antagonist, which promotes tissue growth.

Hydrogels suitable for use in the present invention include water-containing gels, i.e., polymers characterized by hydrophilicity and insolubility in water. See, for instance, “Hydrogels”, pages 458-459, in Concise Encyclopedia of Polymer Science and Engineering, Eds. Mark et al., Wiley and Sons, (1990), the disclosure of which is incorporated herein entirely by reference. Although their use is optional in the present invention, the inclusion of hydrogels can be highly advantageous since they tend to contribute a number of desirable qualities. By virtue of their hydrophilic, water-containing nature, hydrogels can:

a) house viable cells, such as mesenchymal stem cells, and

b) assist with load bearing capabilities of the disc.

In a preferred embodiment, the hydrogel is a fine, powdery synthetic hydrogel. Suitable hydrogels exhibit an optimal combination of such properties as compatibility with the matrix polymer of choice, and biocompatability. The hydrogel can include one or more of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

In some embodiments, the sustained delivery device includes a polymer selected from the group consisting of PLA, PGA, PCL and mixtures thereof.

If the half-life of the administered agent within the disc is relatively long, then it may be assumed that a relatively small dose of the administered agent can be administered into the disc. In this condition, the slow depletion of the cytokine antagonist would not cause the administered agent to fall below therapeutically effective levels in the disc until an extended period of time has elapsed.

In some embodiments in which administered agent have long half-lives within the disc, the dose administered can be very small.

For example, if it is believed that a cytokine antagonist is effective when present in the range of about 1-10 mg/kg or 1-10 ppm (as is believed to be the case for the TNF antagonist REMICADE® infliximab), and since a typical nucleus pulposus of a disc has a volume of about 3 ml (or 3 cc, or 3 g), then only about 3-30 μg of the cytokine antagonist need be administered to the disc in order to provide a long lasting effective amount of the drug. As a point of reference, Tobinick discloses that at least 1 mg of a administered agent should be administered perispinally in order to cure back pain. Similarly, Olmarker mixed 100 ml of a formulation comprising 1.11 mg/ml of a monoclonal antibody into 40 mg of an extracted nucleus pulposus, thereby producing a monoclonal antibody concentration of about 3 parts per thousand. The smaller amounts available by this route reduce the chances of deleterious side effects of the cytokine antagonist. In one embodiment, the cytokine antagonist is effective when present in the range of about 5 mg/kg to about 50 mg/kg.

For example, suppose a clinician administered 0.3 ml of 60 mg/ml REMICADE® infliximab into a 2.7 cc disc, thereby producing an infliximab concentration in the disc of about 6 mg/ml, or 6 parts per thousand. Without wishing to be tied to a theory, if infliximab has the same half-life within a nucleus pulposus as it does when administered systemically (i.e., about 1 week), then the concentration of infliximab would remain above about 10 ppm for about 9 weeks. Therefore, if another dose were needed, the clinician would only need to provide the second dose after about two months.

In one embodiment, the administered agent (e.g., cytokine antagonist) is provided in a dose of about 5 μg to about 100 μg. In one embodiment, the cytokine antagonist is provided in a concentration of about 25 μg to about 50 μg.

Therefore, in some embodiments, the administered agent (e.g., cytokine antagonist) is provided in a dose of less than about 1 mg, for example, a maximum of about 0.5 mg, e.g., less than about 0.5 mg, more preferably, less than about 0.1 mg, more preferably less than about 0.01 mg, e.g., less than about 0.001 mg. The smaller amounts available by this route reduce the chances of deleterious side effects of the administered agent (e.g., cytokine antagonist). Preferably, the administered agent (e.g., cytokine antagonist) provided in these smaller amounts is a p38 MAP kinase inhibitor. Preferably, the formulation is administered in an amount effective to reduce pain.

In one embodiment, for local delivery, for example local delivery with a hydrogel or local delivery device, the amount of compound or agent in the tissue can be 0.02 to 50 μM (micro-molar) or per ml of fluid in the cavity to be injected.

In one embodiment, for in vitro assays, the dose can be 5-100 nM (nano-molar). In some embodiments, the dose can be about 0.02-50 μM (micromolar).

In one embodiment, for an in vivo dose, the dose is about 1 cc. In one embodiment, the p38 MAP kinase inhibitor is present in the formulation in an amount of at least about 1 microgram/ml to about 5 milligram/ml, for example, about 5 mg/ml. In some embodiments, the range is about 1 μg/ml-1.2 mg/ml.

In some embodiments, the formulation is administered in an effective amount. In some embodiments, the formulation is administered in an amount effective to reduce pain. In some embodiments, the formulation is administered in an amount effective to inhibit degradation of the ECM of the nucleus pulposus.

In preferred embodiments, the formulation of the present invention is administered directly into the disc through the outer wall of the annulus fibrosus. In one embodiment, the direct administration includes depositing the administered agent (e.g., cytokine antagonist) in the nucleus pulposus portion of the disc. In this condition, the fibrous nature of the annulus fibrosus that surrounds the nucleus pulposus will help keep the cytokine antagonist contained within the disc.

Preferably, the formulation of the present invention is injected into the disc through a small bore needle. More preferably, the needle has a bore of about 22 gauge or less, so that the possibilities of producing a herniation are mitigated. For example, the needle can have a bore of about 24 gauge or less, so that the possibilities of producing a herniation are even further mitigated.

If the volume of the direct injection of the formulation is sufficiently high so as to cause a concern of overpressurizing the nucleus pulposus, then it is preferred that at least a portion of the nucleus pulposus be removed prior to direct injection. In some embodiments, the volume of removed nucleus pulposus is substantially similar to the volume of the formulation to be injected. For example, the volume of removed nucleus pulposus can be within about 80-120% of the volume of the formulation to be injected. In addition, this procedure has the added benefit of at least partially removing some degenerated disc from the patient.

In other embodiments, the formulation is delivered into the disc space through the endplate of an opposing vertebral body. This avenue eliminates the need to puncture the annulus fibrosus, and so eliminates the possibility of herniation.

Although the agent may therapeutically treat the disc by binding the target cytokine, thereby reducing pain and arresting degradation of the ECM, it is believed that at least some of these antagonists do not help repair the damage done by the cytokine to the ECM.

Therefore, there may be a need to provide a therapy that also helps repair the ECM.

In accordance with the present invention, there is provided a method of treating degenerative disc disease in an intervertebral disc having a nucleus pulposus, comprising:
a) administering a cytokine antagonist (e.g., a p38 MAP kinase inhibitor) into a degenerating disc; and
b) administering at least one additional therapeutic agent in an amount effective to at least partially repair the disc.

### Therapeutic Agents

In accordance with one aspect of the invention, both the cytokine antagonist(s) and additional (e.g., second) therapeutic agent(s) are locally administered into the disc. When the cytokine antagonist is specific, it does not interfere with the locally administered additional therapeutic agent, and so each agent may independently work to provide therapy to the diseased disc.

More than one additional therapeutic agent can be administered. For example, there can be third, fourth, fifth or more therapeutic agents.

In some embodiments, the cytokine antagonist and additional therapeutic agent(s) are administered simultaneously. In others, the cytokine antagonist is administered first. In still others, the additional therapeutic agent(s) is/are administered first.

Also included in the scope of the present invention are methods of treating or preventing disc degeneration (e.g., DDD) comprising the administration of one or more of the therapeutic agents described herein.

Other compounds which may be added to the disc include, but are not limited to: any agent recited herein; vitamins and other nutritional supplements; hormones; glycoproteins; fibronectin; peptides and proteins; carbohydrates (both simple and/or complex); proteoglycans; oligonucleotides (sense and/or antisense DNA and/or RNA); bone morphogenic proteins (BMPs); antibodies (for example, to infectious agents, tumors, drugs or hormones); gene therapy reagents; anti-cancer agents; anti-proliferative compounds (agents) (for example, rapamycin and JNJ 7706621); additional cytokine antagonists (for example, TNFα inhibitors such as REMICADE®, IL-6 inhibitors and Il-1β inhibitors); (MMP inhibitors, e.g., batimastat) (BB94, British Biotech Pharmaceuticals, Ltd.); non-steroidal anti-inflammatory drugs (NSAIDS) such as TOLMETIN™, TEPOXALIN™, SUPROL™, diacerein and rhein; anti-inflammatory agents such as centella (ETCA), madecassoside; feverfew; ORC (interceed); suprofen; and tiaprofenic acid. Genetically altered cells and/or other cells may also be included in the matrix of this invention. If desired, substances such as analgesics (pain relievers) and narcotics may also be admixed with a polymer for delivery and release to the disc space.

In one embodiment, the anti-proliferative agent is selected from the group consisting of rapamycin and JNJ 7706621. JNJ 7706621 is represented by the structure in FIG. 13B.

In another embodiment, the agent is selected from the group consisting of tolmetin, tepoxalin, suprofen, tiaprofenic acid; centella (ETCA), madecassoside, rhein, diacerein, feverfew, batimastat and ORC (Interceed).

In one embodiment, healthy cells are introduced into the disc that have the capability of at least partially repairing any damage done to the disc during the degenerative process. In some embodiments, these cells are introduced into the nucleus pulposus and ultimately produce new extracellular matrix for the nucleus pulposus. In others, these cells are introduced into the annulus fibrosus and produce new extracellular matrix for the annulus fibrosus.

In some embodiments, these cells are obtained from another human individual (allograft), while in others embodiments, the cells are obtained from the same individual (autograft). In some embodiments, the cells are taken from an intervertebral disc (and can be either nucleus pulposus cells or annulus fibrosus cells), while in others, the cells are taken from a non-disc tissue (and may be mesenchymal stem cells). In others, autograft chondrocytes (such as from the knee, hip, shoulder, finger or ear) may be used.

Preferably, when viable cells are selected as an additional therapeutic agent or substance, the viable cells comprise mesenchymal stem cells (MSCs). MSCs provide a special advantage for administration into a degenerating disc because it is believed that they can more readily survive the relatively harsh environment present in the degenerating disc; that they have a desirable level of plasticity; and that they have the ability to proliferate and differentiate into the desired cells.

In some embodiments, the mesenchymal stem cells are obtained from bone marrow, preferably autologous bone marrow. In others, the mesenchymal stem cells are obtained from adipose tissue, preferably autologous adipose tissue.

In some embodiments, the mesenchymal stem cells injected into the disc are provided in an unconcentrated form. In others, they are provided in a concentrated form. When provided in concentrated form, they can be uncultured. Uncultured, concentrated MSCs can be readily obtained by centrifugation, filtration, or immuno-absorption. When filtration is selected, the methods disclosed in U.S. Pat. No. 6,049,026 (“Muschler”), the specification of which is incorporated by reference in its entirety, are preferably used. In some embodiments, the matrix used to filter and concentrate the MSCs is also administered into the nucleus pulposus. If this matrix has suitable mechanical properties, it can be used to restore the height of the disc space that was lost during the degradation process.

In some embodiments, growth factors are additional therapeutic agents. As used herein, the term “growth factors” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and FGF-2) and FGF-4; members of the platelet-derived growth factor (PDGF) family, including PDGF-AB, PDGF-BB and PDGF-AA; EGFs; members of the insulin-like growth factor (IGF) family, including IGF-I and -II; the TGF-β superfamily, including TGF-β1, 2 and 3 (including MP-52); osteoid-inducing factor (OIF), angiogenin(s); endothelins; hepatocyte growth factor and keratinocyte growth factor; members of the bone morphogenetic proteins (BMP's) BMP-1, BMP-3; BMP-2; OP-1; BMP-2A, BMP-2B, and BMP-7, BMP-14; HBGF-1 and HBGF-2; growth differentiation factors (GDF's), members of the hedgehog family of proteins, including indian, sonic and desert hedgehog; ADMP-1; members of the interleukin (IL) family, including IL-1 thru IL-6; GDF-5 and members of the colony-stimulating factor (CSF) family, including CSF-1, G-CSF, and GM-CSF; and isoforms thereof.

In some embodiments, the growth factor is selected from the group consisting of TGF-β, bFGF, and IGF-1. These growth factors are believed to promote regeneration of the nucleus pulposus. Preferably, the growth factor is TGF-β. More preferably, TGF-β is administered in an amount of between about 10 ng/ml and about 5000 ng/ml, more preferably between about 50 ng/ml and about 500 ng/ml, more preferably between about 100 ng/ml and about 300 ng/ml.

In some embodiments, the growth factor is a growth differentiation factor. In some embodiments, the growth factor is MP-52. In some embodiments, the growth factor is GDF-5. The protein encoded by this gene is a member of the bone morphogenetic protein (BMP) family and the TGF-beta superfamily. This group of proteins is characterized by a polybasic proteolytic processing site which is cleaved to produce a mature protein containing seven conserved cysteine residues. The members of this family are regulators of cell growth and differentiation in both embryonic and adult tissues.

In some embodiments, platelet concentrate is provided as an additional therapeutic agent. In one embodiment, the growth factors released by the platelets are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it contains growth factors that can restimulate the growth of the ECM, and because its fibrin matrix provides a suitable scaffold for new tissue growth.

In addition, cycline compounds may also be used as an additional therapeutic agent in accordance with the present invention. Preferably, the cycline compound is administered in an amount effective to inhibit the action of a pro-inflammatory cytokine (such as TNF-α) or MMP. Preferably, the cycline compound is administered in an amount effective to inhibit the action of an MMP released by cells during the degenerative process. More preferably, the cycline compound is administered in an amount effective to both a) inhibit the action of a specific pro-inflammatory cytokine (such as TNF-α), and b) inhibit the action of an ECM-degrading MMP released by cells during the degenerative process.

In some embodiments, the cycline compound is selected from the group of cycline compounds consisting of doxycycline, lymecycline, oxicycline compound, tetracycline, minocycline, chemically modified cycline compound (CMT) and KB-R7785. Preferably, doxycycline is selected.

In some embodiments, anti-inflammatory agents such as an antagonist of PPAR-α, are selected.

Since it is known that many pro-inflammatory molecules play a role in joint degeneration, and that the antagonists of the present invention are highly specific, it is further believed that injecting at least two of the highly specific antagonists of the present invention directly into the joint space would be advantageous.

In addition, non-steroidal anti-inflammatory drugs (NSAIDs) may also be selected as additional agents (e.g., second therapeutic agent). In some embodiments, the NSAID is anabolic, and is preferably selected from the group consisting of TOLMETIN™ (available from Ortho-MacNeil), SUPROL™ (available from Johnson & Johnson), and Tiaprofenic acid, (available from Roussel Labs). Preferably, the anabolic NSAID is administered in a dosage sufficient to produce an initial local tissue concentration of between about 5 ug/kg and about 500 ug/kg. In some embodiments, the NSAID is a dual inhibitor of both the COX and LOX pathways, and is preferably TEPOXALIN™ (available from Johnson & Johnson).

In addition, anti-cathepsins may also be used in accordance with the present invention. It is believed that inhibition of these enzymes inhibits the breakdown of the extracellular matrix. Preferably, the antagonists inhibits a cathepsin selected from the group consisting of cathepsin B, cathepsin L and cathepsin K.

In accordance with the present invention, there is provided a method of treating degenerative joint disease, comprising trans-capsularly administering a formulation comprising a p38 MAP kinase inhibitor and at least two additional therapeutic agents selected from the group consisting of:
i) an inhibitor of a pro-inflammatory interleukin;
ii) an inhibitor of TNF-α synthesis; iii) an inhibitor of membrane-bound TNF-α;
iv) an inhibitor of a natural receptor of TNF-α;
v) an inhibitor of NO synthase;
vi) an inhibitor of PLA₂ enzyme;
vii) an anti-proliferative agent;
viii) an anti-oxidant;
ix) an apoptosis inhibitor selected from the group consisting of EPO mimetic peptides, EPO mimetibodies, IGF-I, IGF-II, and caspase inhibitors; and
x) an inhibitor of MMPs.

Preferably, at least one of the substances is an antagonist of TNF-α. Preferably, the other substance is an antagonist of an interleukin.

Since it is known that many pro-inflammatory proteins play a role in disc degeneration, and that some of the antagonists of the present invention are highly specific, it is further believed that injecting at least two of the highly specific antagonists of the present invention directly into the disc would be advantageous.

Also in accordance with the present invention, there is provided a method of treating degenerative disc disease in an intervertebral disc having a nucleus pulposus, comprising administering a formulation comprising at least two highly specific (high specificity) antagonists of pro-inflammatory cytokines selected from the group consisting of TNF-α, an interleukin (preferably IL-1 (e.g., IL-1β, IL-6 and IL-8), FAS, an FAS ligand, and IFN-gamma.

In some embodiments, at least one of the substances is an antagonist of p38 MAP kinase. Preferably, the other substance is an antagonist of an interleukin or TNFα.

In some embodiments, the formulation comprises a suitable biocompatible carrier such as saline. In some embodiments, the carrier is selected from the carriers disclosed in U.S. Pat. No. 6,277,969 (“Le”), the specification of which is incorporated by reference in its entirety. In some embodiments, the formulation includes a solvent, preferably selected from the group consisting of DMSO and ethanol.

In some embodiments, the formulation is administered through a drug pump.

Also in accordance with the present invention, there is provided a formulation for treating degenerative disc disease, comprising:
a) a cytokine antagonist (e.g., a p38 MAP kinase inhibitor) or other antagonist or inhibitor described herein, and
b) an additional therapeutic agent selected from the group consisting of:
   i) a growth factor,
   ii) viable cells, and
   iii) plasmid DNA.

In some embodiments of this formulation, the cytokine antagonist is selected from the group consisting of antagonists of TNF and antagonists of an interleukin.

Because the causes of low back pain may be myriad, and because of the significant cost of many of these specialized cytokine antagonists, it would be useful for the clinician to first perform a diagnostic test in order to confirm that the targeted disc in fact possesses high levels of the targeted cytokine prior to providing the injection.

In one embodiment, the diagnostic test comprises a non-invasive diagnostic test comprising using magnetic resonance imaging (MRI).

Preferably, the clinician would first perform a discogram in order to identify which disc or discs are responsible for the patient's low back pain. Next, the clinician would perform an invasive or non-invasive test upon the targeted disc in order to confirm the presence of or quantify the level of the pro-inflammatory cytokine.

In one embodiment, the diagnostic test comprises an invasive test in which a portion of the disc is removed and analyzed. In some embodiments, the clinician removes a portion of the nucleus pulposus. In some embodiments, the clinician removes a portion of the annulus fibrosus. Preferably, the removed material is a portion of the nucleus pulposus. The presence of pro-inflammatory cytokines in the removed material may detected by procedures including, but not limited, to electrophoresis, or an enzyme-linked immunoabsorbent assay (ELISA) (as per Burke, Br. JBJS, 84-B(2), 2002).

In some embodiments, the diagnostic methods disclosed in U.S. Pat. No. 6,277,969 (“Le”), the specification of which is incorporated by reference in its entirety, are selected. In these methods, high specificity anti-cytokine (e.g., anti-TNFα) compounds are used as diagnostic tools for detecting the cytokine in the patient known or suspected to have a high level of the cytokine.

For example, after determining the levels of the different pro-inflammatory cytokines in the degenerating disc, the clinician will preferably proceed to compare these diagnosed levels against pre-determined levels of the pro-inflammatory cytokines. If the diagnosed level of the pro-inflammatory cytokine exceeds the pre-determined level, then the clinician may conclude that these higher levels are causing unwanted inflammatory action and proceed to directly inject a specific cytokine antagonist (e.g., a p38 MAP kinase inhibitor) into the disc capable of inhibiting the targeted protein.

In some embodiments, the predetermined level for an interleukin is at least 100 pg/ml. In some embodiments, the predetermined level for IL-6 is at least 250 pg/ml. In some embodiments, the predetermined level for IL-8 is at least 500 pg/ml. In some embodiments, the predetermined level for PGE₂ is at least 1000 pg/ml. In some embodiments, the predetermined level for TNF-α is at least 500 pg/ml. In others, the predetermined level for TNF-α is at least 20 pg/ml, more preferably at least 30 pg/ml, more preferably at least 50 pg/ml, more preferably at least 1 ng/ml. In others, the predetermined level for TNF-α is at least 1 ng/disc, and in others, it is at least 1000 pg/disc.

It would also be useful to be able to determine whether directly administering the therapeutic substances of the present invention is, in fact, efficacious. Accordingly, one can measure the level of cytokine remaining in the disc after administration.

The present invention can also be used to prevent degeneration of an intervertebral disc in a human individual, namely, by following a procedure comprising the steps of:
a) determining a genetic profile of the individual,
b) comparing the profile of the individual against a pre-determined genetic profile level of at-risk humans,
c) determining that the individual is an at-risk patient, and
d) injecting an antagonist of the pro-inflammatory protein (e.g., p38 MAP kinase) into a disc of the individual.

Transdiscal administration of an effective amount of other antagonists of pro-inflammatory processes can also help provide therapy to the patient having degenerative disc disease (DDD). In many embodiments, the antagonist is a high specificity antagonist of an enzyme, such as a kinase.

It is further believed that transdiscal administration of an effective amount of an antagonist, e.g., an antagonist of the COX-2 enzyme would also help provide therapy to the patient having DDD. It is believed that the COX-2 enzyme is a regulator of the production of prostaglandins, which are involved both in inflammation and the generation of pain.

Therefore, in accordance with another embodiment of the present invention, there is provided a method of treating degenerative disc disease in an intervertebral disc having a nucleus pulposus, comprising transdiscally administering an effective amount of a formulation comprising an antagonist of COX-2 enzyme into an intervertebral disc.

Typical antagonists of the COX-2 enzyme include Celecoxib (Searle), Rofecoxib (Merck), Meloxican (Boehringer Manheim), Nimesulide, diclofenac and Lodine.

It is further believed that transdiscal administration of an effective amount of an antagonist of the NO synthase enzyme would also help provide therapy to the patient having DDD. It is believed that the NO synthase enzyme regulates the production of NO, which is known to have pro-inflammatory effects.

Therefore, in accordance with another embodiment of the present invention, there is provided a method of treating degenerative disc disease in an intervertebral disc having a nucleus pulposus, comprising transdiscally administering an effective amount of a formulation comprising an antagonist of NO synthase into an intervertebral disc.

Examples of antagonists include NO synthase are N-iminoethyl-L-lysine (L-NIL), and N^{G}-monomethyl-L-arginine.

In some embodiments, the antagonists of NO synthase may be administered systemically.

It is further believed that transdiscal administration of an effective amount of an anti-oxidant would also help provide therapy to the patient having DDD. It is believed that oxidants degrade the nucleus pulposus extra-cellular matrix. Typical anti-oxidants include free radical scavengers and superoxide dismutase enzymes.

Therefore, in accordance with another embodiment of the present invention, there is provided a method of treating degenerative disc disease in an intervertebral disc having a nucleus pulposus, comprising transdiscally administering an effective amount of a formulation comprising an antioxidant into an intervertebral disc.

In some embodiments, the antioxidants may be administered systemically.

It is further believed that trandiscal administration of an effective amount of an anti-proliferative agent would also help provide therapy to the patient having DDD. It is believed that antiproliferative agents may have an effect on inflammation by affecting inflamed tissues which would limit the production of inflammatory cytokines. In some embodiments, the anti-proliferative is selected from the group consisting of a) rapamycin; b) an inhibitor of cyclin dependent kinase 9 (CDK); and c) statins (such as MEVASTATFN™ and LOVASTATIN™). In one embodiment, when rapamycin is selected, a dosage producing a local tissue concentration of between about 0.5 ug/kg and 50 ug/kg is used. For local delivery, for example from a gel or device, the amount of drug in tissue can be expressed in molar terms, for example 1 to 50 uM (micro-molar) or per gm of tissue.

Therefore, in accordance with another embodiment of the present invention, there is provided a method of treating degenerative disc disease in an intervertebral disc having a nucleus pulposus, comprising transdiscally administering an effective amount of a formulation comprising an anti-proliferative agent into an intervertebral disc.

In some embodiments, the anti-proliferative agent is rapamycin or JNJ 7706621. Rapamycin is a potent inhibitor of downstream signaling of TOR (target of Rapamycin) proteins. As such, it is responsible for coordinating the balance between protein synthesis and protein degradation. It is believed that DDD is propagated by a loss of balance between extracellular matrix synthesis and degradation. Since TOR proteins regulate multiple metabolic pathways, rapamycin may stabilize the balance of the cycle. Rapamycin may also directly affect the proliferation and subsequent immune reaction of chondrocytes. In addition, degenerative chondrocytes demonstrate a low level of proliferative activity in contrast to normal chondrocytes which show no activity. This is thought to lead to chondrocyte clustering within the cartilage. Rapamycin could function to eliminate the atypical chondrocyte proliferation. In one embodiment, it is provided in an about 0.1 to about 10 μM dose. In some embodiments, the anti-proliferative agent is rapamycin and JNJ 7706621.

CDK inhibitors may directly affect the proliferation and subsequent immune reaction of chondrocytes. A CDK inhibitor may also have a direct effect on chondrocyte clustering which is known to be a characteristic degenerative event. Exemplary CDK inhibitors include flavopiridol, roscovitine, and compounds disclosed in PCT Patent Publication No. WO 02/057240 (Lin) and U.S. provisional patent application 60/257,703, the specifications of which are incorporated by reference herein in their entirety. In one embodiment, it is provided in a 1 to 10 μM dose. JNJ 7706621 is a CDK inhibitor. Cyclin-dependent kinases (CDKs) are a family of protein serine threonine kinases that play a central role in the molecular machinery that runs the cell cycle.

The present invention is also directed to providing an anti-apoptosis molecule to the diseased disc. These molecules serve to protect against chondrocyte apoptosis. Preferred compounds include EPO, erythropoetin mimetic peptides, EPO mimetibodies, IGF-I, IGF-II, and caspase inhibitors.

Therefore, in accordance with another embodiment of the present invention, there is provided a method of treating degenerative disc disease in an intervertebral disc having a nucleus pulposus, comprising transdiscally administering an effective amount of a formulation comprising a cytokine antagonist and an anti-apoptotic agent into an intervertebral disc.

In some preferred embodiments, the antagonist is combined in the formulation with a viscosupplement. The viscosupplement has characteristics substantially similar to that of natural healthy nucleus pulposus ECM.

Preferably, the viscosupplement comprises glycosaminoglycans (GAGS). GAGS are biopolymers consisting of repeating polysaccharide units, and are present in nature on the cell surface as well as in the extracellular matrix of animals. GAGS are long unbranched polysaccharides containing a repeating disaccharide unit. The disaccharide unit contains either of two modified sugars, N-acetylgalactosamine or N-acetylglucosamine and a uronic acid such as glucuronate or iduronate. GAGS are highly negatively charged molecules, with extended conformation that imparts high viscosity to the solution. In addition, to high viscosity, GAGS routinely possess low compressability, which makes these molecules ideal for a lubricating fluid in the joints. At the same time, their rigidity provides structural integrity to cells and provides passageways between cells, allowing for cell migration.

Hyaluronic acid (HA) is a high molecular weight polysaccharide of N-acetyl glucosamine and glucuronic acid molecules that is naturally occurring in all mammals in a variety of tissue and some bacterial species. For the purposes of this invention, HA includes any derivatives such as hyaluronan and hyaluronic acid itself with H⁺ ion attached to the COO⁻ group, and salts of hyaluronic acid whereby another positive ion replaces the H⁺ ion, as for example, with Na⁺ which forms sodium hyaluronate. Also included in the definition of HA is any physically or chemically cross-linked hyaluronic acid or derivative. HA is unique among the GAGS in that it does not contain any sulphate and is not found covalently attached to proteins as a proteoglycan. HA polymers are very large, with molecular weights of between about 100,000 and 10,000,000, and can displace a large volume of water. For the purposes of the present invention, a preferred embodiment includes a non-cross linked HA with a molecular weight of 0.5-10 M Dalton.

Preferably, the viscosupplement is selected from the group consisting of hyaluronic acid and hyaluronate (either cross-linked or uncross-linked).

In some embodiments, cartilage cells (which may be from either an allogeneic or autologous source) or mesenchymal stem cells, may be genetically modified to produce a cartilage anabolic agent which could be chosen from the list of growth factors named herein. The production of these chondroprotective agents, differentiation promoting agents would lead to tissue repair.

In one embodiment, the additional therapeutic agent is postpartum-derived cells (PPDCs), which are also known as postpartum cells. The PPDCs can be placenta-derived cells (PDCS) or human Umbilical Tissue-derived Cells (hUTCs). Methods for isolating and collecting PPDCs are described in U.S. application Ser. No. 10/877,446 and 10/877,012, which are incorporated by reference herein in their entirety.

Recent work has shown that plasmid DNA will not elicit an inflammatory response as does the use of viral vectors. Genes encoding cartilage (anabolic) agents such as BMP may be efficacious if injected into the joint. In addition, overexpression of any of the growth factors provided herein or other agents such as TIMP which would limit local MMP activity would have positive effects on chondrocyte and ECM protection. In one embodiment, the plasmid contains the genetic code for human TGF-β or EPO.

Further information regarding the invention may be found in patent application Attorney Docket No. 3518.-008, by Laura J. Brown et al.; Titled: “Trans-Capsular Administration of P38 Kinase Inhibitors Into Orthopedic Joints”, filed December 2007, the contents of which are incorporated by reference in their entirety.

In addition, the following U.S. applications are incorporated herein by reference in their entirety: U.S. Ser. Nos. 10/456,948; 10/610,355; 10/631,487(Pub. No. US 2004/0229878); and 10/630,227.

The teachings of all patents, patent applications and references cited herein are incorporated by reference in their entirety.

### EXAMPLE I

This non-limiting prophetic example describes how to administer transdiscally a formulation comprising a cytokine antagonist, such as a high specificity p38 MAP kinase antagonist, and saline into a nucleus pulposus of a degenerating disc.

First, a clinician uses a diagnostic test to verify that a particular disc within a patient has levels of a particular pro-inflammatory cytokine, such as p38 MAP kinase, in excess of normal levels.

Next, the clinician provides a local anesthetic (such as 5 ml lidocaine) to the region dorsal of the disc of concern to reduce subcutaneous pain.

Next, the clinician punctures the skin of the patient dorsal to the disc of concern with a relatively large (e.g., 18-19 gauge) needle having a stylet therein, and advances the needle through subcutaneous fat and dorsal sacrolumbar ligament and muscles to the outer edge of the intervertebral disc.

Next, the stylet is removed from the needle.

Next, the clinician receives a syringe having a smaller gauge needle adapted to fit within the larger gauge needle. This needle is typically a 22 or 24 gauge needle. The barrel of the syringe contains the formulation of the present invention. In one example, the formulation contains REMICADE® infliximab, and has an infliximab concentration of between about 30 mg/ml and about 60 mg/ml. In another example, the formulation contains a p38 MAP kinase inhibitor at a concentration of about 100 nanograms/ml.

Next, the physician advances the smaller needle co-axially through the larger needle and past the distal end of the larger needle, thereby puncturing the annulus fibrosus. The smaller needle is then further advanced into the center of the nucleus pulposus. Finally, the clinician depresses the plunger of the syringe, thereby injecting between about 0.1 and 1 ml of the formulation into the nucleus pulposus.

### EXAMPLE II

This non-limiting prophetic example is substantially similar to that of Example I, except that the formulation comprises a sustained release device comprising the co-polymer poly-DL-lactide-co-glycolide (PLG). In one example, the formulation contains infliximab as the antagonist, and has an infliximab concentration of between about 30 mg/ml and about 60 mg/ml. In another embodiment, the formulation contains a p38 MAP kinase inhibitor at a concentration of about 100 nanograms/ml.

### EXAMPLE III

### Chondrocyte Pellet Culture Model

Cytokines, such as IL-1β, have significant effects on matrix molecule expression by articular chondrocytes, decreasing type II collagen and aggrecan expression. In addition, such cytokines also have effects on apoptosis, inducible nitric oxide (NO) synthase expression, and matrix metalloproteinase expression. Cytokines also blunt chondrocyte compensatory synthesis pathways required to restore the integrity of the degraded extracellular matrix (ECM). The inventors have demonstrated herein that, in the presence of IL-1β, a chondrocyte pellet undergoes proteoglycan degradation that mimics in vivo the osteoarthritis (OA) condition. This process was fully inhibited by the presence of aggrecanase inhibitors such as batimastat and this model was successfully used for screening aggrecanase inhibitors and other agents.

A chondrocyte pellet culture model was developed for in vitro high throughput screening of therapeutic compounds for inhibition of IL-1β-stimulated matrix degradation. The chondrocyte pellet culture model can be used to detect various potential therapeutic compounds effective in blocking different processes caused by IL-1β stimulation. For example, IL-1β stimulation results in increased glycosaminoglycan (GAG) degradation, prostaglandin E₂ (PGE₂) synthesis and total nitrite/nitrate production. Compounds with different mechanisms of action may inhibit the increase of one or two or all three of these parameters. For this reason, screening by, for example, measuring only GAG release would be limited to detection of a specific class of inhibitors and may not detect compounds with diversified mechanisms of function which may be involved in inhibition of the IL-1 stimulated inflammatory process. The development of the chondrocyte pellet culture assay system in a 96-well plate made possible large-scale screening of potential therapeutic compounds with different mechanisms of action.

In addition, human chondrocytes also release nitric oxide (NO) and prostaglandin E₂ (PGE₂) during OA pathogenesis. Since both NO and PGE₂ have modulating effects on matrix synthesis, cytokine-induced production of both molecules can represent mediators of cartilage degeneration and human knee joints. Therefore, measurement of NO and PGE₂ levels in addition to assessment of GAG release allows for detection of potential therapeutic compounds with different mechanism of actions other than inhibition of proteases directly involved in matrix degradation.

The following in vitro parameters were evaluated using this model:
a) GAG release in the media—a measurement of proteoglycan degradation which indicates cartilage extracellular matrix breakdown,
b) Total Nitric Oxide (NO) production—a measurement of NO production which indicates the presence of inflammatory response or mitogenic stimuli, and
c) PGE₂ levels (by Enzyme-linked Immunosorbent Assay (ELISA))-PGE₂ is a primary product of arachidonic acid metabolism that is synthesized and released upon cell activation, and whose presence indicates an inflammatory response.

### Materials and Methods

### Materials:

Dulbecco's Modified Eagle Medium (DMEM) with high glucose (Cat. No 10564011), Antibiotic-Antimycotic (100×) containing penicillin, streptomycin, amphotericin B, and neomycin (10000 U/ml, 10 μg/ml, 25 mg/ml and 5 μg/ml, respectively) (Cat. No. 15240062), MEM Non-Essential Amino Acids Solution 10 mM (100×), liquid (Cat. No. 11140-050) and phosphate buffered saline (PBS, Cat. No. 14130-144) were purchased from Invitrogen Life Technology. Dimethylethylene (341088), ascorbic acid (A-4403), L-proline (P-5607), tolmetin sodium salt dihydrate (T-6779), diacerein (D9302), rhein (R-7269), indomethacin (1-7378), lipopolysaccharide (L-6529), insulin-transferrin-sodium selenite media supplement (Cat. No. I-1884), trypan blue (T-8154), and chondroitin 6-sulfate sodium salt from shark cartilage (C-4384), were purchased from Sigma-Aldrich (St. Louis, Mo.). Recombinant human interleukin-1β (IL-113, Cat. No: 201-LB), recombinant human tissue necrosis factor α (TNF-α) (Cat. No. 210-TA), recombinant interferon-γ (Cat. No. 285-IF), and recombinant human IL-6 (Cat. No. 1609-CL-025/CF) were purchased from R&D Systems (Minneapolis, Minn.). Nitrate/Nitrite Colorimetric Assay Kit (Cat. No. 780001), and a prostaglandin E2 detection kit (Cat. No. 514131) were purchased from Cayman Chemicals (Ann Arbor, Mich.). A Cytotoxicity Detection Kit (LDH) (Cat. No. 1 644 793) was purchased from Roche (Nutley, N.J.). Fetal calf serum (FCS), Cat. No. SH30070.03, Lot No. ANF19047) was purchased from Hyclone (Logan, Utah). Collagenase (3.4.24.3) and papain (3.4.22.2) were purchased from Worthington Biochemical Corp (Lakewood, N.J.). BD FALCON™ cell strainers 40 μm (Cat. No. 352340) was purchased from Becton, Dickinson and Company (Franklin Lakes, N.J.). Ninety-six deep-well plates (73520-474) were purchased from VWR International, Inc. (West Chester, Pa.). Alginate Recovered Chondrocyte (ARC) tissues were purchased from Articular Engineering Inc., LLC (Northbrook, Ill.).

### Culture Media:

Chondrocyte medium was prepared with DMEM supplemented with 50 μM ascorbic acid, 20 μM L-Proline, 1× non-essential amino acids and 10% (v/v) FCS. The Chondrocyte Stimulation Medium is a defined medium containing DMEM supplemented with 1% FCS, 1× insulin-transferrin-sodium selenite media supplement and Antibiotics-Antimycotics in the presence or absence of IL-1β.

### Chondrocyte Isolation and Pellet Culture and Aliginate Recovered Chondrocytes:

Calf articular cartilage was obtained from the knee joints of eight to twelve month old animals and was processed within twenty-four hours after sacrifice. After removal of muscle and ligament, the cartilage was kept moist using phosphate buffer solution (PBS) to prevent tissue dehydration, and was aseptically peeled off from subchondral bone with a scalpel, and the cartilage pieces were further minced into smaller pieces (˜0.3-0.5×˜0.2-0.5 cm cubes). The cartilage tissue was then washed 3× one hour with PBS containing 10× Antibiotic-Antimycotic followed by a 1×0.5 hour wash in PBS with 1× Antibiotic-Antimycotic. Chondrocytes were then enzymatically isolated from the tissue by incubation for overnight at 37° C. with gentle agitation in 0.2% collagenase in Dulbecco's modified eagle media (DMEM). After removal of tissue debris by filtration through cell strainer and the residual collagenase by 3× wash in PBS, cells were finally resuspended at a density of 2.5×10⁶ cells/ml in chondrocyte medium. A 1 ml aliquot of the cell suspension was dispensed into either a 15 ml Falcon centrifuge tube or a 96 deep-well plate and centrifuged at 2000×g for five minutes. The chondrocyte pellets were incubated at 37° C. in a humidified atmosphere of 5% CO₂ for two to three weeks with a medium change every other day.

As an alternative to using calf articular cartilage, a commercial Aliginate Recovered Chondrocytes (ARC) system was also used (Articular Engineering, LLC (Northbrook, Ill.)). Use of the commercial system cut down on labor and time in producing chondrocyte pellet. This system produced cartilaginous tissue in vitro using bovine chondrocytes. The first step consisted of culturing chondrocytes in alginate under conditions optimal for the formation of a cell-associated matrix (CM). The second step allowed these cells with their CM, after recovery, to rapidly form and become integrated into a solid mass of cartilage on a porous insert within two weeks.

This tissue is rich in cells surrounded by a cell-associated matrix that is degraded rapidly when the cells are exposed to IL-1. ARC tissues in 3 mm punches at delivery were first dispensed into a 96 deep-well plate, one punch per well, for compound treatment. The subsequent procedures were the same as those used for the chondrocyte pellets.

### Treatment of Pellet Cultures:

Chondrocyte pellets were washed once with 1 ml of Chondrocyte Stimulation Medium and equilibrated in the same medium at room temperature for fifteen to thirty minutes. Compounds were dissolved in H₂O, Ethanol or dimethyl sulfoxide (DMSO) (10⁻² M) according to suppliers' instructions and further diluted with Chondrocyte Stimulation Medium to the required concentrations. DMSO concentrations in the culture media should not exceed 1%; this concentration of DMSO has no effect on cartilage proteoglycan metabolism in response to cytokines.

The pellets were first treated with compounds at desired concentrations at 37° C. for one hour and then incubated for three to five days in the absence or presence of 10 ng/ml of IL-1β. At the end of the incubation, media and pellets were harvested and frozen for further analysis. Pellets were also fixed as needed in 4% (w/v) paraformaldehyde in PBS for histology analysis.

### GAG Degradation Assay:

Glycosaminoglycan (GAG) levels in the culture media were determined by measuring the amount of polyanionic material reacting with 1,9-dimethylmethylene blue (DME) as detected by absorbance at 525 nm, using shark chondroitin sulfate as a standard. The DME blue dye-binding solution was prepared by dissolving 16 mg of DME blue in a solution containing 0.304% glycine, 0.23% sodium chloride (NaCl), 9.5 mM hydrochloric acid (HCl), pH=3.0 with absorbance at 525 equal to 0.31. Proteoglycans and proteoglycan metabolites in chondrocyte pellet were released by digesting the pellet with 125 μg/ml of papain in 0.1 M PBS, pH 6.0, 5 mM cysteine, 5 mM ethylene-diamine-tetra-acetic acid disodium salt (Na₂EDTA) at 50° C. for overnight. Results are reported as either micrograms of GAG per milliliter or percent of total GAG released into culture medium.

### Total Nitrite and Nitrate Assay:

Nitrate/Nitrite levels were measured using the Griess reaction (Green L. C. et al., “Analysis of Nitrate, Nitrite, and [15N]nitrate in Biological Fluids,” Anal. Biochem. 126: 131-138 (1982)) with a Nitrate/Nitrite Colorimetric Assay Kit (Cayman Chemicals), the contents of which are incorporated herein by reference. The assay involved a simple two-step process. The first step involved conversion of nitrate in 50 μl of culture medium to nitrite utilizing nitrate reductase in a reaction volume of 100 μl at room temperature for two hours. The second step involved the addition of 50 μl of both Griess Reagent 1 and 2, which converted nitrite into a deep purple azo compound in ten minutes. Photometric measurement of the absorbance at 540 or 550 nm due to this azo chromophore accurately determined nitrite concentration. This assay kit cannot be used for RPMI (Roswell Park Memorial Institute) derived tissue culture medium due to interference with the colorimetric reaction.

### Prostaglandin E₂ Assay:

Prostaglandin E₂ (PGE₂) was measured with a STAT-Prostaglandin E₂ EIA ELISA kit (Cayman Biochemicals). This assay is based on the competition between PGE₂ from 50 μl samples and a PGE₂-alkaline phosphatase conjugate for a limited amount of PGE₂ monoclonal antibody in a reaction volume of 150 μl at room temperature for one hour. The antibody-PGE₂ complex bound to a goat polyclonal anti-mouse IgG that had been previously attached to the well. After washing to remove any unbound reagents, para-nitrophenyl phosphate (pNPP) was added to the well. The product of this enzymatic reaction absorbs strongly at 412 nm. Because of the large range in response to IL-1β and other cytokines, PGE₂ values were converted to log scale prior to doing t-tests.

### Assessment of Cytotoxicity:

Cell death was assessed by measuring the amount of lactate dehydrogenase (LDH) in the culture supernatant. 10-20 μl of culture medium was incubated with the reaction mixture from the Cytotoxicity Detection Kit (Cat No. 11 644 793 001, Roche). The LDH activity was determined calorimetrically in an enzymatic test based on the conversion of lactic acid to pyruvate in the presence of chromagenic substrate tetrazolium salt INT that was reduced to formazan. The amount of formazan formed was measured at 500 nm, which is proportional to the number of dead cells. Since no purified LDH was available for a standard curve at each measurement, the LDH level for 100% dead cells was measured in each experiment. The lysate for the dead cells was prepared as follows: Chondrocytes were enzymatically isolated as described above and about 0.1 million cells that are equivalent to the number of cells contained in a 3-mm punch of fresh cartilage explant were lysed in 1 ml of PBS with 1% Triton-100. 10-20 μl of the lysate was used in the assay as killed cell control.

All assays were done with triplicate or quadruplicate pellets for each dose.

### Results

### Effects of IL-1β Stimulation on PGE₂ Synthesis, NO Production and GAG Degradation

IL-1β stimulation resulted in increased glycosaminoglycan (GAG) degradation (1.7-4.5 fold increase), prostaglandin E₂ (PGE₂) synthesis (2-200 fold increase) and total nitrite/nitrate production (10-20 fold increase).

### Effects of Diacerein and Rhein on PGE₂ Synthesis, NO Production, GAG Degradation and LDH Toxicity

The chondrocyte pellet culture model was validated with known IL-1 inhibitors (diacerein and rhein (its active metabolite)). Therapeutic levels of diacerein and rhein have been shown to inhibit the synthesis and activity of IL-1β and increase expression of soluble IL-1 receptor, as well as to stimulate anabolic processes. Chondrocyte pellets were prepared as described herein. The pellets were treated for five days with or without diacerein or rhein at indicated concentrations in the presence or absence of 10 ng/ml of IL-1β. Culture media were collected for GAG, PGE₂, NO and lactate dehydrogenase (LDH) measurement.

Both diacerein and rhein inhibited proteoglycan degradation at concentrations as low as 0.054 μM and 0.035 μM respectively (FIG. 1A). Diacerein at 5.4 μM and rhein at 0.35 μM also significantly inhibited NO production (FIG. 1D) yet had no effect on PGE₂ synthesis (FIG. 1C). Diacerein at 5.4 μM or rhein at 3.5 μM had a weaker inhibitory effect for GAG, and rhein at 3.5 μM had a weaker effect for NO than those at their lower concentrations. This might be attributed to their cytotoxicity at higher concentrations, with higher toxicity found for rhein (FIG. 1B). The fact that the mechanisms of action for diacerein or rhein are different from batimastat (an MMP inhibitor that inhibits GAG degradation), yet they also show efficacy in inhibition of GAG degradation and NO production, suggests that these parameters are good measures for therapeutic agents with different mechanisms of action.

### Effects of Antiproliferative Compounds on PGE₂ Synthesis, NO Production and GAG Degradation and LDH Toxicity

To test whether antiproliferative compounds have effects on inhibition of IL-1β stimulated responses in this in vitro model, chondrocyte pellets were treated with either JNJ 7706621 or rapamycin.

Rapamycin is a triene macrolide antibiotic, with anti-fungal, anti-inflammatory, anti-tumor and immunosuppressive properties. It blocks T-cell activation and proliferation, as well as the activation of p70 S6 kinase, and exhibits strong binding to FK-506 binding proteins. Rapamycin also inhibits the activity of the protein mTOR (mammalian target of rapamycin), which functions in a signaling pathway to promote tumor growth.

JNJ 7706621 is an inhibitor for cycline dependent kinase. Cyclin-dependent kinases (CDKs) are a family of protein serine/threonine kinases that play a central role in the molecular machinery that runs the cell cycle.

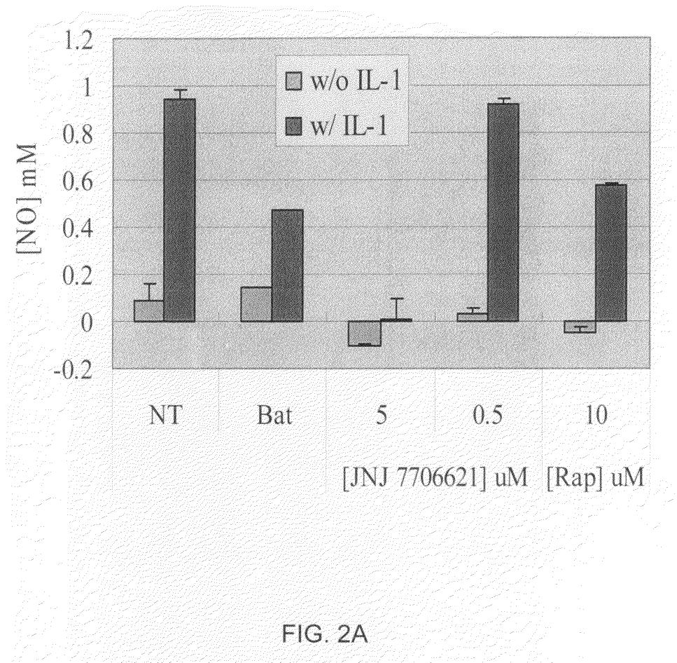

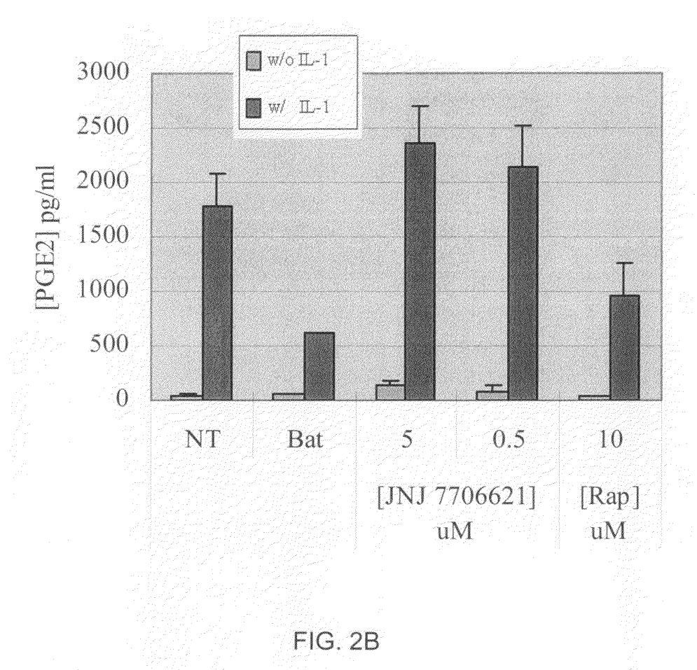

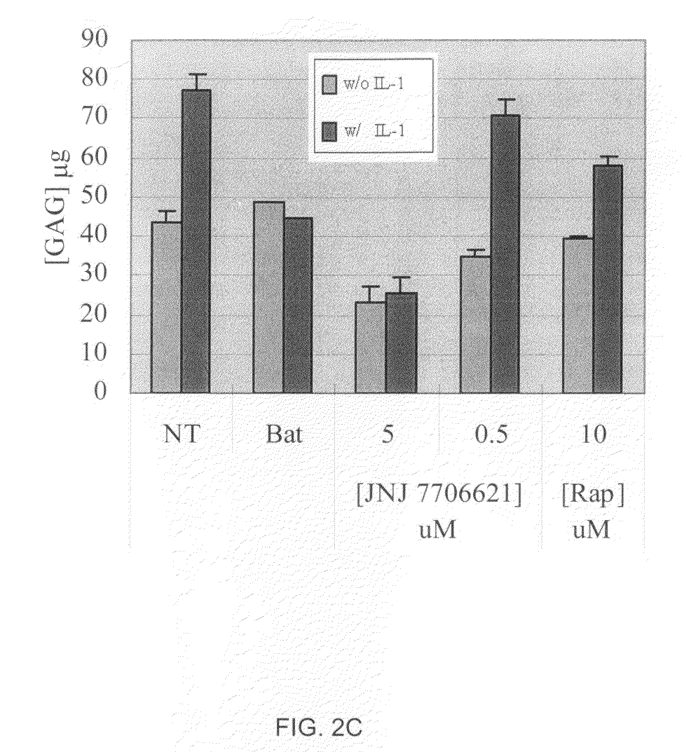

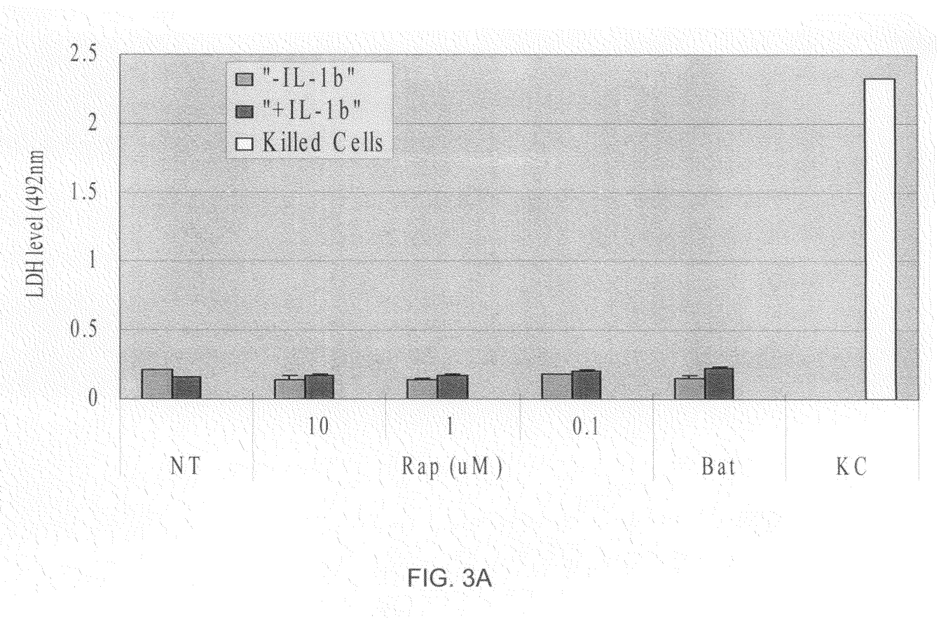

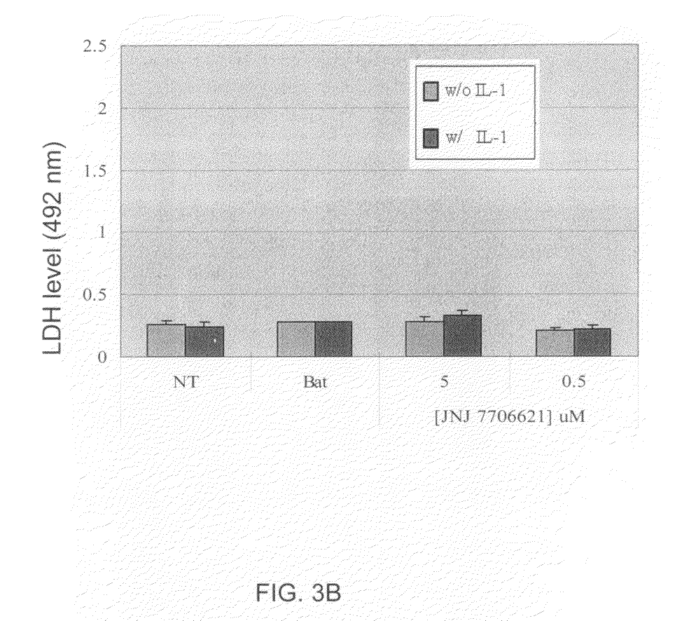

ARC tissues were treated with JNJ 7706621 or rapamycin at the indicated concentrations in the presence or absence of 7.5 ng/ml of IL-1 for five days. Culture media were collected for NO production, PGE₂ synthesis and GAG release according to the methods described in the chondrocyte pellet model. The results show that rapamycin inhibited GAG release and NO production and PGE₂ synthesis. While JNJ 7706621 at 5 μM completely blocked GAG release and NO production, it had little effect on PGE₂ synthesis (FIGS. 2A-C). Both compounds had little toxicity to chondrocytes (FIGS. 3A and 3B).

### Effects of REMICADE® on PGE₂ Synthesis, NO Production and GAG Degradation

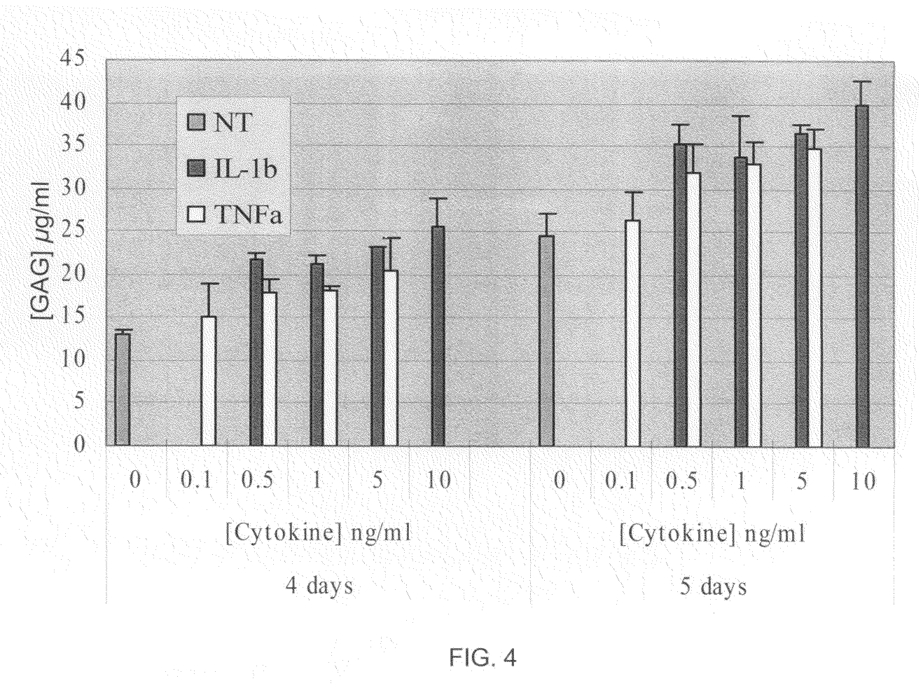

REMICADE® infliximab is a monoclonal antibody against human TNFα. To test whether REMICADE® could modulate cytokine-stimulated responses in chondrocytes, human chondrocyte pellets were first tested for an optimal concentration for cytokine stimulation. Human ARC (i.e., ARCs generated using human chondrocytes) tissues were treated with various concentrations of either TNF or IL-1 for four and five days. The culture media was collected for measurement of GAG content as described above. Other assays were performed as described above. Cytokine dosage at 5 ng/ml and treatment for five days gave a reasonable treatment window, yet was not too challenging for compound efficacy (FIG. 4).

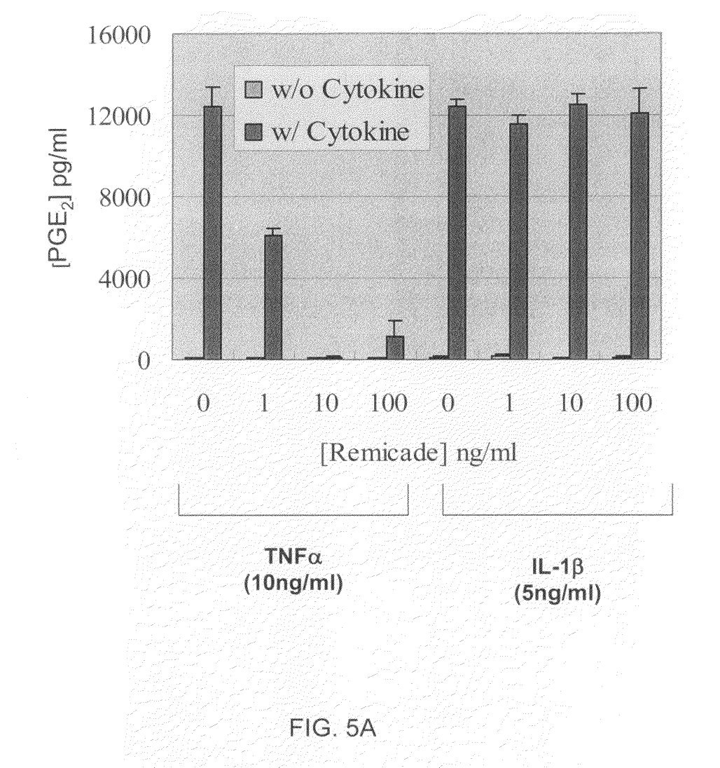

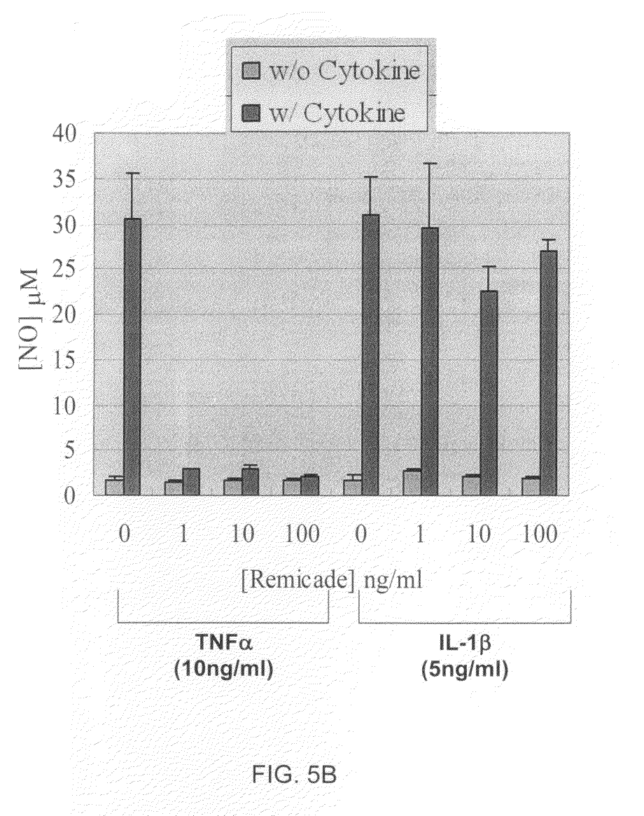

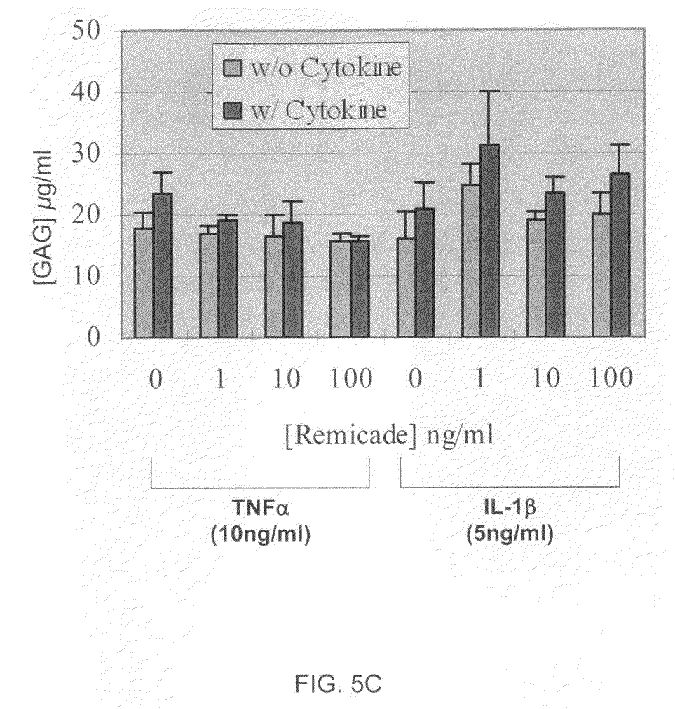

Human ARC tissues were then treated with various concentrations of REMICADE® in the presence or absence of either 10 ng/ml of TNFα or 5 ng/ml of IL-1β for four or five days. While REMICADE® strongly inhibited TNFα-induced PGE₂ synthesis, NO production and GAG release, it had little effect on IL-1β stimulated responses (FIG. 5A-C).

### Effects of a Monoclonal Antibody Against IL-6 on PGE₂ Synthesis, NO Production and GAG Degradation

The efficacy of a monoclonal antibody (mAb) against human IL-6 in inhibition of proteoglycan degradation, NO production and PGE₂ synthesis on human ARC tissues stimulated with IL-6 was also tested. Human ARC tissue were treated with 12.5, 25 and 250 ng/ml of IL-6 mAb diluted into chondrocyte stimulation medium. After one hour of treatment at 37° C., ARCs were stimulated with or without 25 ng/ml IL-6, 250 ng/ml IL-6 soluble receptor (IL-6SR) or IL-6 (25 ng/ml)+L-6SR (250 ng/ml) and incubated for five days before the culture media were harvested for assays.

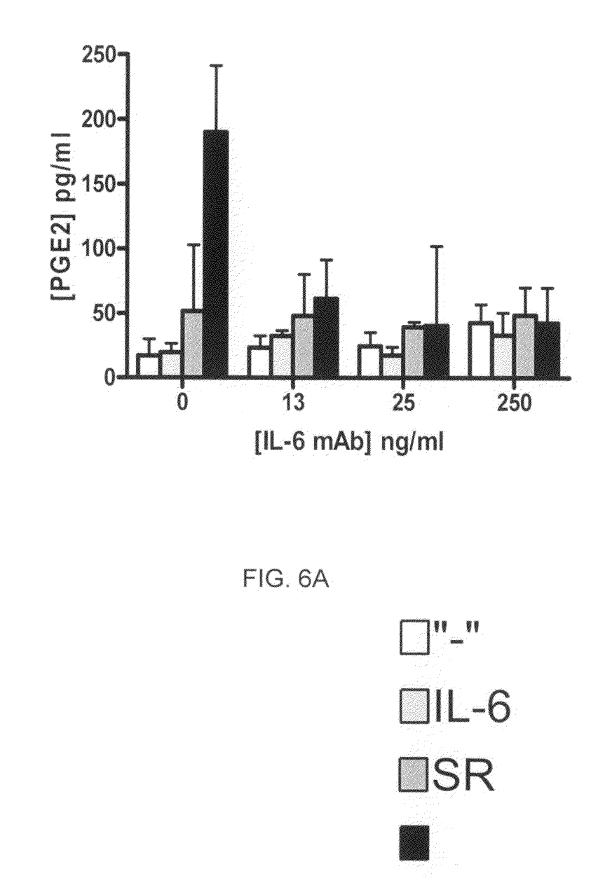

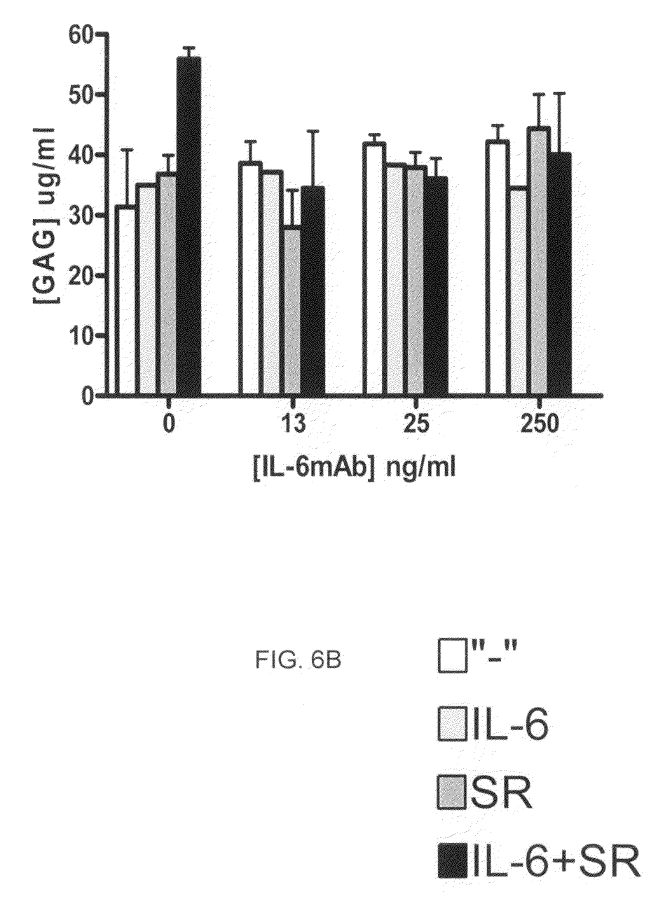

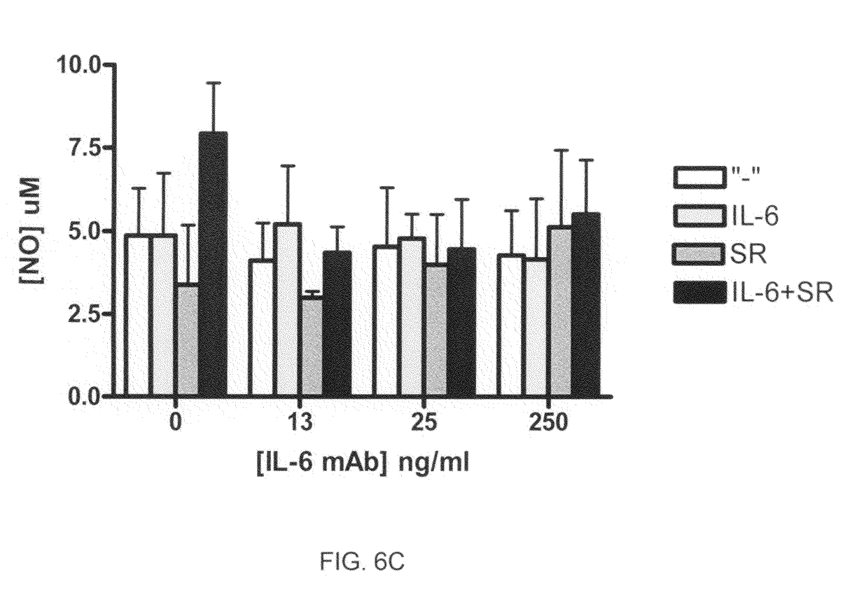

As shown in FIG. 6A-C, neither IL-6 nor IL-6 soluble receptor (IL-6SR) stimulated an inflammatory reaction. However, the combination of IL-6 (25 ng/ml) and IL-6SR (250 ng/ml) at a ratio of 1:10 significantly (t test, P<0.02) stimulated GAG degradation, NO production and PGE₂ synthesis, indicating that the proinflammatory effect of IL-6 requires the presence of L-6SR in this system. This inflammatory effect could be effectively blocked with monoclonal antibody against IL-6 at 12.5 ng/ml.

### Effects of P38 MAP Kinase Inhibitors on PGE₂ Synthesis, NO Production, GAG Degradation and Toxicity

The following p38 MAP kinase inhibitors were assessed for their effect on total nitric oxide (NO) production, PGE₂ levels (by ELISA) and GAG release in the media: JNJ 7583979 (RWJ 351958), JNJ 3026582 (RWJ 67657), JNJ 17089540 (RWJ 669307) SCIO-469 (SD 469) and SCIO-282 (SD 282). After five days of incubation, culture media were collected for measurement of these parameters as discussed above.

Bovine ARC tissues were treated with serial dilutions of JNJ 3026582 (RWJ 67657) (250 pM-10 μM), JNJ 17089540 (RWJ 669307) (25 nM-500 μM) and SCIO-282 (SD 282) (10 pM-10 μM) in the presence of 10 ng/ml of IL-1β or absence of IL-1β. All of these p38 MAP kinase inhibitors significantly inhibited PGE₂ synthesis with IC₅₀˜0.5 μM and NO production with wider ranges of IC₅₀.

As shown in FIG. 7A and FIG. 7B, JNJ 7583979 (RWJ 351958) significantly inhibited PGE₂ synthesis and NO production.

While JNJ 17089540 (RWJ 669307) and SCIO-282 (SD 282) had little effect on inhibition of GAG degradation, JNJ 7583979 (RWJ 351958) and JNJ 3026582 (RWJ 67657) showed significant effect, with stronger efficacy found for JNJ 3026582 (RWJ 67657) (FIG. 8, FIG. 9A, FIG. 9B and FIG. 9C). These results suggest that JNJ 17089540 (RWJ 669307) and SCIO-282 (SD 282) mainly modulate MAP kinase activity that might contribute to aberrant function of downstream transcription factors involved in NO and PGE₂ synthesis and some members also have activities in prevention of matrix degradation.

These compounds showed little cytotoxicity to chondrocytes within the tested concentrations (FIG. 10A, FIG. 10B and FIG. 10C).

Table 2 lists the results from a direct enzyme assay of SCIO-469 (SD 469) and SCIO-282 (SD 282). The assays were performed with human PBMC's stimulated with endotoxin. The information regarding SCIO-469 (SD 469) in comparison with SCIO-282 (SD 282) indicates that, while both compounds have selectivity for p38a, SCIO-282 (SD 282) is ˜6 times more potent than SCIO-469 (SD 469) in inhibition of the enzyme.

TABLE 2
Comparison of Drug Activities on Inhibition of Different
Enzymes SCIO-469 vs. SCIO-282

| | SCIO-469 (SD 469) | SCIO-282 (SD 282) |
|---|---|---|
| h-p38α, IC₅₀ (nM) | 9 | 1.6 |
| h-p38β, IC₅₀ (nM) | 98 | 23 |
| Selectivity, p38β/α | ~10 | ~15 |
| 6 CYP450 (IC₅₀, μM) | >1 | CYP2C9, 0.5 μM |
| TNFα Inhibition | 1.6 | 0.07 |
| h-WBA (EC₅₀, μM) | | (10x diluted) |

h-WBA = human whole blood assay.
EC = effective concentration.

However, in contrast to the results from the direct enzyme assay in Table 2, Table 3A demonstrates that SCIO-469 (SD 469) is more potent than SCIO-282 (SD 282) in inhibition of both NO (18 nM vs 74 nM) and PGE₂ (107 nM vs 583 nM) production in the chondrocyte cell pellet model.

TABLE 3A
Comparison of NO and PGE₂ values SCIO-469 vs. SCIO-282

| | SCIO-469 | JNJ 3026582 | SCIO-282 | JNJ 17089540 |
|---|---|---|---|---|
| IC₅₀ (nM) | (SD 469) | (RWJ 67657) | (SD 282) | (RWJ 669307) |
| NO | 18.01 | 119.3 | 74.39 | 4920 |
| PGE₂ | 106.8 | 421.2 | 582.5 | 29570 |

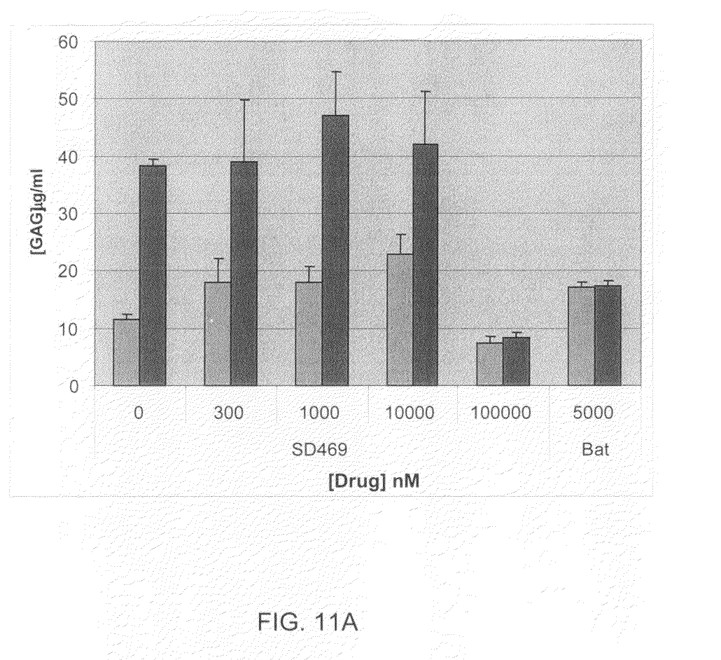

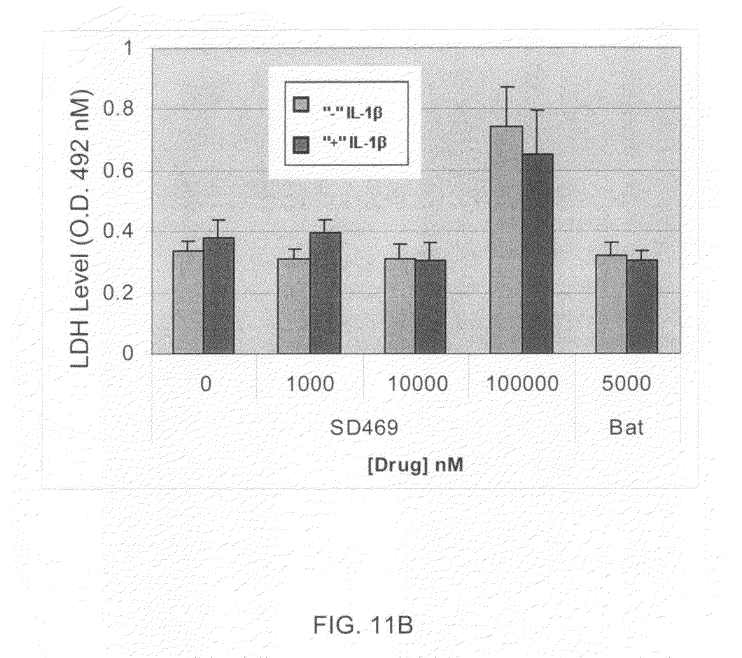

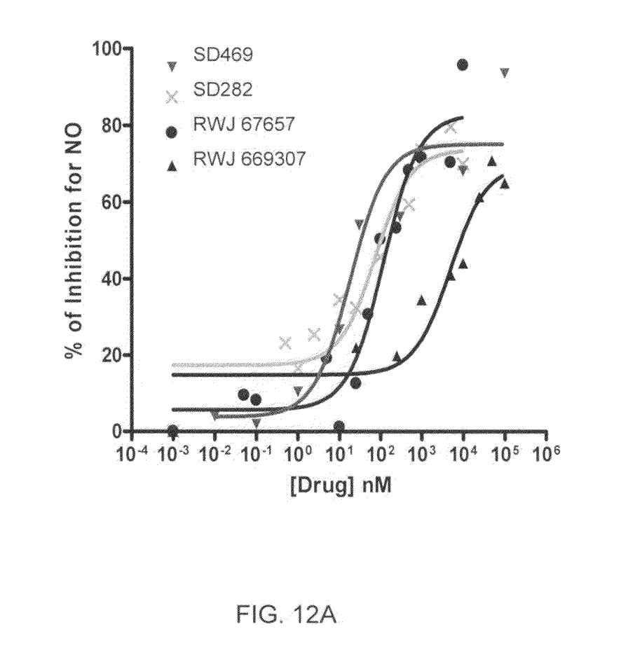

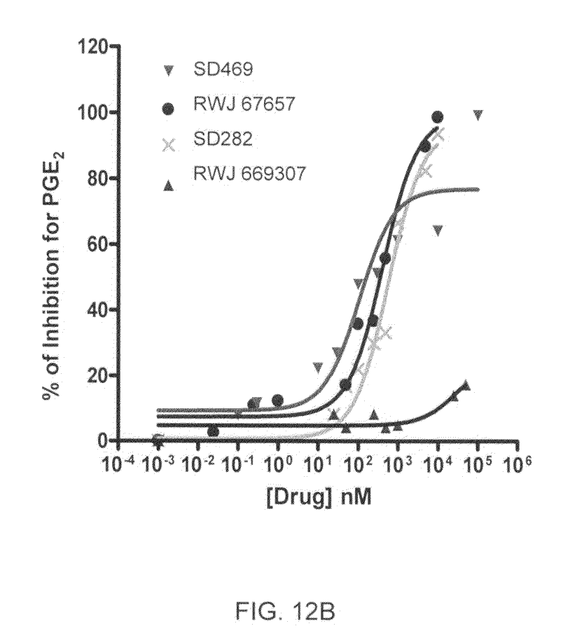

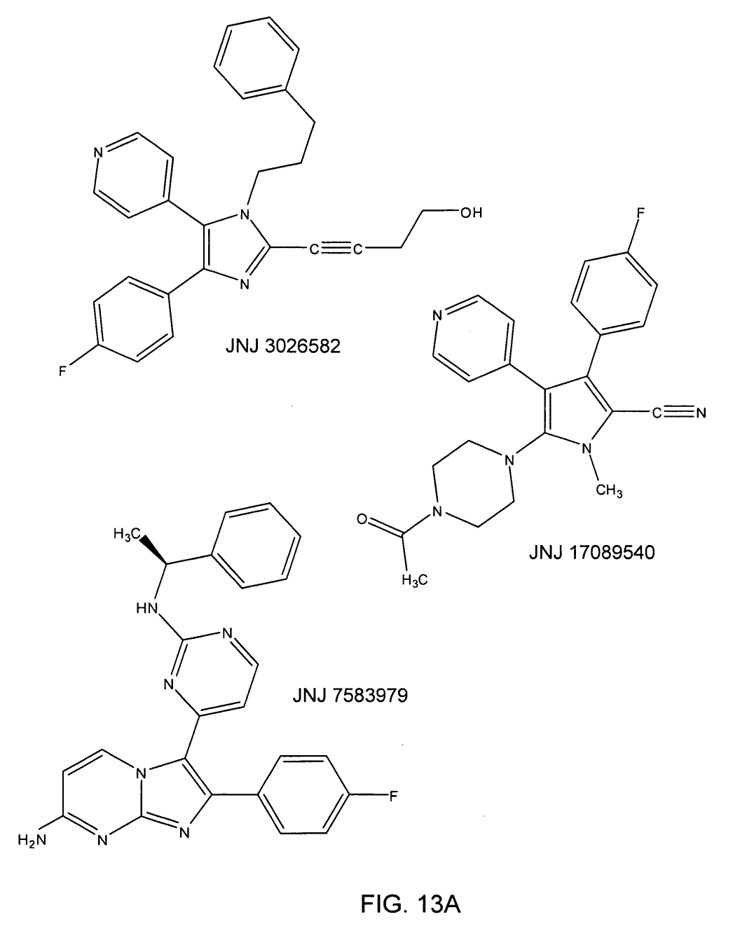

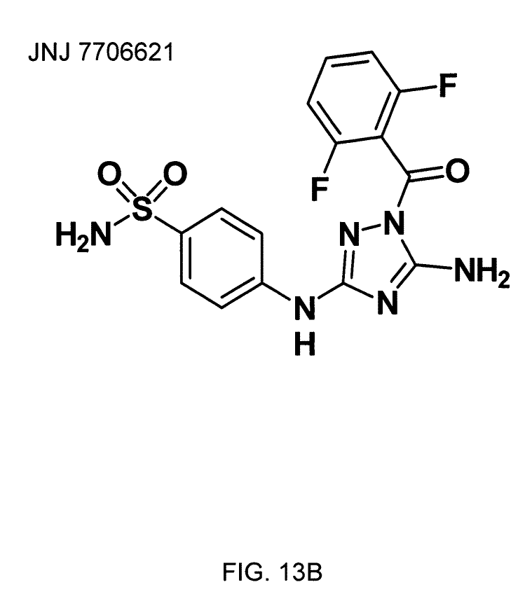

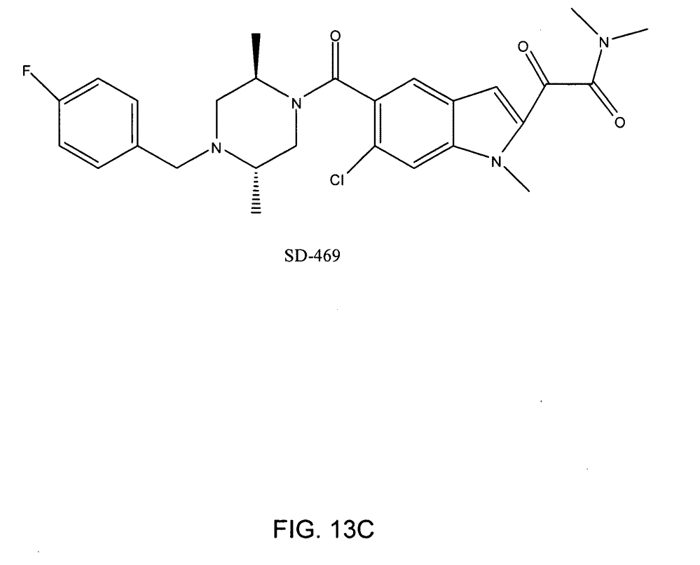

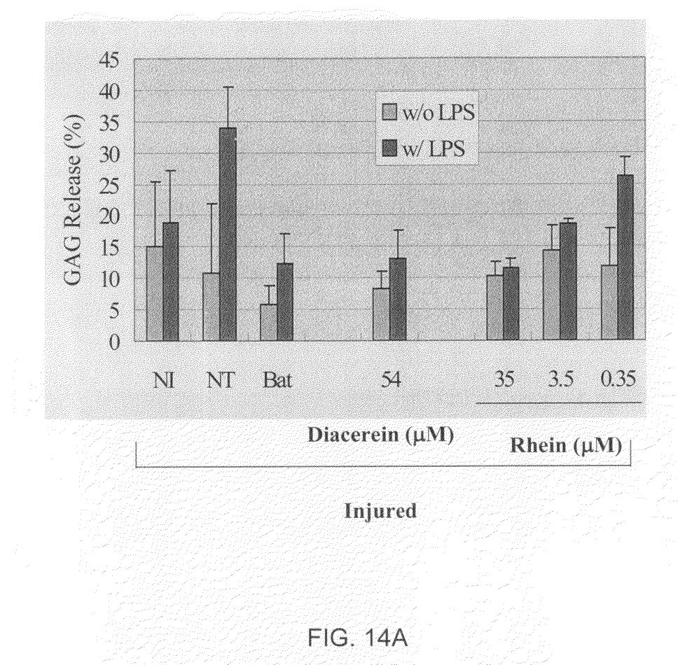

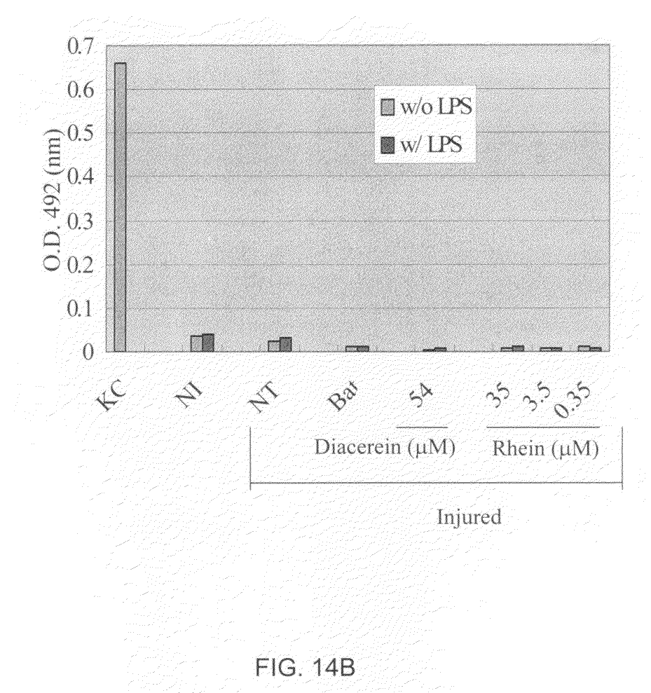

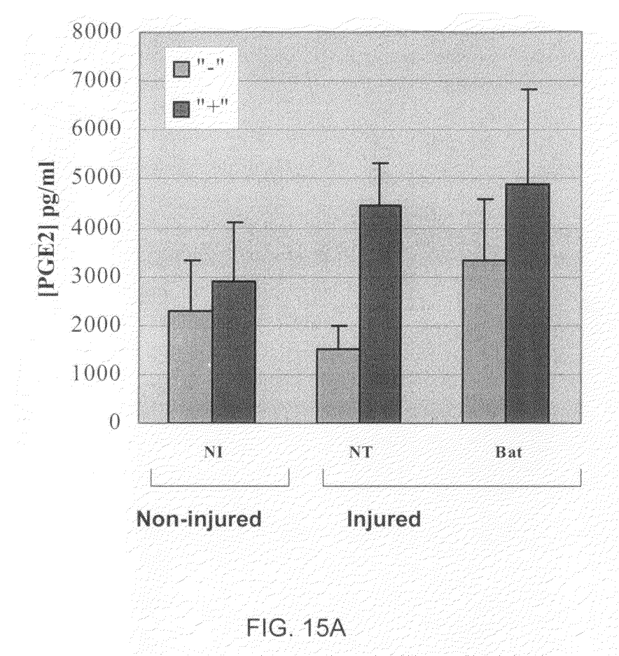

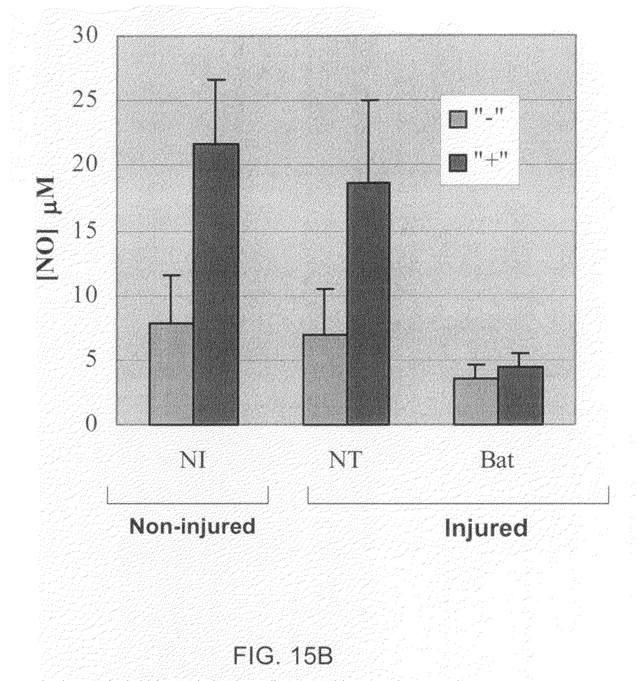

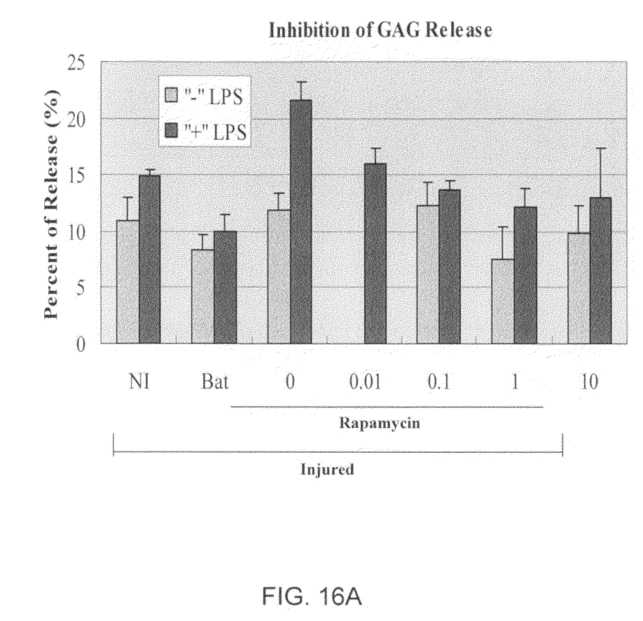

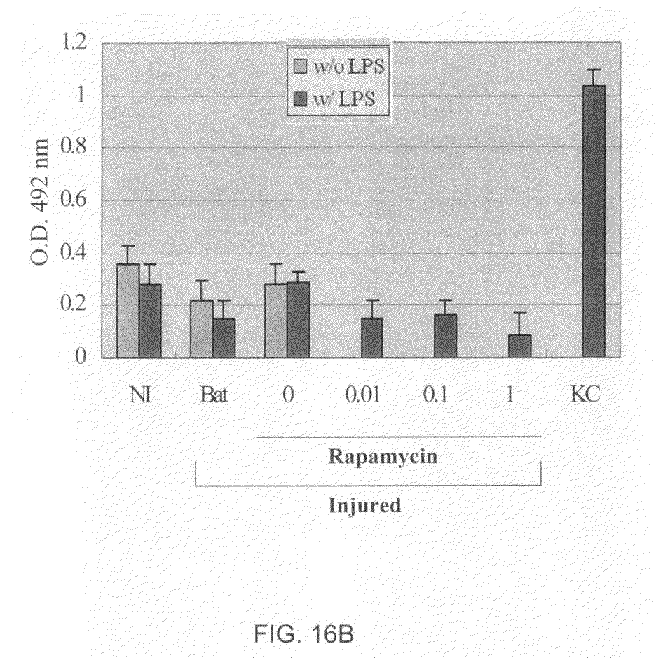

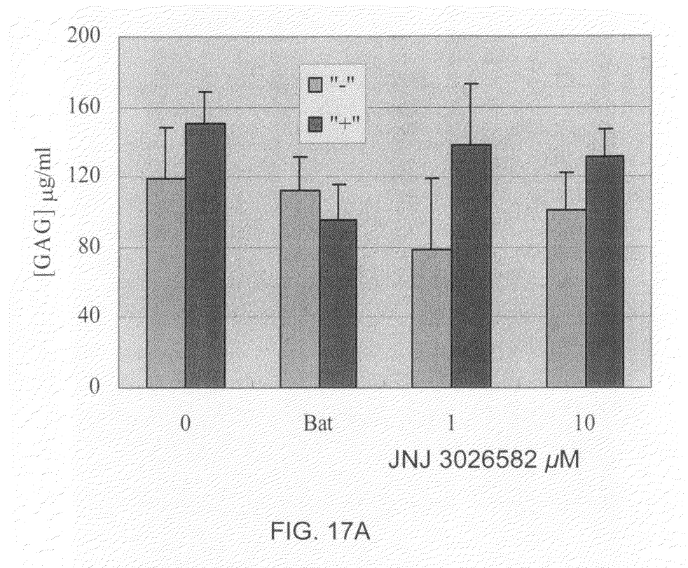

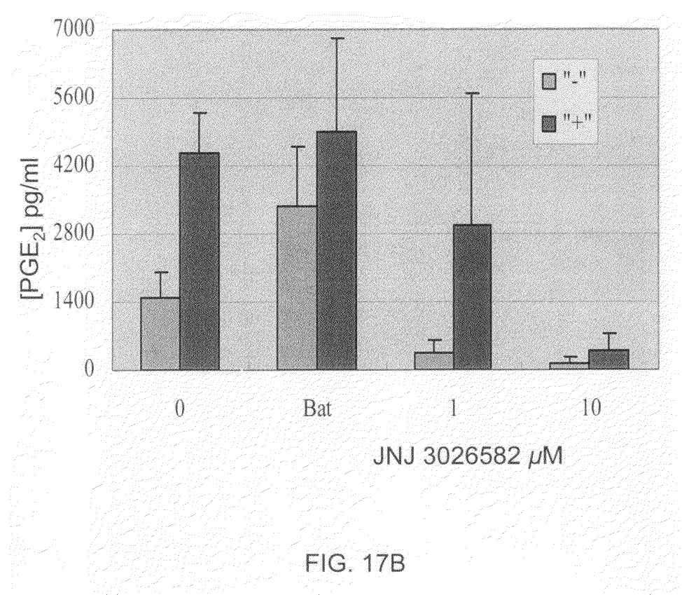

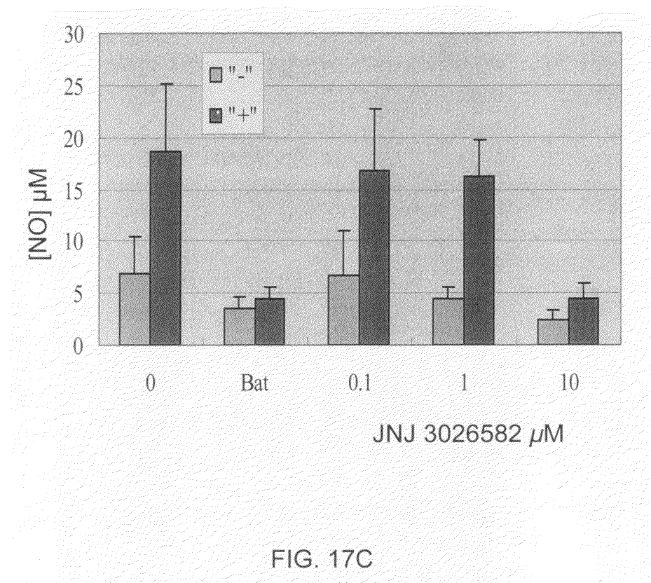

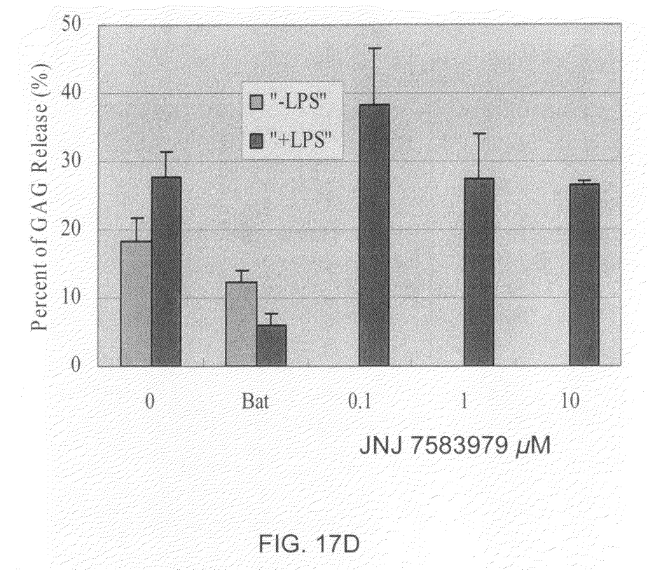

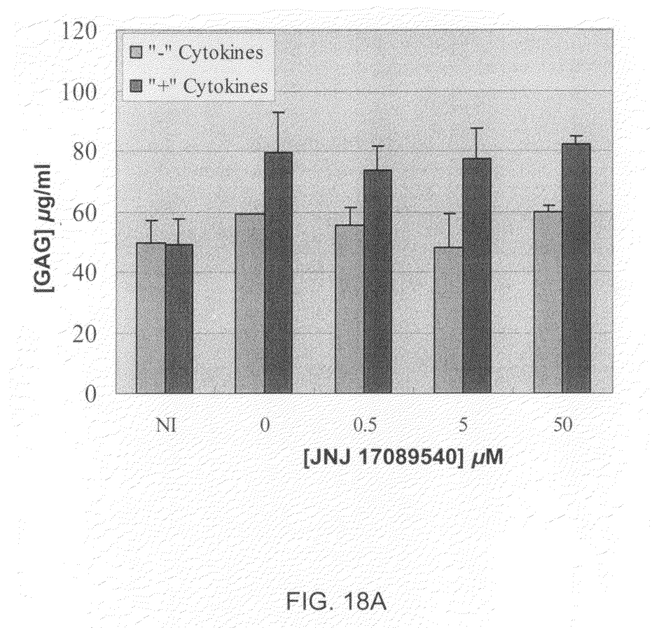

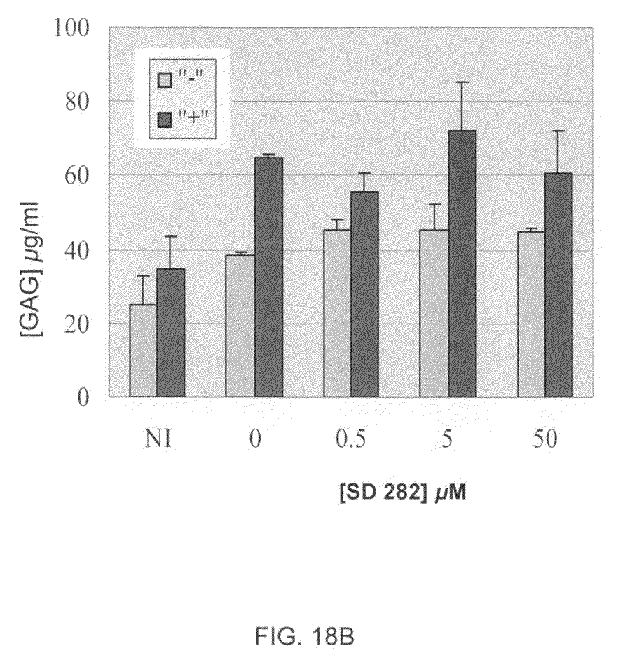

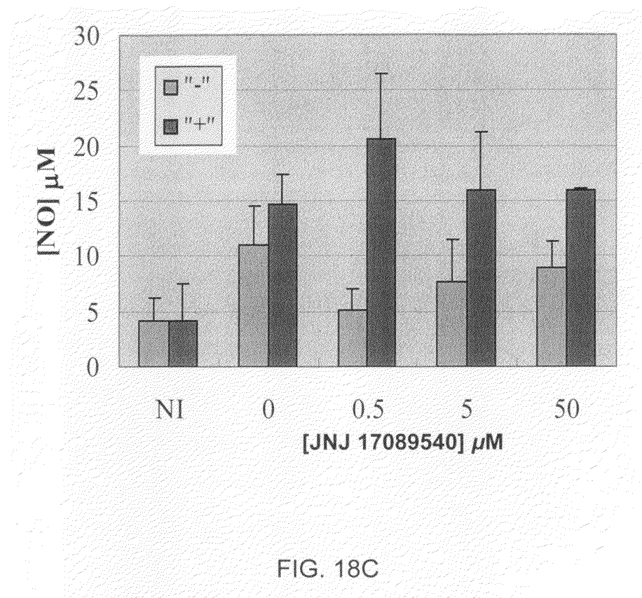

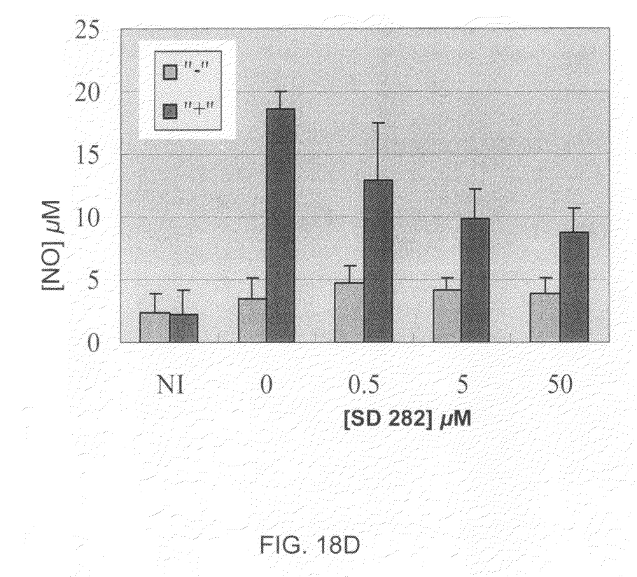

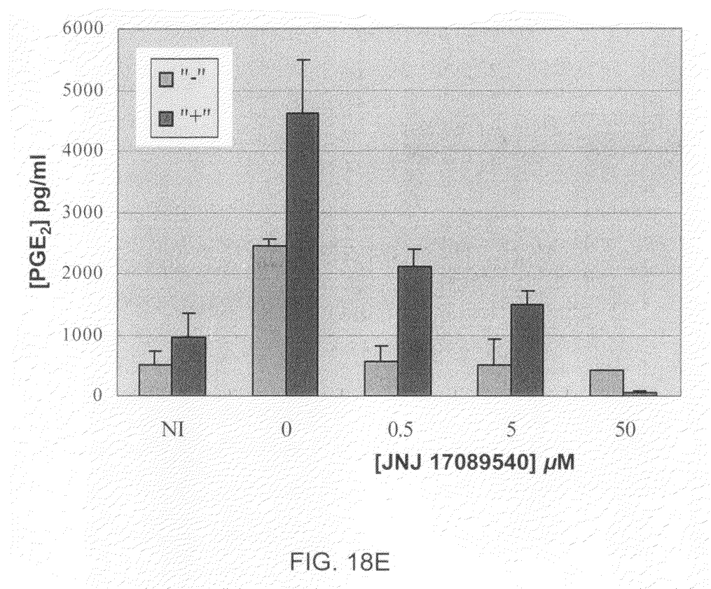

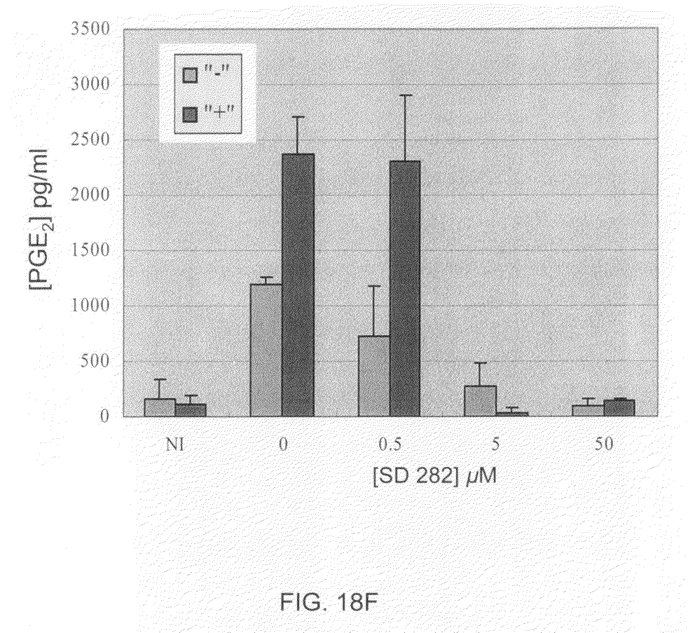

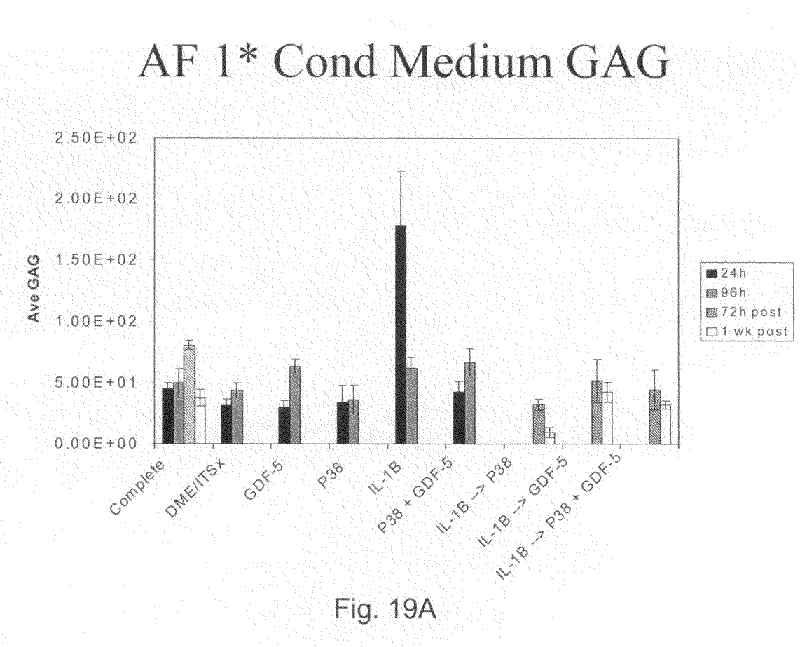

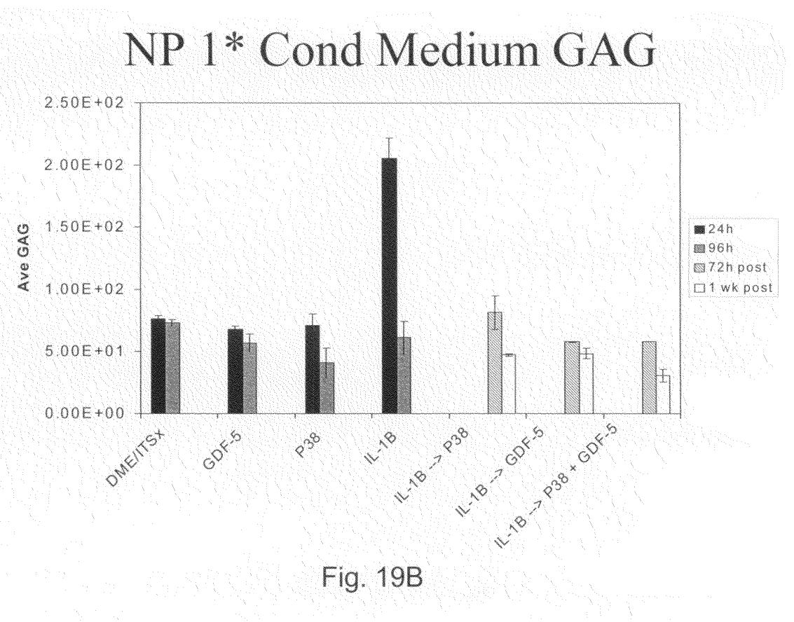

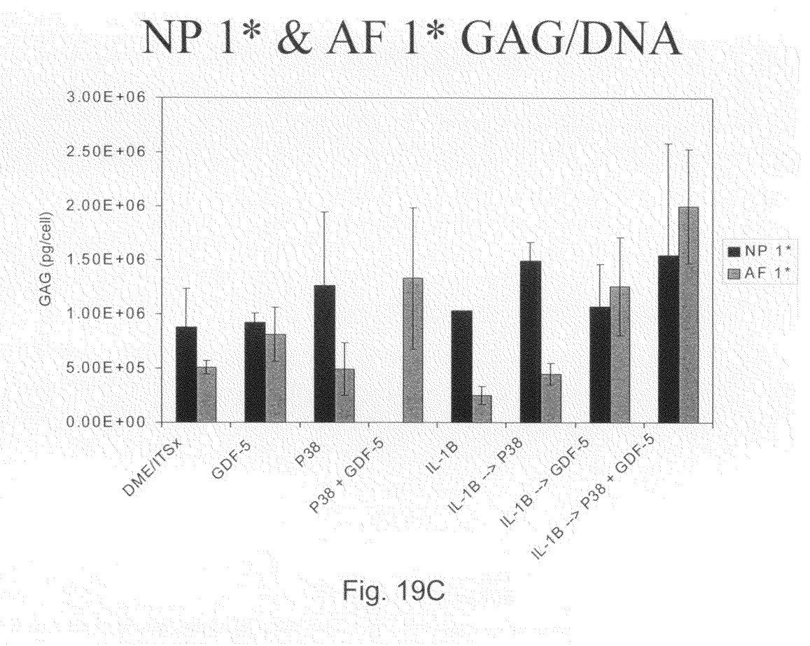

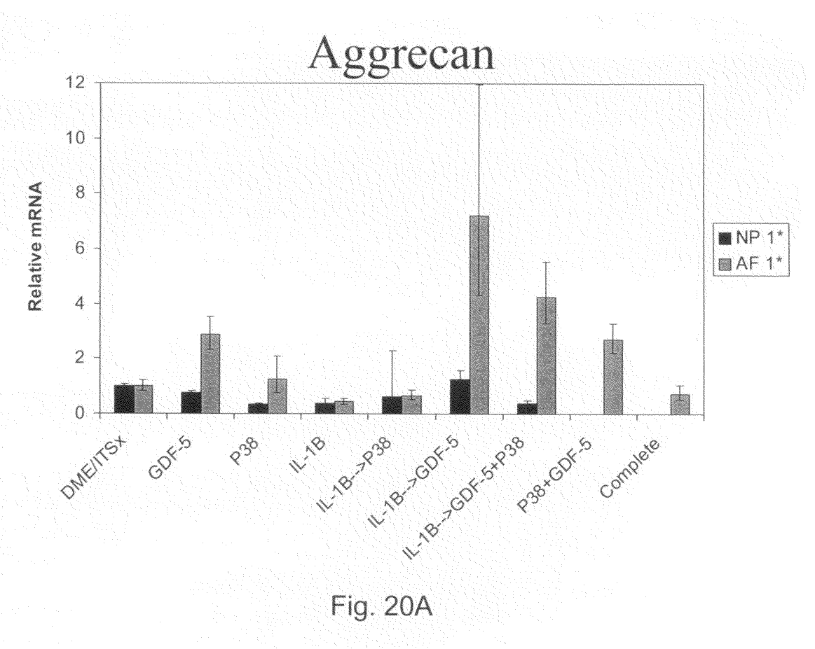

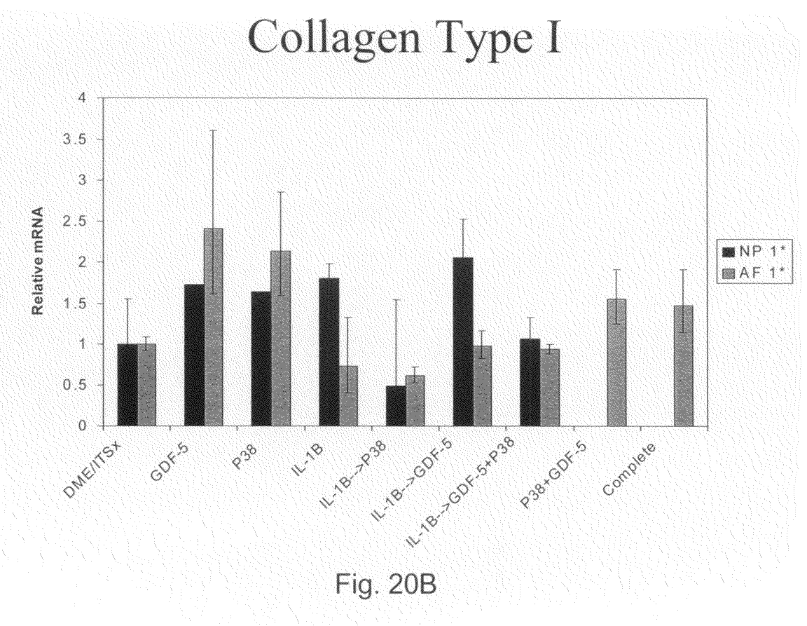

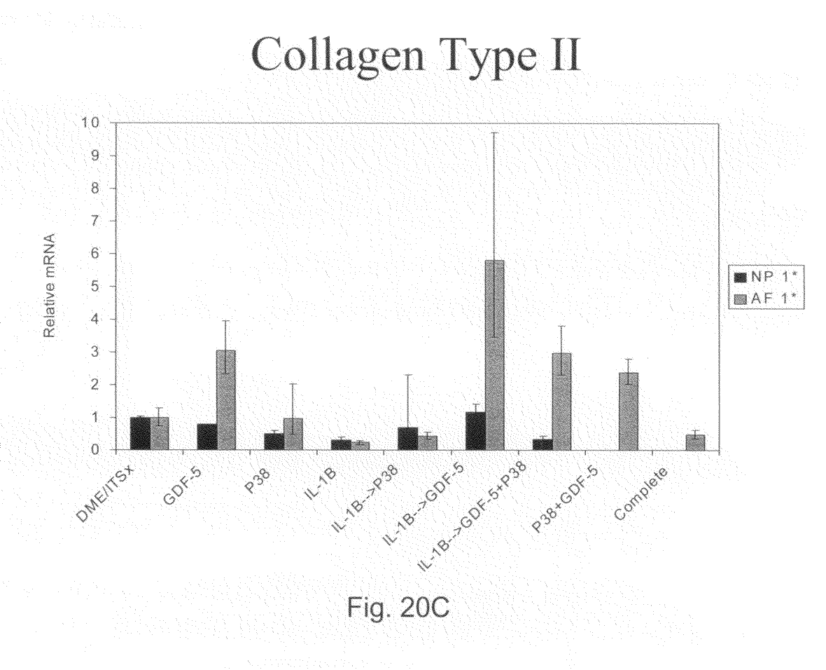

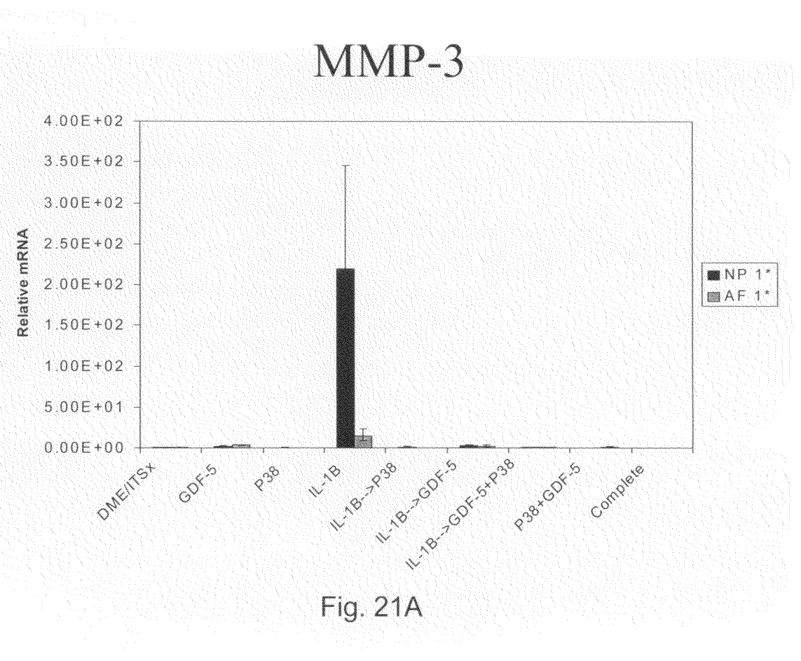

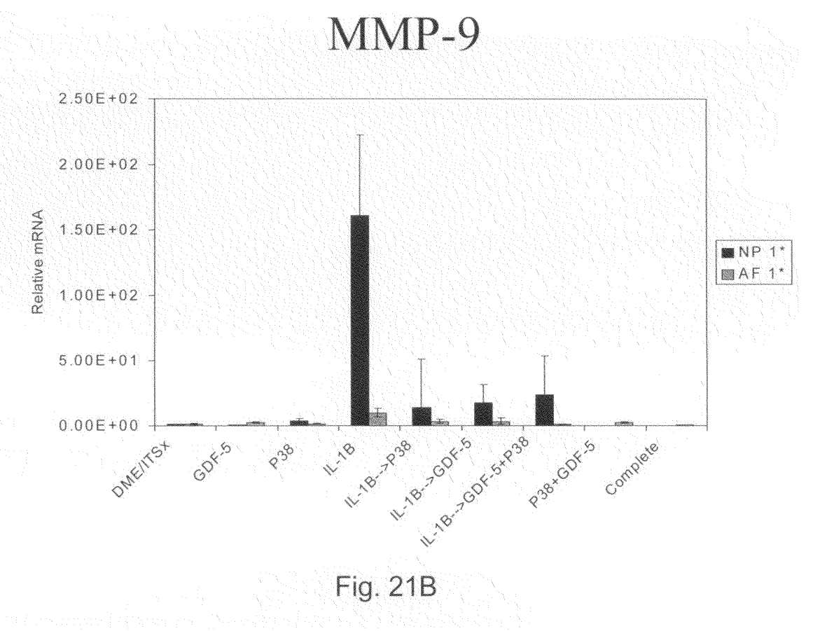

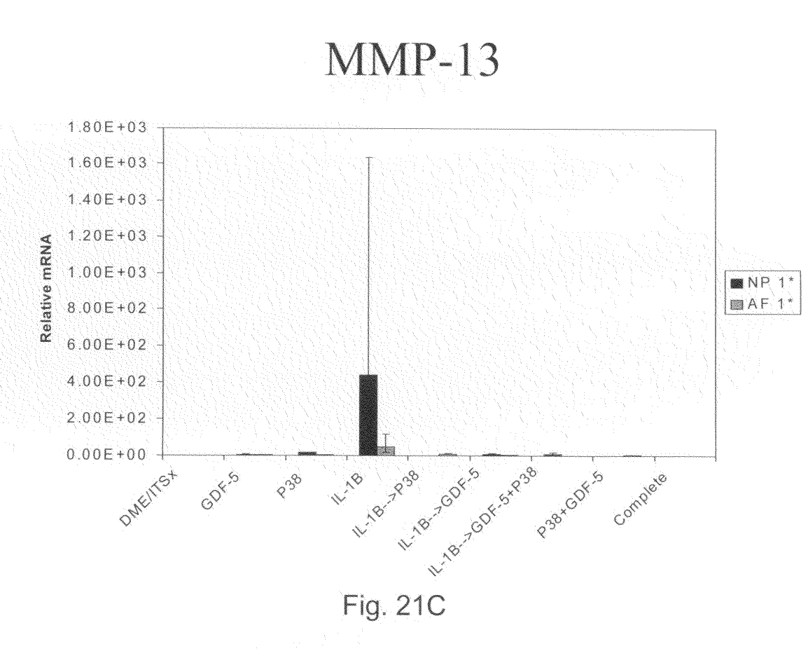

SCIO-469 (SD 469) had no effect on inhibition of GAG degradation in ARC tissues stimulated with IL-1β (FIG. 11A). Cytotoxicity of SCIO-469 (SD 469) was assessed by measuring LDH level in the culture medium and the data indicated that SCIO-469 (SD 469) at 100,000 nM increased LDH level by 121% (FIG. 11B), indicating a mild toxicity at this high concentration to chondrocytes, consistent with the reduced GAG level in the same samples (FIG. 11A). These results suggest that the more water-soluble nature of SCIO-469 (SD 469) may allow better penetration into chondrocyte cells and, hence, that SCIO-469 (SD 469) is more potent than SCIO-282 (SD 282). However, the efficacy in inhibition of PGE₂ by SCIO-469 (SD 469) (maximum inhibition 62%) is lower than SCIO-282 (SD 282) (96%).

To characterize the relative efficacies among different p38 MAP kinase inhibitors, dose-response curves were generated for both PGE₂ and NO assays. Bovine ARCs were treated at 37° C. with serial dilutions of SCIO-469 (SD 469), SCIO-282 (SD 282), JNJ 3026582 (RWJ 67657) and JNJ 17089540 (RWJ 669307) in a range of 1 pM-100 μM. After one hour of treatment, ARCs were stimulated with or without 7.5 ng/ml IL-1β and continued to incubate for five days before assay. The IL-1β-stimulated PGE₂ synthesis or NO production was first calculated by subtraction of PGE₂/NO levels in the absence of IL-1β from that in the presence of IL-1β. Percent of inhibition by compounds was then calculated by the decrease in PGE₂/NO levels in the presence of a compound divided by the IL-1β-stimulated PGE₂/NO levels without a compound treatment. The IC₅₀ values in nM was then calculated using the Sigmoidal dose-response equation: Y=Bottom+(Top-Bottom)/(1+10̂(logIC50−x)) created by Prizm program (Bottom: the lowest effect; Top: the maximum effect). In this plot, the drug concentration point at 0 nM was changed to 0.001 nM, which is ten times lower than the lowest dosage, to avoid the data point at 0 nM being problematic in the program, since Log zero is invalid.

While JNJ 3026582 (RWJ 67657) and SCIO-282 (SD 282) showed similar efficacy in inhibition of both NO and PGE₂, JNJ 17089540 (RWJ 669307) is a weaker inhibitor for NO (FIG. 12A) and even weaker in inhibition of PGE₂ synthesis (FIG. 12B), consistent with its different mechanism of action from those of JNJ 3026582 (RWJ 67657) and SCIO-282 (SD 282).

TABLE 3B
Summary for the compounds tested

| | | Pellet Assay | | |
|---|---|---|---|---|
| Compound | | | PGE2 | |
| category | Compound | GAG Release | Inhibition | NO Inhibition |
| Antiproliferatives | Rapamycin | 50% at 10 μM | 47% at 10 μM | 30% at 10 μM |
| | JNJ 7706621 | 100% at 5 μM | No effect | 100% at 5 μM |
| | (CDK Inhibitor) | | | |
| NSAIDs | Suprofen | No effect | IC50 = 812pM | 20% at 10 μM |
| | Tolmetin | No effect | IC50 = 673 nM | No effect |
| | Tepoxalin | 15% at 10 μM | IC50 = 3.28 μM | 50% 10 μM |
| | Proxicam | No effect | ND | ND |
| | Tiaprofenic | No effect | ND | ND |
| | Acid | | | |
| P38 MAP Kinase | JNJ 3026582 | ~90% inh at 10 μM | IC50 = 421 nM | IC50 = 119 nM |
| Inhibitors | (RWJ 67657) | | | |
| | JNJ 7583979 | 64% at 10 μM, 10% at | 100% at | 50% at 0.5 μM |
| | (RWJ 351958- | 1 μM | 0.5 μM | |
| | 000-A) | | | |
| | JNJ 17089540 | No effect | IC50 = 29.6 μM | IC50 = 4.9 μM |
| | (RWJ669307) | | | |
| | SD-282 | No effect | IC50 = 583 nM | IC50 = 74 nM |
| | SD-469 | No effect | IC50 = 18.01 nM | IC50 = 106.8 |
| Other anti- | Anti-TNF* | 100% at 1 ng/ml | 100% at | 100% at |
| inflammatories | | | 10 ng/ml and | 1 ng/ml |
| | | | 50% at 1 ng/ml | |
| | Topramax | No effect | No effect | No effect |
| | Feverfew | 50% at 0.1 mg/ml and | 62% at | ND |
| | | 22% at 0.05 mg/ml | 0.1 mg/ml and | |
| | | | 23% at | |
| | | | 0.05 mg/ml | |
| | Centella | No effect | 25% at | 25% at |
| | (ETCA) | | 10 μg/ml | 10 μg/ml |
| | Madecassoside | No effect | No effect | No effect |
| Protease | ORC | ND | 100 at | ND |
| Inhibitors | (Interceed)** | | 0.8 mg/ml | |

*The effects of Remicade shown are on TNFα stimulated responses, with no effects on IL-1β stimulated reactions.
**The effects of ORC shown are those in HA solution.

### EXAMPLE IV

### Drop Tower Design Model

In order to assess cartilage breakdown by inflammatory mediators and the effect of antagonists on those mediators, a cartilage impact model (the “drop tower model”) was established using a drop tower device to apply a peak compressive stress to a cartilage sample of about 20-30 MPa over an area of about 11-15 mm². Advantages of this model included its clinical relevance due to its potential to mimic several key parameters of osteoarthritis such as inflammatory cell mediators (by co-culturing with inflammatory cells), and induction of trauma to create the cartilage damage.

Joint impact trauma frequently is sustained through accidents involving falls or direct blows to the joint. Even with careful early surgical reconstruction, development of posttraumatic osteoarthrosis is frequently a late consequence of joint impact trauma, leading to disability and requiring subsequent surgical intervention (such as joint arthroplasty and replacement by prostheses). Although links between a traumatic event and osteoarthrosis have been reported, the cellular pathways underlying progressive cartilage destruction are unknown. Some experimental animal studies reported structural changes in cartilage tissue and changes in cartilage metabolism similar to those found in early osteoarthrosis after impact trauma. Progressive degeneration after initial structural damage may be related to modifications of the mechanical environment or tissue properties. In addition, ongoing structural damage may be associated with biochemical processes after the traumatic event, which augment posttraumatic changes. These biochemical processes likely include inflammatory mediators that contribute to ultimate cartilage degeneration.

In the “drop tower design model” cartilage explants were first injured and then cultured in inflammatory medium to mimic the progression of joint impact trauma linked to cartilage degeneration and osteoarthritis. The approach of the model is to artificially produce cellular damage without gross structural alterations, and to determine how the cells respond to such an insult over the ensuing days when treated with or without potential therapeutic agents.

The drop tower design model was based on previously published papers (Jeffrey, J. E. et al., “Matrix Damage and Chondrocyte Viability Following a Single Impact Load on Articular Cartilage,” Archives of Biochem and Biophys, 322(1): 87-96 (1995); Duda G. N. et al., “Chondrocyte Death Precedes Structural Damage in Blunt Impact Trauma,” Clinc Orth Res, 393: 302-309 (2001); and David M G et al., “Inflammation Enlarges the Zone of Damage of Articular Cartilage After Acute Mechanical Trauma,” American Academy of Orthopaedic Surgeons 393: 302-309 (2002)) with modifications. The contents of these references is incorporated by reference in their entirety.

The device comprises a vertical rail that guides a sliding weight from 0.1 kg to 3 kg directly onto a force transducer which is situated at the bottom of the device. A sphere-shaped 2 mm indenter resting on a specimen is located at the bottom of the vertical rail. The indenter is held by a stop screw. The specimen is rigidly attached to a tissue holder underneath the indenter, to avoid side movements, the specimen is rigidly attached to the tissue holder. In this manner, the force transducer transduces a force indirectly onto the specimen. Adjusting the weight of a mass and height from which the mass drops varies the magnitude of the intended impact. For example, a weight mass (e.g., 500 mg) held by a weight holder can be released from a height of e.g., 36 cm and dropped onto the force transducer.

For this model, the following in vitro parameters were evaluated:
a) histological scoring,
b) Glycosaminoglycan (GAG) release in the media—a measurement of proteoglycan degradation which indicates cartilage extracellular matrix breakdown,
c) GAG content in the tissues by histological stain,
d) PGE₂ levels by Enzyme-linked Immunosorbent Assay (ELISA)—a primary product of arachidonic acid metabolism that is synthesized and released upon cell activation, and whose presence indicates an inflammatory response, and
e) Total Nitric Oxide (NO) production—measurement of NO production indicates the presence of inflammatory response or mitogenic stimuli.

### Pilot Studies:

Pilot studies were performed to determine optimal parameters to generate injury in explants. Blunt trauma to the knee joints of animals in vivo has been shown to induce degenerative changes in the cartilage and alterations in mechanical load are known to modulate matrix biosynthesis in vitro.

To examine if cartilage matrix metabolism is also changed in response to injury in this model, the level of proteoglycan degradation in injured cartilage was determined.

Time course analyses of GAG release in response to various levels of severity of injury on cartilage specimens that were subsequently stimulated with or without PBMC-conditioned medium were performed. The level of GAG released was readily detectable at day three and was higher at day five of incubation. More importantly, the injured cartilage had more GAG degradation than non-injured specimens, which was proportional to the severity of an injury. After seven days in culture, however, cellularity per disc for both injured and non-injured specimens dropped to 14-30% of the cell number found at 1 day of incubation, indicating that there was tissue necrosis with time of culturing. Furthermore, longer incubation resulted in higher basal GAG release that narrowed down the treatment window in this model. Therefore, samples were harvested for assay at day five post treatment. In addition, since there was no gross damage to the cartilage samples even at highest impact energy tested, 1.96J was chosen as the impact energy for all subsequent experiments to ensure a maximal effect on cartilage.

The data demonstrated that cartilage injury resulted in increased GAG degradation (1.5-3 fold increase) and PGE₂ synthesis (1.7 fold) in an inflammatory environment.

### Studies of the Effects of Therapeutic Compounds

### Materials.

Dulbecco's Modified Eagle Medium (D-MEM) with high glucose (Cat. No 10564011), RPMI Medium 1640 (Cat. No. 11835030), Antibiotic-Antimycotic (100×) containing penicillin, streptomycin, amphotericin B, and neomycin (10000 U/ml, 10 μg/ml, 25 mg/ml and 5 μg/ml, respectively) (Cat. No. 15240062), and phosphate buffered saline (PBS, Cat. No. 14130-144) were purchased from Invitrogen Life Technology (Carlsbad, Calif.). Dimethylethylene (341088), ascorbic acid (A-4403), L-proline (P-5607), tolmetin sodium salt dihydrate (T-6779), Diacerein (D9302), Rhein (R-7269), indomethacin (1-7378), lipopolysaccharide (L-6529), insulin-transferrin-sodium selenite media supplement (Cat. No. I-1884), trypan blue (T-8154), and chondroitin 6-sulfate sodium salt from shark cartilage (C-4384) were purchased from Sigma-Aldrich (St. Louis, Mo.). Recombinant human interleukin-1β (IL-1β, Cat. No: 201-LB), recombinant human tissue necrosis factor α (TNF-α) (Cat. No. 210-TA), recombinant interferon-γ (Cat. No. 285-IF), recombinant human IL-6 (Cat. No. 1609-CL-025/CF) and Human IL-6 Quantikine ELISA Kit (Cat. No. D6050) were purchased from R&D Systems (Minneapolis, Minn.). A Nitrate/Nitrite Colorimetric Assay Kit (Cat. No. 780001), and a prostaglandin E2 detection kit (Cat. No. 514131) were purchased from Cayman Chemicals (Ann Arbor, Mich.). A 3 mm biopsy punch (VWR-21909-140) was purchased from VWR International (West Chester, Pa.). Peripheral blood mononuclear cells (PBMC) were purified from fresh bovine or human blood through ficoll gradient centrifugation by Lampire Biologics, Inc (Pipersville, Pa.). A Cytotoxicity Detection Kit (LDH) (Cat. No. 1 644 793) was purchased from Roche (Nutley, N.J.). Fetal calf serum (FCS), Cat. No. SH30070.03, Lot No. ANF19047) was purchased from Hyclone. Papain and a LIVE/DEAD Viability/Cytotoxicity Kit (L-3224) were purchased from Molecular Probes. Collagenase (3.4.24.3) and papain (3.4.22.2) were purchased from Worthington Biochemical Corp. (Lakewood, N.J.). A bone saw was purchased from Mar-med Inc. (Cleveland, Ohio).

### Specimen Preparation and Treatment.

Bovine articular cartilage was obtained from the knee joints of adult, eighteen to twenty-four month old animals and processed within twenty-four hours after sacrifice. After removal of muscle and ligament, tibia plateau and trochlear groove areas were first cut into small pieces in a range of ˜1-3×˜2-5 cm² employing the least curved cartilage surface and ˜1-1.5 cm of the subchondral bone underneath, since the presence of underlying bone has been shown to significantly limit the degree of matrix damage and cell death. Care was taken to consistently prepare the same thickness of bone layer. The cartilage-on-bone specimens were subjected to an impact. Since the contact area between cartilage surface and indenter during impact was about 1-2 mm in diameter, with a minimum of 0.4 cm apart between two adjacent loads, 3-5 impact loads could be performed on a piece of cartilage-on-bone with the above-mentioned dimensions. After each impact, the cartilage area with a visible indent of ˜0.5-1 mm in the center was punched out with a 3 mm diameter puncher and immediately soaked in PBS with 1× Antibiotic-Antimycotic. Throughout the impact procedure and preparation of specimens, the cartilage was kept moist using PBS to prevent tissue dehydration. The cartilage discs were sterilized through 3× one hour wash in PBS with 10× Antibiotic-Antimycotic followed by 1×0.5 hour wash in PBS with 1× Antibiotic-Antimycotic. After wash, the specimens were incubated with conditioned medium harvested from peripheral blood mononuclear cells (PBMC) stimulated with or without LPS and at the same time, were treated with or without compounds for five days at 37° C., 5% CO₂. Culture medium was collected for GAG release, NO and PGE₂ assays, and assessment for cytotoxicity. The cartilage discs were digested with papain for GAG content.

### Assessment of Cell Viability:

Cartilage discs were also processed to assess cell viability using Molecular Probe's Calcein-AM (C-1430) for live cells and ethidium homodimer-1 (E-1169) for dead cells, following previously described methods (Kim, Y J, Sah, R L, Doong J Y, Grodzinsky, A J, “Fluorometric assay of DNA in cartilage explants using Hoechst 33258,” Analytical Biochemistry, 174:168-176 (1988)) with modifications. Briefly, the injured or non-injured explant disc was manually sliced vertically into slices of 0.1-0.5 mm in thickness and incubated in 2 μM calcein AM in PBS for 30-45 min at RT followed by incubation with 4 μM EthD-1 solution for five to ten minutes. The cell viability was assessed under the fluorescence microscope. The absorbance/emission wavelengths are 494/517 nm for Calcein and 528/617 nm for Ethidium homodimer-1 respectively.

To quantitatively assess relative cell death after injury at indicated time periods of incubation with conditioned media from PBMC, cartilage discs were digested with 0.2% collagenase in DMEM supplemented with 10% FCS and 1× Antibiotic-Antimycotic for four hours at 37° C. with gentle agitation. After filtering through a 40 μm Nylon Cell Strainer (Cat. No. REF 352340, BD Falcon) to remove tissue debris, chondrocytes were spun down, washed with 1×PBS and resuspended in 0.2-0.5 ml PBS. Viable and dead cells were counted under a light microscope in the presence of Trypan Blue. The percentage of cell death was calculated by dividing the number of blue stained cells by the total number of cells counted. In a series of digestions of normal bovine cartilage cultured for twenty-four hours, the viability of the cells extracted was found to be 90%. It was assumed that similar conditions prevail in impacted cartilage and that culturing and enzymatic extraction would result in a loss of 10% of the viable cells. The measured viabilities were, therefore, scaled by 100/90 to correct for this, so that the loss of viability shown was that due solely to the impact.

### Induction of Inflammatory Conditions:

Samples for compound evaluation were tested using either of two different methods to induce inflammatory conditions. One method utilized media conditioned from peripheral blood mononuclear cells (PBMCs) stimulated with LPS (“+LPS”) or non-inflammatory media without LPS (“−LPS”). Another method utilized the absence or presence of a cocktail of cytokines.

In the first method, bovine or human PBMCs were freshly prepared using Ficoll gradient by Lampire Biological Laboratories, Inc. PBMCs in RPMI w/10% FCS at delivery were spun down at 1500 rpm for twenty minutes and the cell pellet was washed twice with 1×PBS to wash off the residual Ficoll. The cells were then resuspended at a density of 1×10⁶/ml in RPMI supplemented with 10% FCS, 1× Antibiotics-Antimycotics. The suspension was divided into two aliquots. One aliquot was stimulated with 10 μg/ml of LPS and the other was not stimulated. Both aliquots were then incubated at 37° C., 5% CO₂ for twenty-four to seventy-two hours. The conditioned media with or without LPS stimulation are collected, aliquoted, and stored in −80° C. for a maximum of three months. Each conditioned medium was assessed for levels of pro-inflammatory cytokines to ensure their secretion from PBMC. A representative cytokine, IL-6, was assayed by ELISA using a Human IL-6 Quantikine kit and it was shown that the LPS-stimulated medium had at least 10 ng/ml of IL-6, while IL-6 was undetectable in the unstimulated medium.

In the second method, the explants were cultured at 37° C., 5% CO₂ either in stimulated or non-stimulated conditioned medium as described above or in defined medium containing DMEM supplemented with 1% FCS, 1× insulin-transferrin-sodium selenite media supplement and Antibiotics-Antimycotics in the presence or absence of a cocktail of cytokines containing 10 ng/ml of IL-1β, 100 ng/ml of TNFα and 5 ng/ml of IFN-γ. The compounds were solubilized in H₂O, Ethanol or DMSO (10⁻² M) according to suppliers' instructions and further diluted with either conditioned medium stimulated with or without LPS to the required concentrations. DMSO concentrations in the culture media never exceeded 1%. This concentration of DMSO has no effect on cartilage proteoglycan metabolism in response to cytokines. The injured and noninjured cartilage explants were treated with compound-containing medium for three to five days. The specimens were first treated with compounds at desired concentrations at 37° C. for one hour and then incubated for three to five days in the absence or presence of the cocktail of cytokines. At the end of the incubation, media and cartilage samples were harvested and frozen for further analysis.

### GAG Degradation Assay:

Glycosaminoglycan (GAG) levels in the culture media or cartilage were determined by measuring the amount of polyanionic material reacting with 1,9-dimethylmethylene blue (DME) as detected by absorbance at 525 nm, using shark chondroitin sulfate as a standard. The DME blue dye-binding solution was prepared by dissolving 16 mg of DME blue in a solution containing 0.304% glycine, 0.23% NaCl, 9.5 mM HCl, pH=3.0 with A525 equaling 0.31. Proteoglycans and proteoglycan metabolites in cartilage were released by digestion with 125 μg/ml of papain in 0.1 M PBS, pH 6.0, 5 mM cysteine, 5 mM Na₂EDTA at 50° C. for overnight (Farndale R. W. et al., “Improved Quantitation and Discrimination of Sulphated Glycosaminoglycans by use of Dimethylmethylene Blue,” Biochimica et Biophysica Acta, 883: 173-177 (1986)). Results are reported as either micrograms of GAG per milliliter or percent of total GAG released into culture medium.

### Total Nitrite and Nitrate Assay:

Nitrate/Nitrite was measured using the Griess reaction (Green L. C. et al., “Analysis of Nitrate, Nitrite, and [15N]nitrate in Biological Fluids,” Anal. Biochem. 126: 131-138 (1982)) with a Nitrate/Nitrite Colorimetric Assay Kit (Cayman Chemicals), as described in Example III above.

### Prostaglandin E₂ Assay.

PGE₂ was measured with STAT-Prostaglandin E₂ EIA ELISA kit (Cayman Biochemicals), as described in Example III above. The antibody-PGE₂ complex binds to a goat polyclonal anti-mouse IgG that has been previously attached to the well. After washing to remove any unbound reagents, para-nitrophenyl phosphate (pNPP) was added to the well. The product of this enzymatic reaction absorbs strongly at 412 nm. Because of the large range in response to IL-1β and other cytokines, PGE₂ values were converted to log scale prior to doing t-tests.

### Assessment of Cytotoxicity:

Cell death was assessed by measuring the amount of lactate dehydrogenase (LDH) in the culture supernatant. 10-20 μl of culture medium was incubated with the reaction mixture from the Cytotoxicity Detection Kit (LDH, Roche), as described in Example III above. The amount of formazan formed was measured at 500 nm, which was proportional to the number of dead cells. Since no purified LDH was available for a standard curve at each measurement, an LDH level for 100% dead cells was measured in each experiment. The lysate for the dead cells was prepared as follows: Chondrocytes were enzymatically isolated as described earlier and about 0.1 million cells that were equivalent to the number of cells contained in a 3-mm punch of fresh cartilage explant were lysed in 1 ml of PBS with 1% Triton-100. Approximately 10-20 μl of the lysate was used in the assay as killed cell control.

All assays were done with triplicate or quadruplicate pellets for each dose. Negative and positive controls were included for specimens prepared from each knee.

### Results

Effects of diacerein and rhein on inhibition of GAG release and LDH toxicity

The model was validated with known drugs diacerein and rhein (its active metabolite) as well as batimastat. Injured and non-injured cartilage samples were prepared as described above and incubated at 37° C., 5% CO₂ for five days with stimulated (w/ LPS) or non-stimulated (w/o LPS) PBMC conditioned medium. Both diacerein and rhein, at high dosage, completely inhibited proteoglycan degradation in injured cartilage samples induced by inflammatory mediators. This inhibition was dose dependent (FIGS. 14A and 14B). These data suggest that proteoglycan degradation is a good indicator for a therapeutic effect of agents with different mechanisms of action.

The samples were also assayed for LDH levels in culture medium. Chondrocytes from non-injured cartilage explants were extracted according to the procedures described above and completely lysed with 1% Triton-100 in H₂O. The explants were tolerant to the cytotoxic effects of diacerein and rhein, even at high doses.

### Effects of Batimastat on Inhibition of PGE₂ Synthesis and No Production

Using the drop tower design model, non-injured and injured cartilage explants were prepared as described above and incubated for five days in defined medium with (“+”) or without (“−”) a cocktail of cytokines (10 ng/ml of IL-1β, 100 ng/ml of TNF-α and 5 ng/ml of INF-γ) in the presence or absence of 10 μM of batimastat (British Biotech Pharmaceuticals Ltd.). Culture media were harvested for measurement of PGE₂ and NO as described above. Batimastat did not inhibit PGE₂ synthesis, but did inhibit cytokine-stimulated NO production (FIGS. 15A and 15B). Batimastat was used as a positive control to inhibit GAG degradation since it is a known MMP inhibitor. For consistency, it was used throughout the experiments.

### Effects of Rapamycin on Inhibition of GAG Release and LDH Toxicity

Using the drop tower design model, non-injured and injured cartilage specimens were prepared as described above and subjected to treatment with rapamycin at 0.01, 0.1 and 1 μM dosages for five days at 37° C., 5% CO₂. Both culture medium and cartilage discs were analyzed for GAG content and proteoglycan degradation was expressed as percent of GAG level released into the medium. The same culture medium was assayed for LDH level as above and a supernatant of killed chondrocytes with 10% Triton-X was used as a positive control for the assay.

When compared to the “no treatment” explant, Rapamycin inhibited GAG release completely at as low as 0.1 μM and by ˜84% at 0.0 μM (FIG. 16A). At all testing dosages, rapamycin showed little cytotoxicity (FIG. 16B).

### Effects of p38 Map Kinase Inhibitors on Inhibition of GAG Release, No Production and PGE₂ Synthesis

Injured cartilage specimens were processed and treated with the compounds either in the presence (“+”) or absence (“−”) of cocktail of cytokines (10 ng/ml of IL-1β, 100 ng/ml of TNFα and 5 ng/ml of IFN-γ) (FIGS. 17A, B and C), or in PBMC-conditioned medium stimulated with (“+LPS”) or without (“−LPS”) LPS (FIG. 17D). Culture media were collected for assays. Batimastat was used as a positive control to inhibit GAG degradation because it is an MMP inhibitor.

As shown in FIGS. 17A, B and C, JNJ 3026582 (RWJ 67657) dose-dependently inhibited NO production and PGE₂ synthesis, but had little efficacy in inhibiting GAG release. As shown in FIG. 17D, JNJ 7583979 (RWJ 351958) also had little efficacy in inhibiting GAG release. The efficacies of the tested compounds, as well as additional compounds, on inhibition of GAG degradation, NO production and PGE₂ synthesis are summarized in Table 4.

TABLE 4
Efficacies of Tested Compounds on Inhibition of GAG Degradation,
NO Production and PGE₂ Synthesis

| Compound | GAG | PGE₂ | NO |
|---|---|---|---|
| JNJ 3026582 | little | 33% inh at | 66% at 10 μM |
| (RWJ 67657) | | 1 μM and | |
| | | 90% at 10 μM | |
| JNK 7583979 | little | | |
| (RWJ 351958) | | | |
| Rapamycin | 100% inh at 0.1-1 μM | ND | ND |
| Suprofen | 100% inh at 0.1-1 μM | ND | ND |
| Tolmetin | 60% inh at 10 μM | ND | ND |
| Tepoxalin | 100% inh at 10 μM | 90% inh at | 100% inh at |
| | | 10 μM and | 10 μM |
| | | 77% at 1 μM | |
| Proxicam | No effect | ND | ND |
| Tiaprofenic | 70% inh at 10 μM | ND | ND |
| Acid | | | |
| Rhein | 100% inh at 35 μM | | |
| Diacerein | 100% inh at 54 μM | | |
| Batimastat | 100% inh at 10 μM | No effect | 100% inh at |
| | | | 10 μM |

The effects of JNJ 17089540 (RWJ 669307) and SCIO-282 (SD 282) on inhibition of GAG release, NO production, and PGE₂ synthesis in injured cartilage explants were also assessed. Injured cartilage specimens were processed and treated with the compounds in the presence (“+”) or absence (“−”) of a cocktail of cytokines (10 ng/ml of IL-1β, 5 ng/ml of IFN-γ, 100 ng/ml of TNFα) (FIGS. 18A-F). Culture media were collected for assays as described above. A non-injured cartilage specimen was included as a negative control.

While the p38 MAP kinase inhibitors showed little efficacy in inhibition of GAG release (FIGS. 17A, 17D and 18A-B), they dose-dependently inhibited both NO production (FIGS. 17C and 18C-D) and PGE₂ synthesis (FIGS. 17B and 18E-F), indicating that this class of compounds mainly modulates MAP kinase activity that might contribute to aberrant function of downstream transcription factors involving in NO and PGE₂ synthesis.

The results from the chondrocyte pellet model and the drop tower design model may demonstrate different effects of the compounds tested because, while both models propagate osteoarthritis, the chondrocyte pellet culture model is more sensitive to treatment, including treatment by soluble factors and more control of variability between samples, and the drop tower model is a better mimic of in vivo conditions than the chondrocyte pellet culture model, having a cellularity (0.2 M vs. 2 M for pellets) and percent of collagen more similar to actual tissue. For this reason, both models were used to obtain data.

### EXAMPLE V

### 3D In Vitro Model for Testing Drug Potency on Cytokine-Mediated Inflammation in Disc Cells

Three-dimensional in vitro annulus fibrosis (AF) and nucleus pulposus (NP) models were established to investigate proteoglycan modulation and changes in gene expression in response to inflammatory cytokines such as interleukin-1 (e.g., IL-1β) and potential therapeutic agents such as growth and differentiation factor (GDF-5), and p38 MAP kinase inhibitors.

AF and NP cells from bovine caudal discs were isolated by enzymatic digestion and seeded (2×10⁶ cells/cm²) onto Millipore CM filters coated with Collagen Type II and cultured in DMEM/20% heat-inactivated FBS, 100 ug/ml ascorbic acid and 5 ng/ml TGFβ1. After approximately three weeks in culture, medium was changed to DMEM/ITSx (insulin, transferring, selenium) and 10 ng/ml IL-1β was added to the cultures (4 days) to measure the amount of proteoglycan release in conditioned medium and in the tissue. Finally, rhGDF-5 (200 ng/ml) alone, and p38 MAP kinase inhibitor JNJ3026582 (RWJ 67657) at 1 μM/ml alone, and the combination of the two, were added to the cells to reverse loss of proteoglycans and conditioned media was harvested.

Results were evaluated by histology (Tol. Blue staining was used to observe matrix production). Dimethylmethylene blue (DMMB) was used to detect sulphated glycosaminoglycans in a GAG/DNA assay. Taqman analysis was performed on aggrecans (AGG), collagen type 1 (col-1), collagen type 2 (col-2), and MMP 3, MMP 9 and MMP 13.

The results indicated that the p38 MAP kinase inhibitor JNJ3026582 (RWJ 67657) and/or rhGDF-5 restored the amount of GAG in the tissue and gene expression of Agg and Col 2 to close to control levels.

Preliminary data has shown that when the tissue was cultured with IL-1 alone GAG levels dropped to less than 50% as compared to the controls. The p38 kinase inhibitor+/−rhGDF-5 appeared to prevent GAG release in a dose-dependent manner. After adding p38 MAP kinase inhibitor and rhGDF-5 to the IL-1 group, the amount of GAG in the tissue recovered close to the control levels. Initial assessment of P38 MAP kinase inhibitor potency and rhGDF-5 suggests that it may be used to modulate cytokine-induced GAG release. FIGS. 19A-C, 20A-C and 21A-C show the results. These results indicate that the P38 MAP kinase inhibitor and rhGDF-5 enhance the synthesis of extracellular matrix and inhibits its degradation.

In FIGS. 19A-C and 20A-C, annulus fibrosus and nucleus pulposus cells from bovine caudal discs were isolated by enzymatic digestion and seeded (2×10⁶ cells/cm₂) onto Millipore CM filters coated with Collagen Type II and cultured in DMEM/20% heat-inactivated FBS/100 ug/ml ascorbic acid and 5 ng/ml TGF-β1. After three weeks in culture, the medium was changed to DMEM/ITSx (insulin, transferring, selenium) and 10 ng/ml IL-1β was added to the cultures (4 days). rhGDF-5 (200 ng/ml) and-/or p38 MAP kinase inhibitor at 1 μM was added to the cells.

This data further validates the utility of the inventors' use of the in vitro cellular system to model IL-1 induced degeneration (i.e., loss of proteoglycan) and the effects of potential therapeutic agents to reverse the effects of this inflammatory cytokine.

While this invention has been particularly shown and described with references to preferred embodiments thereof, it will be understood by those skilled in the art that various changes in form and details may be made therein without departing from the scope of the invention encompassed by the appended claims.

All references cited herein are incorporated by reference in their entirety.

1. A method of treating degenerative disc disease in an intervertebral disc having a nucleus pulposus and an annulus fibrosus, comprising transdiscally administering an effective amount of a formulation comprising a p38 MAP kinase inhibitor, wherein the p38 MAP kinase inhibitor is selected from the group consisting of:
i) an aryl-pyridinyl heterocycle and
ii) an indol-5-carboxamide.

2. The method of claim 1 wherein the p38 MAP kinase inhibitor is selected from the group consisting of:
i) diaryl imadazole;
ii) N,N′-diaryl urea;
iii) N,N-diaryl urea;
iv) benzophenone;
v) pyrazole ketone;
vi) indole amide;
vii) diamides;
viii) quinazoline;
ix) pyrimido [4,5-d]pyrimidinone;
x) pyridylamino-quinazolines;
xi) JNJ 3026582 (RWJ 67657);
xii) JNJ 17089540 (RWJ 669307);
xiii) JNJ 7583979 (RWJ 351958);
xiv) SCIO-282 (SD 282); and
xv) SCIO-469 (SD 469).

3. A method of inhibiting TNFα-induced glycosaminoglycan release in an intervertebral disc having a nucleus pulposus and an annulus fibrosus, comprising transdiscally administering an effective amount of a formulation comprising a p38 MAP kinase inhibitor and a TNFα antagonist into an intervertebral disc, wherein the p38 MAP kinase inhibitor is selected from the group consisting of:
i) JNJ 3026582 (RWJ 67657);
ii) JNJ 17089540 (RWJ 669307);
iii) JNJ 7583979 (RWJ 351958);
iv) SCIO-282 (SD 282); and
v) SCIO-469 (SD 469).

4. The method of claim 1, wherein the p38 MAP kinase inhibitor is a aryl-pyridinyl heterocycle.

5. The method of claim 1, wherein said p38 MAP kinase inhibitor is an indol-5-carboxamide.

6. The method of claim 1, wherein the formulation is administered in an amount effective to reduce pain.

7. The method of claim 1, wherein the formulation is administered in an amount effective to inhibit degradation of an extracellular matrix of the nucleus pulposus.

8. The method of claim 1, wherein the formulation is provided closely adjacent the outer wall of the annulus fibrosus.

9. The method of claim 1, wherein a portion of the nucleus pulposus is removed prior to transdiscally administering the formulation.

10. The method of claim 1, wherein the administration comprises providing the formulation in a depot at a location closely adjacent to an endplate of an adjacent vertebral body.

11. The method of claim 1, wherein the formulation further comprises at least one additional therapeutic agent.

12. The method of claim 11, wherein the additional therapeutic agent is selected from the group consisting of:
i) a growth factor,
ii) mesenchymal stem cells, adult stem cells and embryonic stem cells,
iii) an MMP antagonist,
iv) a monoclonal anti-TNFα antibody,
v) rapamycin,
vi) glycosaminoglycans.
vii) an antagonist of nitric oxide synthesis,
viii) an anti-oxidant,
ix) an anti-proliferative agent,
x) an anti-apoptotic agent,
xi) a COX-2 antagonist,
xii) a non-steroidal anti-inflammatory agent,
xiii) batimistat,
xiv) rhein,
xv) diacerein,
xvi) an IL-6 antibody, and
xvii) rhGDF-5.
xviii) glycosaminoglycans,
xvix) microparticles,
xx) a caspase inhibitor,
xxi) an inhibitor of pro-inflammatory interleukin,
xxii) an inhibitor of PLA₂ enzyme,
xxiii) tetracycline analogs and
xiv) IGFI and II.

13. The method of claim 11, wherein the additional therapeutic agent comprises mesenchymal stem cells.

14. The method of claim 11, wherein the additional therapeutic agent is selected from the group consisting of: rapamycin, JNJ 7706621, tolmetin, tepoxalin, suprofen, tiaprofenic acid; centella (ETCA), madecassoside, rhein, diacerein, feverfew, batimastat and ORC (Interceed).

15. The method of claim 1, wherein the formulation is predominantly released from the formulation through a sustained delivery device.

16. A method of preventing or therapeutically treating a degenerating intervertebral disc, comprising the steps of:
a) determining a level of a pro-inflammatory protein within the disc,
b) comparing the level against a pre-determined level of the pro-inflammatory protein, and
c) injecting a p38 MAP kinase inhibitor into the disc, wherein the p38 MAP kinase inhibitor is selected from the group consisting of:
i) JNJ 3026582 (RWJ 67657);
ii) JNJ 17089540 (RWJ 669307);
iii) JNJ 7583979 (RWJ 351958);
iv) SCIO-282 (SD 282); and
v) SCIO-469 (SD 469).

17. The method of claim 16, wherein the pro-inflammatory protein is a p38 MAP kinase, interleukin-1, interleukin-4, interleukin-6, interleukin-8, or TNF-α.

18-37. (canceled)
